(19) United States
(12) Patent Application Publication
(10) Pub. No.: US 2022/0031654 A1
(43) Pub. Date: Feb. 3, 2022

(54) Standardized Bioflavonoid Composition for Regulation of Homeostasis of Host Defense Mechanism

(71) Applicant: Unigen, Inc., Tacoma, WA (US)

(72) Inventors: Mesfin Yimam, Tacoma (US); Teresa Horm, Renton (US); Qi Jia, Olympia (US); Lidia Brownell, Tacoma, WA (US); Alexandria O'Neal, Tacoma, WA (US)

(73) Assignee: Unigen, Inc., Tacoma, WA (US)

(21) Appl. No.: 17/389,001

(22) Filed: Jul. 29, 2021

Related U.S. Application Data
(60) Provisional application No. 63/058,698, filed on Jul. 30, 2020.

(51) Int. Cl. A61K 31/353; A61K 45/06; A61P 29/00; A61P 11/00

(52) U.S. Cl. CPC A61K 31/353; A61P 11/00; A61P 29/00; A61K 45/06

(57) ABSTRACT

Bioflavonoid compositions for establishment and regulation of homeostasis of host defense mechanism, are disclosed and comprise at least one standardized bioflavonoid extract enriched for at least one free-B-ring flavonoid and at least one standardized bioflavonoid extract enriched for at least one flavan. Contemplated compositions are effective for respiratory diseases and conditions.

This United States patent application claims priority to U.S. Provisional Patent Application Ser. No. 63/058,698 filed on Jul. 30, 2020 and entitled “Standardized Bioflavonoid Compositions for Regulation of Homeostasis of Host Defense Mechanism”, which is commonly-owned and incorporated herein in its entirety by reference.

### BACKGROUND

Aging, a natural phenomenon, is a complicated degenerative process that affects both bodily and mental function over time, and poor host defense response is one of the most observed changes in the senile. Understanding the underlying mechanisms in the decline of the host defense response that occurs in the elderly is a key first step in its mitigation. Chemically induced accelerated aging models, such as the D-Galactose-induced thymus damage and immune senescence mouse model, is one of the preferred options to study the impacts of aging on the immune system. In the chemically induced animal aging models, animals exhibit immune senescence that mimics a decline in host defense response frequently observed in the elderly (Azman 2019). D-Galactose induced aging model is one of the commonly used and well-validated animal models in anti-aging research. While it is converted to glucose at normal concentrations in the body, high concentrations of D-Galactose could easily be converted to aldose and hydroperoxide, leading to production of oxygen derived free radicals. It could also react with free amines of proteins and peptides to produce advanced glycation end products (AGEs) through non-enzymatic glycations. Accumulation of these reactive oxygen species (ROS) and increased AGEs in this model would result in disequilibrium of normal organ and host defense homeostasis, which subsequently could cause oxidative stress, systemic inflammation, decreased immune response, mitochondrial dysfunction, and apoptosis (e.g. of thymus cells) that ultimately accelerates the aging process. These changes are among the naturally occurring pathological characteristics of senescence and aging.

Sepsis represents life-threatening organ dysfunction caused by a dysregulated host defensive response to an infection with a potential of organ failure. It is a state mediated principally by macrophages/monocytes attributed to excessive production of several early phase cytokines such as TNF-α, IL-1, IL-6 and gamma interferon as well as late stage mediators such as HMGB1. High-mobility group box protein 1 (HMGB1) is a nuclear or cytosolic endogenous damage-associated molecular pattern (DAMP) protein that can be released or secreted from cells due to damaging stimuli or cytokines. While nuclear HMGB1 is an architectural chromatin-binding factor responsible for maintaining genome integrity, extracellular HMGB1 released from activated or damaged cells is a mediator of inflammation and immune dysfunction in response to various stresses, such as oxidative damage, and pathogen infection. HMGB1, is a critical mediator of sepsis as it is released from activated macrophages and monocytes in response to endogenous and exogenous inflammatory signals (Wang et al., 1999) which could escalate the off balance of host defense mechanism and lead to multiple organ failure and ultimately death. Surviving patients could have an ongoing inflammatory response that may well be driven by the late and continued release of HMGB1 (Gentile and Moldawer, 2014).

Once released actively from stimulated mononuclear cells and passively from necrotic cells, HMGB1 acts as an alarmin (danger signal) addressing a loss of intracellular homeostatic balance to neighboring cells serving to activate the host immune response. It plays a critical role in activation of the innate immune response, by functioning as a chemokine facilitating movement of immune cells to sites of infection, and as a DAMP, activating other immune cells to secrete pro-inflammatory cytokines (Yang et al., 2001). When pro-inflammatory cytokines are produced at low (optimum) levels, they will yield a protective function against viral or microbial invasion; however, if they are overproduced as in the case of a ‘cytokine storm’, they may become harmful to the host by mediating an injurious inflammatory response. In most cases, for hosts with underlying conditions, such as immunodeficiency or compromised immunity and in the elderly, these inflammatory cytokine storms seem to cause acute systemic inflammatory syndrome; If the patient survives, delayed mediation of inflammation may follow, which could result in persistent inflammatory, immunosuppressive and catabolic responses. Besides serving as a chemoattractant for a number of cell types, including all inflammatory cells, HMGB1 causes inflammatory cells to secrete TNF-α, IL-1β, IL-6, IL-8, and macrophage inflammatory protein (MIP) suggesting its participation in a ‘cytokine storm’ (Bianchi and Manfredi, 2007) through activation of NFκB signaling. Significant studies have also reported extracellular HMGB1 can trigger a devastating inflammatory response which promotes the progression of sepsis and acute lung injury (Entezari et al., 2014). In contrast to TNF-α and IL-1β, which are secreted within minutes of endotoxin stimulation, HMGB1 is secreted after several hours, both in vitro and in vivo, indicating its late stage inflammatory mediation. In fact, when HMGB1 neutralizing antibodies were administered 24 hours after the onset of sepsis, they provided protection against lethal endotoxemia, indicating the key role of HMGB1 as a late mediator of lethal sepsis (Wang et al., 1999). Clinically, a strong association had also been established between persistently high level of HMGB1 and subjects in the late stage of sepsis or who succumbed from sepsis (Angus et al., 2007). Recently, some clinical studies have shown that chloroquine and its analogues (hydroxychloroquine) are beneficial for the clinical efficacy and viral clearance of COVID-19 (Andersson et al. 2020, Gao et al., 2020; Gautret et al., 2020). Tested in mouse sepsis model, chloroquine, the anti-malaria drug, prevented lethality where the protective effects were mediated through inhibition of HMGB1 release from macrophages, monocytes, and endothelial cells, thereby preventing HMGB1 cytokine-like activities and inhibition of NF-κB activation (Yang et al., 2013). Dietary antioxidants have been reported with significant attenuation of hyperoxia-induced acute Inflammatory lung injury by enhancing macrophage function via reducing the accumulation of airway HMGB1 (Patel et al, 2020). Hence, the natural bioflavonoid composition containing Free-B-Ring flavonoids and flavans described in the body of the current subject matter with a confirmed inhibition of HMGB1 and NF-κB, prevention of sepsis lethality, inhibition of AGE formation, induction of endogenous antioxidant enzyme, promotion of macrophages phagocytosis, increase bacterial clearance, protection of acute lung injury and safe historical usage to be applied for maintain and protection of respiratory and lung health, prevention and treatment of pathological conditions such as lung injury caused by viral, microbial infections (e.g. COVID-19) and PM2.5 air pollutants, PM10 particles in air, air pollutants, oxidative smog, smoke from tobacco, electronic cigarette, smoke of recreational marihuana.

Acacia catechu Willd (Farbaceae), commonly known as cutch tree, Khair, Khadira, is used as traditional herbal medicine in India and other regions of Asia (Hazral et al., 2017). It is a medium sized (up to 15 m) deciduous tree. The bark is dark grayish brown, exfoliating in long, narrow strips; leave pinnate, with a pair of prickles at the base of the rachis, flowers pale-yellow in cylindrical spike; pods glabrous, flat, and oblong. The Ayurvedic Pharmacopoeia of India describes the heartwood of Acacia catechu as light-red, turning brownish-red to nearly dark with age; attached with whitish sapwood; fracture hard; tasteless, astringent. The moderate size trees, about 8 years or older, are harvested for the extraction of Acacia catechu extract. Plant material supply and plant authentication is the main focus of the initial vendor qualification as the physical appearance of Acacia catechu (a timber), Uncaria gambir (a vine) and cashew nut testa (nut skin) are very different. Acacia catechu has been used in ayurvedic medicine in throat, mouth and gums, also in cough and diarrhea. Externally it is employed as an astringent and as a cooling application to ulcers, boils and receptions on the skin. Powder is used in wound healing treatment. Acacia catechu has been found to increase the number of antibody-producing cells in the animal spleen, indicative of a heightened immune system, increased phagocytosis of macrophages, and inhibit the release of pro-inflammatory cytokines (Sunil et al, 2019).

Scutellaria baicalensis Georgi (Lamiaceae), common name Chinese Skullcap (Huang Qin), is a traditional herbal medicine used in several countries in Asia as indicated in the Chinese Pharmacopeia. The plant is a bushy perennial with reclining to upright stems tinged with purple. Leaves are borne on short stalks and have lance-shaped, hairy, medium green leaves. Racemes of hairy flower with dark blue uppers lips and paler blue beneath bloom from early summer to early fall. During the spring or summer, the two-year-old roots are collected, and air dried for commercial purpose. Based on the Chinese Pharmacopeia, the roots appear as 8˜25 cm long, 1˜3 cm in diameter. It is brownish-yellow or dark yellow externally bearing sparse traces of rootles. The upper part is rough with twisted longitudinal wrinkles or irregular reticula, the lower part with longitudinal striations and fine wrinkles. Texture is hard and fragile, easily broken, facture yellow, reddish-brown in the center; the central part of an old root dark brown or brownish-black, withered or hollowed. It has slight odor and tastes bitter. The dry roots normally contain less than 10% bioflavonoids such as baicalin. The roots used for the Scutellaria extract are examined based on the identification and quantification methods of the Chinese Pharmacopeia by TLC and HPLC methods.

Scutellaria baicalensis was recorded in a classical Chinese medical literature <Shen Nong Ben Cao> from the Eastern Han dynasty (circa 200 C.E. or 2200 years ago). A recent list of the top 30 herbs in Traditional Chinese Medicine (TCM) for treating respiratory infections based on the analysis of two TCM Databases (World Traditional Medicine Patent Database (WTM) and Saphron TCM database) put Radix Scutellaria at the second most utilized herb, with a 38% frequency in all TCM compositions for treatment of respiratory infections (Ge et al. 2010).

Radix Scutellaria was included in TCM compositions recommended by the Chinese government in 2003 during the SARS epidemic. The use of Baicalin (Yuan et al, 2009) and flavonoids from Scutellaria plants (Zhong, et al., 2006) later were patented for SARS and COVID-19 treatment (Song et sl. 2020). Modern scientific studies of Radix Scutellaria identified bioflavonoids especially Baicalin and Baicalein, as bioactive components of this herb (Béjar et al., 2004) with biological functions related to antioxidation, anti-inflammation, reduction of the allergic response, and antibacterial activity (Shen et al, 2021). Baicalin and Baicalein also exhibited potent antiviral activity through the inhibition of proteins that viruses need to bind to and bud from host cells, activities which are essential for infection (Yu et al, 2011). In mice infected with Influenza A H1N1 virus (swine flu), extract from Radix Scutellaria modulated their inflammatory response to reduce disease severity, decreased lung tissue damage, and ultimately increased their survival rate (Zhi et al, 2019).

Flavonoids are a widely distributed group of natural products. The intake of flavonoids has been demonstrated to be inversely related to the risk of incident dementia. The mechanism of action, while not known, has been speculated as being due to the anti-oxidative effects of flavonoids (Commenges et al. 2000). Polyphenol flavones induce programmed cell death, differentiation and growth inhibition in transformed colonocytes by acting at the mRNA level on genes including cox-2, Nuclear Factor kappa B (NFκB) and bcl-X(L) (Wenzel et al. 2000). It has been reported that the number of hydroxyl groups on the B ring is important in the suppression of cox-2 transcriptional activity (Mutoh et al. 2000).

Free-B-Ring flavonoids are relatively rare. Out of a total 9,396 flavonoids synthesized or isolated from natural sources, only 231 Free-B-Ring flavonoids are known. (The Combined Chemical Dictionary, Chapman and Hall/CRC, Version 5:1 Jun. 2001). Free-B-Ring flavonoids have been reported to have diverse biological activity. For example, galangin (3,5,7-trihydroxyflavone) acts as an antioxidant and free radical scavenger and is believed to be a promising candidate for anti-genotoxicity and cancer chemoprevention (Heo et al. 2001). It is an inhibitor of tyrosinase monophenolase (Kubo et al. 2000), an inhibitor of rabbit heart carbonyl reductase (Imamura et al. 2000), has antimicrobial activity (Afolayan and Meyer 1997) and antiviral activity (Meyer et al. 1997). Baicalein and two other Free-B-Ring flavonoids, have antiproliferative activity against human breast cancer cells (So et al. 1997).

Typically, flavonoids have been tested for activity randomly based upon their availability. Occasionally, the requirement of substitution on the B-ring has been emphasized for specific biological activity, such as the B-ring substitution required for high affinity binding to p-glycoprotein (Boumendj el et al. 2001); cardiotonic effect (Itoigawa et al. 1999), protective effect on endothelial cells against linoleic acid hydroperoxide-induced toxicity (Kaneko and Baba 1999), COX-1 inhibitory activity (Wang, 2000) and prostaglandin endoperoxide synthase (Kalkbrenner et al. 1992). Only a few publications have mentioned the significance of the unsubstituted B-Ring of the Free-B-Ring flavonoids. One example is the use of 2-phenyl flavones, which inhibit NADPH quinone acceptor oxidoreductase, as potential anticoagulants (Chen et al. 2001).

The reported mechanism of action related to the anti-inflammatory activity of various Free-B-Ring flavonoids has been controversial. The main bioactive Free-B-Ring flavonoids of Scutellaria baicalensis were reported alleviation of inflammatory cytokines (Liao, et al, 2021). The anti-inflammatory activity of the Free-B-Ring flavonoids, chrysin (Liang et al. 2001), wogonin (Chi et al. 2001) and halangin (Raso et al. 2001) have been associated with the suppression of inducible cyclooxygenase and nitric oxide synthase via activation of peroxisome-proliferator activated receptor gamma (PPARγ) and influence on degranulation and AA release (Tordera et al. 1994). It has been reported that oroxylin, baicalein and wogonin inhibit 12-lipoxygenase activity without affecting cyclooxygenases (You et al. 1999). More recently, the anti-inflammatory activity of wogonin, baicalin and baicalein has been reported as occurring through inhibition of inducible nitric oxide synthase and cox-2 gene expression induced by nitric oxide inhibitors and lipopolysaccharide (Chen et al. 2001). It has also been reported that oroxylin acts via suppression of NFκB activation (Chen et al. 2001). Finally, wogonin reportedly inhibits inducible PGE2 production in macrophages (Wakabayashi and Yasui 2000).

Catechin is one of the well-documented bioactive flavonoids (Bae et al. 2020). Catechin and its isomer epicatechin inhibit prostaglandin endoperoxide synthase with an IC₅₀ value of 40 μmon (Kalkbrenner et al. 1992). Five flavan-3-ol derivatives, including (+)-catechin and gallocatechin, isolated from four plant species: Atuna racemosa, Syzygium carynocarpum, Syzygium malaccense and Vantanea peruviana, exhibit equal to or weaker inhibitory activity against COX-2, relative to COX-1, with IC₅₀ values ranging from 3.3 μM to 138 μM (Noreen et al. 1998). (+)-Catechin, isolated from the bark of Ceiba pentandra, inhibits COX-1 with an IC₅₀ value of 80 μM (Noreen et al. 1998). Commercially available pure (+)-catechin inhibits COX-1 with an IC₅₀ value of around 183 to 279 μM, depending upon the experimental conditions, with no selectivity for COX-2. (Noreen et al. 1998).

To date, approximately 330 compounds have been isolated from various Acacia species. Flavans, a type of water-soluble plant pigments, are the major class of compounds isolated from Acacias. Approximately 180 different flavonoids have been identified, 111 of which are flavans. Terpenoids are second largest class of compounds isolated from species of the Acacia genus, with 48 compounds having been identified. Other classes of compounds isolated from Acacia include, alkaloids (28), amino acids/peptides (20), tannins (16), carbohydrates (15), oxygen heterocycles (15) and aliphatic compounds (10). (Buckingham, The Combined Chemical Dictionary, Chapman and Hall CRC, version 5:2, December 2001).

Green tea catechin, when supplemented into the diets of Sprague Dawley male rats, lowered the activity level of platelet phospholipase A₂ and significantly reduced platelet cyclooxygenase levels (Yang et al. 1999). Catechin and epicatechin reportedly weakly suppress cox-2 gene transcription in human colon cancer DLD-1 cells (IC₅₀=415.3 μM) (Mutoh et al. 2000). The neuroprotective ability of (+)-catechin from red wine results from the antioxidant properties of catechin, rather than inhibitory effects on intracellular enzymes, such as cyclooxygenase, lipoxygenase, or nitric oxide synthase (Bastianetto et al. 2000). Catechin derivatives purified from green tea and black tea, such as epigallocatechin-3-gallate (EGCG), epigallocatechin (EGC), epicatechin-3-gallate (ECG), and theaflavins showed inhibition of cyclooxygenase- and lipoxygenase-dependent metabolism of arachidonic acid in human colon mucosa and colon tumor tissues (Hong et al. 2001) and induce COX-2 expression and PGE2 production (Park et al. 2001).

The studies of Acacia catechu (L.f) Willd and Scutellaria baicalensis Georgi extracts for suppressing LPS-induced pro-inflammatory responses through NF-κB, MAPK, and PI3K-Akt signaling pathways in alveolar epithelial type II cells was published recently (Feng et al., 2019). Methods for the isolation, purification and usage of compositions containing Free-B-Ring flavonoids or flavans are described in U.S. issued U.S. Pat. Nos. 9,061,039; 8,535,735; 7,972,632; and 7,192,611 entitled “Identification of Free-B-Ring Flavonoids as Potent COX-2 Inhibitors,”; and U.S. issued U.S. Pat. Nos. 9,168,242; 8,568,799; 8,124,134; 7,108,868 entitled “Isolation of a Dual COX-2 and 5-Lipoxygenase Inhibitor from Acacia”, respectively. The composition of matter of combining Free-b-Ring flavonoids and flavans and its usage for joint care, mental acuity, oral care and skin care etc. based on COX/LOX dual inhibition are described in U.S. issued U.S. Pat. Nos. 9,849,152; 9,655,940; 9,061,039; 8,535,735; 7,674,830; 7,514,469 entitled “Formulation of a mixture of Free-B-Ring flavonoids and flavans as a therapeutic agent”, U.S. issued U.S. Pat. Nos. 8,652,535; 8,034,387; 7,695,743 entitled “Formulation of a mixture of Free-B-Ring flavonoids and flavans for use in the prevention and treatment of cognitive decline and age-related memory impairments”; U.S. issued U.S. Pat. Nos. 9,622,964; 8,790,724 entitled “Formulation of dual cyclooxygenase (COX) and lipoxygenase (LOX) inhibitors for skin care”; U.S. issued U.S. Pat. No. 8,945,518 entitled “Formulation of Dual Eicosanoid System and Cytokine System Inhibitors for the Use in the Prevention and Treatment of Oral Diseases”; and U.S. issued U.S. Pat. No. 7,531,521 entitled “Formulation for prevention and treatment of carbohydrate induced diseases and conditions”, which are incorporated herein by reference in their entirety.

### SUMMARY OF THE SUBJECT MATTER

Bioflavonoid compositions for establishment and regulation of homeostasis of host defense mechanism, are disclosed and comprise at least one standardized bioflavonoid extract enriched for at least one free-B-ring flavonoid and at least one standardized bioflavonoid extract enriched for at least one flavan. Contemplated compositions are effective for respiratory diseases and conditions.

### BRIEF DESCRIPTION OF THE FIGURES

FIG. 1 shows the host defense homeostasis concept using HMGB1 as a lever for the tipping point.

FIG. 2 shows the novelty of standardized composition to maintain homeostasis of host defense mechanism.

FIG. 3 shows a schematic representation of gates (⊥) where the bioflavonoid composition may interfere the pathways of HMGB1 and NFκB.

FIG. 4 shows cell viability in 24 h hyperoxia exposure with present of UP894-II. * p<0.05 compared to room air control (Oh). #, P<0.05, ####, P<0.001, compared to vehicle control.

FIG. 5 shows UP894-II attenuates hyperoxia-compromised macrophage phagocytic function. Each value represents the mean±SEM of 2 independent experiments for each group, in duplicates. Significance is compared to the 95% O₂ (0 μg/ml) control group.

FIG. 6. UP894-II decreases the hyperoxia-induced HMGB1 release in RAW 264.7 cells. Each value represents the mean±SEM of 2 independent experiments, in duplicates. *** p<0.001 compared to room air control (RA). #p<0.05, ##P<0.01, ###P<0.001, compared to vehicle control.

FIG. 7 shows an H&E stain of lung tissue from LPS induced rats treated with UP446 at 250 mg/kg. A=normal control, B=Vehicle control, C=Sodium Butyrate, D=UP446 (250 mg/kg). Magnification 100×.

FIG. 8 shows a lung HMGB1 expression fold change of SARS-CoV-2 infected hACE2 transgenic mice.

### DETAILED DESCRIPTION

Compositions and methods are disclosed for regulation of homeostasis of host defense mechanism including a combination of one or more Free-B-Ring flavonoids from Scutellaria baicalensis with one or more flavans from Acacia catechu. Compositions for maintenance of homeostasis of host defense mechanism by regulating HMGB1, reducing oxidative stress and inducting mucosal immunity in particular production of immunoglobulins and T cells of immune and respiratory systems. Methods for treating, managing, promoting, protecting phagocytosis activity of macrophage as the first line of innate immune defense cells and providing important host defense mechanism for the population increasingly subjected to pathogenic and oxidative stress generated by air pollution, virus such as SARS-CoV-2 and microbial infections, especially for those hosts living with aging and chronic inflammatory disorders, including chronic inflammatory disorders in/of the respiratory system, in a mammal are disclosed that include administering an effective amount of a composition from 0.01 mg/kg to 500 mg/kg body weight of the mammal.

The present subject matter dictates a synergistic regulation of host defense homeostasis that leads to improved immune function, respiratory health and lung function of a host by a standardized bioflavonoid composition containing Free-B-Ring flavonoids and flavans through modulation of an extracellular protein, HMGB1, reduction of oxidative stress and induction of mucosal immunity in particular production of immunoglobulins and T cells. IgA, the second most prevalent antibody in the serum, is the first line of defense in the resistance against pulmonary and systemic infection by inhibiting microbial and viral adhesion to epithelial cells and by neutralization of bacteria, air pollutants and viruses. It should be understood that contemplated compositions do not act or perform by direct inhibition of a microbial infection or a virus to achieve the expected benefits. Contemplated embodiments regulate the homeostasis of self-defense mechanisms of the host to reduce microbial or viral infection by the defense functions of the host.

Homeostasis of the host defense mechanism has been addressed as pulmonary and systemic in the current subject matter. While the current subject matter is expected to maintain systemic mucosal homeostasis at the gastrointestinal and urogenital tracts, data depicted in the body of the subject matter confirmed its principal function in the protecting the structural integrity and function of the respiratory system primarily through modulation of HMGB1 and induction of the first line of respiratory defense mucosal immunity such as Immunoglobulin A (IgA). The pulmonary protection effect of the current subject matter was assessed on living hosts using Lipopolysaccharides (LPS)-induced acute lung injury; hyperoxia and microbial infected models in vivo; and hyperoxia-compromised macrophages in vitro. The bioflavonoid compositions containing Free-B-Ring flavonoids and flavans were tested in hyperoxia-compromised macrophage producing increased phagocytosis activity of the macrophages (an innate immune defense) by inhibiting the release of HMGB1. Substantiating these findings, in vivo, the bioflavonoid composition showed increased bacterial clearance of airways and lungs, significantly reduced the accumulation of airway HMGB1 and reduced total protein in the lungs of mice exposed to hyperoxia and microbial infection, indicating its usage in respiratory and lung protection. Similar respiratory and lung protection activities of the current subject matter were observed in the LPS-induced acute lung injury model, wherein supplementation of the bioflavonoid composition resulted in mitigation of the cardinal signs of inflammation, reduced biomarkers and lung injury. The systemic host defense homeostasis effect of the current subject matter was also assessed in Lipopolysaccharides (LPS)-induced sepsis and D-Galactose-induced accelerated aging model with and without flu vaccine immunization. In all the models tested, the current subject matter containing Free-B-Ring flavonoids and flavans showed statistically a significant improved host defense mechanism, validating its usage in restoring host defense homeostasis locally or systemically.

Bioflavonoid compositions for establishment and regulation of homeostasis of host defense mechanism, are disclosed and comprise at least one standardized bioflavonoid extract enriched for at least one free-B-ring flavonoid and at least one standardized bioflavonoid extract enriched for at least one flavan. Contemplated compositions are effective for respiratory diseases and conditions. As will be discussed herein, the at least one standardized bioflavonoid extract are enriched for at least one free-B-ring flavonoid and the at least one standardized bioflavonoid extract are enriched for at least one flavan in the composition are in a range of 1%-98% by weight of each extract with the optimized weight ratio of 80:20. Contemplated embodiments also include embodiments where the at least one standardized bioflavonoid extract enriched for at least one free-B-ring flavonoid is enriched and standardized from roots of Scutellaria baicalensis; and the at least one standardized bioflavonoid extract enriched for at least one flavan is enriched and standardized from heartwoods of Acacia catechu.

Contemplated subject matter includes bioflavonoid composition combining Free-B-Ring flavonoids and flavans showed inhibition of extracellular HMGB1 secretion locally from the lung lavage fluids and systemically from spleen homogenates in the hosts exposed to hyperoxia and microbial infection and D-Galactose induced accelerated aging models, respectively. Objective assessment of the invented composition was carried out based on key immune or inflammatory response biomarkers, such as HMGB1 and NFκB, and changes associated with immune senescence in vivo. By modulating HMGB1 and NFκB, the bioflavonoid composition containing Free-B-Ring flavonoids and flavans demonstrated a significant increase in macrophage phagocytosis in vitro and mitigation of pro-inflammatory cytokines TNF-α, IL-1β, IL-6, CRP, and CINC3, while increasing the survival rate in vivo, indicating its usage to restore, modulate and maintain homeostasis of the host defense mechanism. Similarly, the disclosed bioflavonoid composition containing Free-B-Ring flavonoids and flavans, was also found to show reversal of immune senescence as evidenced by stimulation of innate and adaptive immune responses (increased complement C3, increased CD3+ T cells, CD8+ Cytotoxic T cells, CD3-CD49b+ Natural Killer cells, NKp46+ Natural Killer cells and CD4+ TCRγδ+ Gamma delta T cells), augmentation of antioxidant capacity (decreased advanced glycation end products, increased glutathione peroxidase) and protection of key immune organs, such as thymus, from aging-associated disfunction and structural damage.

Contemplated compositions maintain immune homeostasis by optimizing or balancing the immune response; improves aging and immune organ senescence compromised immunity; prevent chronic inflammation and inflammation compromised immunity; help to maintain a healthy immune response to influenza vaccination and COVID-19 vaccination; help to maintain a healthy immune function against virus infection and bacterial infections; or protect immune system from oxidative stress damage induced by air pollution of a mammal. In addition, contemplated embodiments include a composition that regulates HMGB1 as endogenous or exogenous response assault triggers and shifts host defense response to restore homeostasis, the HMGB1 is released by immune senescence, or by inflammation, or by oxidative stress compromised immune cells; by virus, or microbial, air pollutant infected immune cells, host respiratory cells, or cardiovascular cells.

Most importantly, supplementation of the disclosed novel bioflavonoid composition containing Free-B-Ring flavonoids and flavans resulted in induction of a key mucosal defense associated immunoglobulin, IgA proven in human clinical study. IgA, the most significant antibody class present at the mucosal surface of the respiratory tract are responsible for shielding the mucosal surfaces from penetration by microorganisms and foreign antigens. The current subject matter of the bioflavonoid composition was found to statistically increase immunoglobulin IgA in a randomized double-blind placebo controlled human clinical trial as a result of supplementation of the bioflavonoid composition disclosed in the current subject matter. IgA was increased in subjects after 56 days of daily supplementation with UP446, a standardized bioflavonoid composition containing Free-B-Ring flavonoids and flavans illustrated in the current subject matter and in those who took the supplement for 56 days total with an influenza vaccination immune challenge at Day 28. Increased IgA is indicative of enhanced mucosal protection at the portal of entry at gastrointestinal, respiratory and urogenital tracts.

The merit of combining these standardized bioflavonoid extracts from two medicinal plants—Scutellaria baicalensis and Acacia catechu in the current subject matter was also tested in the LPS-induced sepsis model in vivo and unexpected synergistic effects were found as described in the body of the subject matter. In general, representing the host defense mechanism as a lever and the bioflavonoid composition containing Free-B-Ring flavonoids and flavans as a pivot point, host defense homeostasis or pulmonary protection was achieved by down modulating catabolic HMGB1 on one side of the lever and promoting the induction of mucosal immunity in particular production of (IgA), on the other.

In contemplated embodiments, the standardized bioflavonoid extracts in the composition are extracted with any suitable solvent, including supercritical fluid of CO₂, water, acidic water, basic water, acetone, methanol, ethanol, propenol, butanol, alcohol mixed with water, mixed organic solvents, or a combination thereof.

Free-B-Ring flavones and flavonols are a specific class of flavonoids, which have no substituent groups on the aromatic B ring, as illustrated by the following general structure:

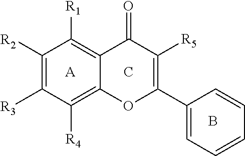

wherein

R₁, R₂, R₃, R₄, and R₅ independently comprise, and in some embodiments are selected from the group consisting of —H, —OH, —SH, OR, —SR, —NH₂, —NHR, —NR₂, —NR₃⁺X⁻, a carbon, oxygen, nitrogen or sulfur, glycoside of a single or a combination of multiple sugars including, but not limited to aldopentoses, methyl-aldopentose, aldohexoses, ketohexose and their chemical derivatives thereof;

wherein

R is an alkyl group having between 1-10 carbon atoms; and

X is selected from the group of pharmaceutically acceptable counter anions including, but not limited to hydroxyl, chloride, iodide, sulfate, phosphate, acetate, fluoride, carbonate, etc.

In contemplated embodiments, the at least one standardized bioflavonoid extract is enriched for at least one free-B-ring flavonoid comprises 0.5% to 99.5% of one or more free-B-ring flavonoids. In other embodiments, the at least one standardized bioflavonoid extract is enriched for at least one flavan comprises 0.5% to 99.5% of catechins.

In contemplated embodiments, the free-B-ring flavonoid comprises at least one of baicalin, baicalein, baicalein glycoside, wogonin, wogonin glucuronide, wogonin glycoside, oroxylin. oroxylin glycoside, oroxylin glucuronide, chrysin, chrysin glycoside, chrysin glucuronide, scutellarin and scutellarin glycoside, norwogonin, norwogonin glycoside, galangin, or a combination thereof.

The Free-B-Ring flavonoids were extracted from plants using either organic or aqueous solvent as demonstrated in the Example 1. The extraction yields are different depending on the specific species and parts of plants to be extracted with a range from low single digit to about 25% of total amount of biomass. The Free-B-ring flavonoids in the extracts can be isolated, identified and quantified with analytical methods such as UV spectrometer or PDA detector in connection with high pressure column chromatography (HPLC). The contents of Free-B-Ring flavonoids in the solvent extracts were as low as less than 1% to as high as >35% (Table 2 in Example 1). Further enrichment and standardization of the Free-B-Ring flavonoids were demonstrated in Example 2 with the targeted Free-B-Ring flavonoid content increased from about 35% from the organic solvent extract of roots of Scutellaria baicalensis to 60-90% after optimization the extraction solvent and extraction condition, neutralization of the extract solution, precipitation and filtration. RM405 was produced in the Example 2 that contained not less than 75% baicalin as the major Free-B-Ring flavonoids from the roots of Scutellaria baicalensis. The standardized bioflavonoids extract from roots or stems or whole plants of Scutellaria can be achieved by precipitation the basic aqueous extract solution after neutralization with acidic solution, or by recrystallization in water, or by column chromatography with different types of resin to achieve 2-3 folds of enrichment of bioflavonoids to a purity between 20%-99% of Free-B-Ring flavonoids.

Flavans include compounds illustrated by the following general structure:

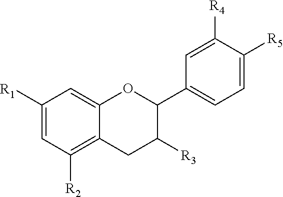

wherein

R₁, R₂, R₃, R₄ and R₅ independently comprise, and in some embodiments are selected from the group consisting of —H, —OH, —SH, —OCH₃, —SCH₃, —OR, —SR, —NH₂, —NRH, —NR₂, —NR₃⁺X⁻, esters of the mentioned substitution groups, including, but not limited to, gallate, acetate, cinnamoyl and hydroxyl-cinnamoyl esters, trihydroxybenzoyl esters and caffeoyl esters; thereof carbon, oxygen, nitrogen or sulfur glycoside of a single or a combination of multiple sugars including, but not limited to, aldopentoses, methyl aldopentose, aldohexoses, ketohexose and their chemical derivatives thereof; dimer, trimer and other polymerized flavans;

wherein

R is an alkyl group having between 1-10 carbon atoms; and

X is selected from the group of pharmaceutically acceptable counter anions including, but not limited to hydroxyl, chloride, iodide, sulfate, phosphate, acetate, fluoride, and carbonate, etc.

In some contemplated embodiments, the at least one standardized bioflavonoid extract is enriched for at least one flavan comprises at least one of catechin, epicatechin, catechingallate, gallocatechin, epigallocatechin, epigallocatechin gallate, epitheaflavin, epicatechin gallate, gallocatechingallate, theaflavin, theaflavin gallate, or a combination thereof.

Catechin is a flavan, found primarily in Acacia catechu, Uncaria gambir, Cashew nut testa, green tea, having the following structure.

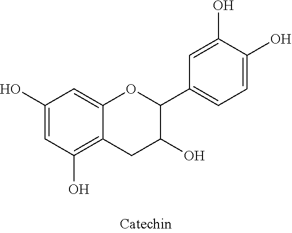 [SMILES: Oc1cc(O)c2c(c1)OC(c1ccc(O)c(O)c1)C(O)C2]

The flavan extracts were generated from different plants with organic, aqueous and alcoholic solvent extractions demonstrated in the example 3. The contents of catechin, epicatechin as of total flavans in those plant extracts were quantified by HPLC method with the results listed in the Table 4. The standardized flavan extract (RM406) from Acacia catechu heartwood was generated from aqueous extraction followed by concentration, precipitation, and recrystallization to enrich and standardize the flavan content from about 10% to 65%. The standardized bioflavonoids extracts from heartwoods, or barks or whole plants of Acacia catechu or Uncaria gambir or Cashew nut testa can be achieved by concentration of the plant extract solution, then by precipitation or by recrystallization in ethanol/water solvent, or by column chromatography with different types of resin to achieve 2-8 folds of enrichment of bioflavonoids to a purity between 10%-99% of flavans.

Example 4 demonstrated the method to make a bioflavonoid composition coded UP446 by combining two standardized extracts as Acacia extract (RM406 in example 3) contains >65% total flavans as of catechin and epicatechin with Scutellaria extract (RM405 in example 2) contains >75% Free-B-Ring flavonoids as of baicalin, baicalein and others; and with an excipient—Maltodextrin. The major and minor bio-flavonoid contents as of individual Free-B-Ring flavonoids and flavans were quantified and listed in the Table 5 with a total bioflavonoid content at 86%. Table 6 listed four different bioflavonoid compositions from different source of Free-B-Ring flavonoids such as the roots (UP446) or stems (UP223) of Scutellaria baicalensis; and different sources of flavans such as the heartwood of Acacia catechu (UP894-II) or the whole plant of Uncaria gambie (UG0408). The blending ratios of those compositions were different according to the bioflavonoid contents in each standardized extract adjusted by the intended usage and biological functionality. UP446 and UP894-II were utilized in this subject matter to disclose the unexpected synergy for the merit of combination two different types of bioflavonoids and unexpected functionality in regulation of host defense homeostasis that lead to improved immune function, protected respiratory health and lung function.

Maintain a tight host defense homeostasis is essential for physiological function of human being to defend external invasive microbial, virus, fungi, pollutants and to clear out dead cells and to initiate rebuild and renewal functions. Over stimulated immune function can cause allergic reaction and self-immune destructive diseases. Aging, oxidative stress, psychological stress, systemic inflammation, and many chronic diseases such as diabetes, obesity, metabolic syndrome can shift the host defense homeostasis tipping point leading to compromise the host defense function. Well known healthy life styles such as daily balanced nutrition, exercise, stress management and supplement with anti-oxidative, anti-inflammatory and immune regulatory (either immune suppressive or immune stimulate depends on the status of an imbalanced host defense function) natural compounds and prescriptive drugs for anti-virus, antibiotic, steroids and DTHEs can provide beneficial balance force to turn the host defense mechanism back to favorable direction. Many polyphenols including bioflavonoids were classified as immune suppressants due to the reported suppressions of cytokine productions that are essential for initiation of host defense responses to infections or vaccinations. Therefore, the real-world usage of polyphenols to support host defense mechanism has not been proven in clinical studies.

Unfortunately, there is much less knowledge and attentions paid to understand what is the tipping point that is essential for maintaining homeostasis of the host defense mechanism. Whether there is key biological, physiological and pathological pathways and biomarkers that play the role as a tipping point factor that can accelerate the shift of the host defense mechanism response to a pathological agent to a downward spiral process. Finding such a tipping point is important. More essential is whether we could find active compounds to make into a composition that can move the tipping point away from destructive direction and restore homeostasis of the host defense mechanism. We believe that HMGB1 is such biomarker that can act as an alarmin about a loss of intracellular homeostatic balance and facilitate the overwhelming biological responses under virus such as coronavirus SARS-CoV-2 and microbial infection, as well as PM2.5 pollutants that lead to compromised and destructive host defense function.

The levels of nuclear protein HMGB1 are overwhelmingly high (100 folds compared to the healthy controls) in the airways of animals and humans exposed to prolonged oxidative stress. HMGB1 was initially identified as a nuclear protein that regulates transcription, by stabilizing the structure of nucleosomes and mediating conformational changes in the DNA. In contrast to its role in the nucleus, extracellular HMGB1 induces significant inflammatory responses. Interestingly, their studies showed compiling evidence indicating that the accumulation of high levels of extracellular HMGB1 in the airways can directly compromise host defense mechanisms against bacterial and virus infections via the impairment of macrophage functions in a couple of animal models of pulmonary infections.

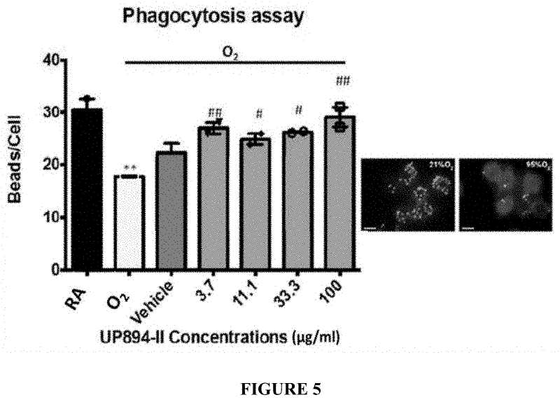

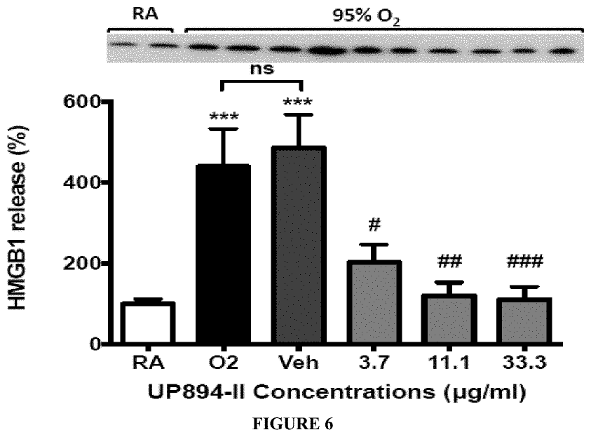

Therefore, the bioflavonoid composition UP894-II containing 70-80% Free-B-Ring flavonoids and 15-20% flavans (Table 6) was utilized to evaluate its effects on macrophages under hyperoxia stress. As shown in the Example 5, UP894-II between 8-128 μg/mL did not change macrophage viability in 24 h hyperoxia exposure (FIG. 4). UP894-II dose correlated and statistical significantly increased phagocytosis activity of macrophages at a concentration as low as 3.7 μg/mL demonstrated in the FIG. 5 of Example 6. Surprisingly, such protection of macrophage's phagocytosis activity under oxidative stress from UP894-II was closely correlated to the decreased the hyperoxia-induced HMGB1 release in Macrophages under the treatment of UP894-II with exactly same dose correlation (FIG. 6 in Example 7).

Thus, reducing the levels of HMGB1 in the airways or blocking their activities from the disclosed bioflavonoid composition UP894-II, protected phagocytosis activity of macrophage as the first line of innate immune defense cells and provided important host defense mechanism for the population increasingly subjected to pathogenic and oxidative stress generated by air pollution, virus such SARS-CoV-2 and bacterial infections, especially for those hosts living with chronic inflammatory disorders.

Objective treatment and response effects of the disclosed bioflavonoid compositions containing Free-B-Ring flavonoids and flavans were assessed in multiple in vivo studies (such as LPS induced sepsis models in Example 9-12, LPS-induced acute lung injury model in Example 13-21 and hyperoxia exposed microbial infected acute lung injury model in Example 35-39) as described in the body of the subject matter. Data depicted in those examples of this subject matter showed the significant host defense homeostatic effects of the standardized composition when administered orally in septic or acute lung injury study subjects.

The significant value of combining Free-B-Ring flavonoids from Scutellaria and Flavans from Acacia extracts was evaluated and confirmed using the commonly used Colby's equation for synergy on data obtained from the LPS induced survival study demonstrated in Example 10 and 11. With Colby's methodology, a standardized formulation with two or more materials is presumed to have unexpected synergy when the observed value is greater than the expected. In the current subject matter, it was intended to confirm the bioflavonoid composition possesses unexpected synergy for the decreased mortality rate and increased survival rate. As illustrated in Examples 12, unexpected synergy in decreasing mortality or increasing survival rate was observed from the combination of Free-B-Ring flavonoid and flavan extracts. The beneficial effects seen with the composition treatment exceeded the predicted effects based on simply summing up the effects observed for each of its constituents at the given ratio (Table 13). Only the bioflavonoid composition containing Free-B-Ring flavonoids and flavans achieved statistically significant increase in survival rate (SR %) after 144 hours of LPS challenge compared to the normal control (Table 10). In fact, 24 hours after treatment, there was no animal death (100% survival rate) observed for the bioflavonoid composition while a 15.4% and 30.8% mortality rates were observed for the Scutellaria (RM405) and Acacia (RM406) treated groups administered alone (Table 10 in Example 11), respectively. While there are reports regarding the beneficial use of these medicinal plants, however, to the best of our knowledge, this is the first-time treatment with the combination of standardized extracts from these medicinal plants resulted in unexpected outcomes in decreasing mortality rate and increasing survival rates in LPS induced sepsis. These unexpected outcomes together with other favorable innate and adaptive immune responses, in particular, the increase in IgA observed in the human clinical study as well as decreased extracellular HMGB1 documented in this subject matter, provide a unique identity to the bioflavonoid composition containing Free-B-Ring flavonoids and flavans guiding the direction of the host immune response to balanced activity resulting in overall host defense homeostasis.

Example 13 demonstrated the efficacy of a standardized bioflavonoid composition containing Free-B-Ring flavonoids and flavans on mitigating Lipopolysaccharide (LPS) induced acute inflammatory lung injury in rats. These significant changes in the level of biomarkers TNF-α (Example 14); IL-1β (Example 15) from serum, IL-6 (Example 16), CRP (Example 19), IL-10 (Example 20) and total proteins (Example 18) in broncho-alveolar lavage (BAL) and CINC-3 (Example 17) in lung homogenates as results of improved host defense homeostasis by balancing HMGB1 where later confirmed by histology examination of the lung tissues. Statistically significant reductions in the overall severity of lung damage was observed in the example 21 for animals treated with the disclosed composition. An unexpected synergistic effect was also observed in the example 11 and 12 when the merit of formulating Free-B-Ring flavonoids from Scutellaria and flavans from Acacia extracts was evaluated in the LPS induced septic model in comparison to each medicinal plant administered alone. The data from this current subject matter suggest that the bioflavonoid composition containing Free-B-Ring flavonoids and flavans help maintaining homeostasis of host defense mechanism by balancing and disrupting the vicious cycle that involve an upstream extracellular HMGB1 and subsequent NFκB signaling and cytokine storm. As a result, these key features of the composition could lead to a novel application that require a balanced host defense mechanism to protect respiratory functions from sepsis or acute or chronic injuries including but not limited to at the time of air pollution, seasonal flu or viral (e.g. COVID-19) and bacterial infections.

Instillation of LPS directly into the lung is known to activate resident innate immune response through alveolar macrophages releasing significant amount of HMGB1 leading to increased production of primary cytokines such as TNF-α, IL-1β and IL-6 as well as inflammatory protein CRP in part via activation of NFκB. These cytokines can cause significant pulmonary pathology alone or in concert triggering activation of cascades of cytokines and chemokines detrimental to disease pathology. For example, at the time of acute inflammatory response, the chemotactic cytokine induced neutrophil chemoattractant (CINC-3) which plays an important role in the recruitment of neutrophils to the lung in LPS-induced acute lung injury. Suppression of HMGB1 is the key tipping point of immune homeostasis in order to control these major cytokines and chemotactic factors involved in acute inflammatory response in the lung. Balancing HMGB1 is a key phenomenon in pulmonary pathology with significant clinical relevance in cytokine storm intervention and alleviation of severity of acute respiratory distress syndrome (ARDS).

Proteins or fibrin leakage into the interstitial space is a key component in pulmonary edema where increased exudate is an indication of disease severity. Treatment with the composition reduced total proteins from the broncho-alveolar lavage in both LPS induced acute lung injury and hyperoxia exposed and PA infected mice acute lung injury indicating its significance alleviating pulmonary pathology. These significant changes in the biomarkers from serum, BAL and homogenates have demonstrated the strategy of administering the composition to lead to a statistically significant reduction in the overall severity of lung damage that has been later confirmed by the histopathology evaluation. Based on the reduced HMGB1 level and NFκB, increased airway and lung bacterial clearance, decreased lung total protein, decreased cytokine, improved histopathology data and induced IgA depicted here, the bioflavonoid composition in deed regulates the tipping point of immune homeostasis and is indicated for cytokine storm suppression and mitigation of acute inflammatory lung injury severity.

As such, in the current subject matter, the disclosed bioflavonoid composition containing Free-B-Ring flavonoids and flavans was evaluated in the hyperoxia challenged and Pseudomonas aeruginosa (PA) infected mice in comparison with resveratrol as a positive control (Example 35). In this model, the bioflavonoid composition UP446 containing not less than 60% Free-B-Ring flavonoids and not less than 10% flavans (Table 6) was first tested for its ability in increasing survival rate of mice following a 7-day administration. Compared to the 9% mortality in mice remained in room air (RA), 64.29% mortality was observed in mice treated with hyperoxia for 2 days prior to PA inoculation (Table 36). On the other hand, mice treated with prophylactically with resveratrol (RES) and UP446 for 7 days prior to exposure to hyperoxia for 2 days and inoculated PA afterwards had mortality rate of 27.27%, and 28.57%, (Table 36) respectively. Subsequently, the bioflavonoid composition was tested and determined the effects of UP446 in reduced oxidative stress-exacerbated acute lung injury induced by pulmonary infections, using a mouse model of oxidative stress/pulmonary infection-induced acute lung injury, with PA-induced pulmonary infection and hyperoxia-induced oxidative stress (Example 36). The bioflavonoid composition containing Free-B-Ring flavonoids and flavans caused statistically significant a) reduction in the accumulation of airway HMGB1 (Table 40 in Example 39); b) increase in airway and lung bacterial clearance (Table 38 and 39 in Example 37 and 38); and c) improvement in lung injury as reflected by reduced BAL total protein (Table 37 in Example 36) in mice exposed to hyperoxia and PA infection. This correlates with the significant enhanced ability of UP446 in improving host defense against microbial infection involves the lung. In addition, UP446 improved host defense against bacterial infection in the lungs and airways. These effects play a critical role in the prevention of septic shock, and systemic inflammatory response. Data from this study highlight the benefits of the Free-B-Ring flavonoid and flavan composition—UP446 for the increasing population subjected to compromised host defense function by oxidative stress and virus or microbial infection.

Demonstrated in the example 22, in the accelerated aging model, mice were treated with D-galactose to induce an aging phenotype. After 4 weeks of D-Galactose induction, mice were treated with the disclosed Free-B-Ring flavonoid and flavan composition—UP446 at two concentrations for 4 weeks, and then introduced the influenza vaccine as an immune challenge and measured host defense mechanism in multiple assays to determine whether UP446 contributed to a balanced host defense phenotype that was similar to control mice. Significant outcomes are highlighted as:

A) In Example 23 and Table 23, The thymus indices for the normal control group and both UP446+D-Gal treatment groups were significantly higher than the D-Gal group, indicating that UP446 contributed to a reversal of thymic involution, the reduction of thymus size with age, which may affect the body's ability to mount an immune response.

B) In Example 24 and Table 24, we found significant changes in humoral immunity among the immunized groups. There was a significant increase in Complement C3 in the D-Gal+UP446 (200 mg/kg) group compared to the D-Gal alone, which indicated a prolonged humoral immune response after immunization in the UP446 treatment compared to the D-Gal group.

C) In Example 28, measuring the white blood cells in whole blood from the different groups, we found important differences among the immunized mouse groups. CD49b+(Table 28) and NKp46+ Natural Killer cells (Table 29) were increased in the immunized UP446+D-Gal groups compared to the immunized D-Gal only group. These data indicated that UP446 aided in expansion of Natural Killer cell populations, resulting in higher percentages of innate and immune cells.

D) We also found important differences among the non-immunized mouse groups. The D-Gal+UP446 groups had a strong trend toward increased CD3+ T cells (P=0.055 in Table 25), with significant increases in CD8+ Cytotoxic T cells (Table 27), NKp46+ Natural Killer cells (Table 28), CD4+ TCRγδ+ Gamma delta T cells (Table 30), and IL12p70 (Table 31) than the D-gal only group. These data demonstrated in the Examples 25-30 imply that the disclosed bioflavonoid composition UP446 primes the inactivated immune system and causes expansion of immune cell populations, increasing immune “readiness” in the non-immunized mice.

E) We examined antioxidant enzymes and biomarkers in order to surveil antioxidation pathways. The aging phenotype induced by the D-Gal model is based on an increase in Advanced Glycation End Products (AGEs), causing oxidative stress and damage, similar to the level that would be present in an older animal (Azman K F, 2019). Increasing antioxidation pathways would reduce the effects of oxidative stress. We first measured the levels of AGEs in immunized and non-immunized mouse serum samples in Example 31. We found a decrease in AGEs in mouse sera from the non-immunized D-Gal+UP446 groups (both concentrations) compared to D-gal alone (Table 32). This indicated that UP446-treated animals had lower levels of free radicals, specifically those that contributed to the aging phenotype of the D-Gal model. Next, we looked at the activity of glutathione peroxidase (GSH-Px) in mouse sera from immunized animals in example 32. We found that, compared to the immunized D-Gal group, both immunized UP446+D-Gal groups had significantly higher GSH-Px activity (Table 33), indicating an increased capacity to neutralize free radicals in the UP446-treated animals.

F) Protein levels in the spleens of animals from the immunized groups were also analyzed. The spleen is one of the main organs of the immune system. It contains a high level of white blood cells and controls the levels of immune cell types in the blood. NFκB, a pro-inflammatory transcription factor that is activated in response to inflammation, was measured in Example 33 and found that NFκB was decreased in the D-Gal+UP446 high dose treatment group (Table 34). This indicated that reducing the level of NFκB is one mechanism of UP446 to modulate inflammatory response at the time of host defense homeostasis. HMGB1, an alarmin protein that is a transcription factor and nuclear protein under non-inflammatory conditions, and which exports from the nucleus and is secreted to the extracellular space to further amplify inflammatory signals. As demonstrated in Example 34, It was found that there was a marked decrease in HMGB1 level in the non-immunized D-Gal+UP446 high dose group compared to the D-gal group (P=0.053 in Table 35). These findings all indicated that UP446 treatment reduced oxidative stress and inflammation in the non-immunized mouse spleens.

Progressive deterioration of tissues and organs reflected partly as antioxidant defense system dysfunction and immune system impairment are the hallmark of aging. Based on the free radical theory of aging, oxidative damage (the imbalance between free radicals and antioxidants) is a major contributing factor to aging and aging-associated degenerative structural and functional disorder of tissues and organs (Azman and Zakaria 2019). Elevated advanced glycation end products (AGEs) is known to accelerate the aging process and considered the main pathway for the mechanism of aging in the D-Galactose induced accelerated aging model characterized by poor immune response and disturbed antioxidant defense system. These natural occurrences were replicated in the current subject matter using a D-Galactose induced animal model where increased oxidative stress, decreased antioxidant enzyme activity and diminished immune response were observed in the D-Gal+vehicle treated mice. In contrast, supplementation of the bioflavonoid composition containing Free-B-Ring flavonoids and flavans reversed aging associated structural and functional changes. Supplementation of the bioflavonoid composition UP446 resulted in statistically significant dose-correlated reductions in serum AGE with the highest reduction being a 58% reduction in the high dose group (Table 32 in Example 31). Furthermore, the most efficient defense mechanism of cells against oxidative damage primarily involves the action of endogenous enzymatic antioxidants such as glutathione peroxidase (GSH-Px). Indeed, the bioflavonoid composition exerted potent antioxidant boosting action, with a statistically significant increase in GSH-Px for all the dosages administered (Example 33 in Example 31). Taking the induction of mucosal immunity, preservation of the immune organs, the reduction of AGEs and increased endogenous antioxidant enzymes into account, the bioflavonoid composition containing Free-B-Ring flavonoids and flavans prevents aging associated immune dysregulation and antioxidant defense system dysfunction.

Supplementation of the bioflavonoid composition to chemically-aged mice enhanced innate immunity. Activation and expansion of Natural Killer cells are key modes of immunomodulation to keep host defense homeostasis. Natural Killer cells are an important component of the innate immune system known to respond quickly to a wide variety of pathological challenges; air pollutants; viral, microbial and fungal infections; and cellular oxidative and hormonal distress, without any priming or prior activation. Natural Killer cells perform surveillance of cellular integrity to detect changes in cell surface molecules to deploy their cytotoxic effector mechanism. Natural Killer (NK) cells function as cytotoxic lymphocytes and as producers of immunoregulatory cytokines. Following stimulation, NK cells produce large amounts of cytokines, mainly gamma interferon (IFN-γ) and tumor necrosis factor (TNF-α). These cytokines and others produced by NK cells have direct effects during the early immune response and are significant modulators of the subsequent adaptive immune response, mediated through T cells and B cells. The marked increase in NK cells in the current subject matter as a result of oral administration of the bioflavonoid composition is a clear indication that the subject matter has a significant impact on innate immunity modulation, suggesting its immediate and effective immune triggering activity involved in laying a foundation for immune homeostasis. This activation of innate immunity in the form of natural killer cells is another way of the bioflavonoid composition inducing a response to protect the respiratory tract and maintain mucosal homeostasis.

Mucosal immune regulation and host defense homeostasis activities of the current subject matter have been confirmed by the level of induction observed in CD4+ TCRγδ+ Gamma delta T cells which are known for immune regulation, promoting immune surveillance and immune homeostasis. γδ T cells are a unique T cell subpopulation largely present at many portals of entry in the body, including lung and intestines, where they migrate early in their development and persist as resident cells. Due to their strategic anatomical locations (mucosal lining of the respiratory and gastrointestinal system), γδ T cells provide a first line of defense based on their innate-like responses in directly killing infected cells, recruiting other immune cells, activating phagocytosis and limiting translocation of pathogens or pollutants to the systemic compartment. These cells are known to undergo rapid population expansion and provide pathogen-specific protection on secondary challenges. Their ideal location in the respiratory and intestine tracts also helps maintain respiratory and intestinal epithelial integrity. Generally, the physiological roles of γδ T cells include protective immunity against extracellular and intracellular pathogens or pollutants, surveillance, modulation of innate and adaptive immune responses, tissue healing and epithelial cell maintenance, and regulation of physiological organ function. The γδ T cells share some characteristics with Natural Killer (NK) cells as both: are usually considered constituents of innate immunity, recognize transformed/distressed cells, play a prominent role in antiviral protection, facilitate downstream adaptive immune responses and are potent cytolytic lymphocytes. In addition, the γδ T cells assume the role of antigen presenting cells (Ribot et al., 2021; Bonneville et al., 2010). These rapidly responding immune cells (γδ T cells and the NK cells) have been induced by the bioflavonoid composition UP446 in the current subject matter leading to mucosal immune regulation, and host defense homeostasis.

Altogether, significant changes in the Free-B-Ring flavonoid and flavan composition—UP446-treated D-Gal mice were observed compared to D-Gal alone that indicated a reversion of the host defense mechanism of aging animals closer to the phenotype of the normal control mice, or at least increased host defense system priming and activation. The Thymus Indices, serum complement, Natural Killer cells, and glutathione peroxidase activity in the immunized D-Gal+UP446 groups were higher than the D-Gal alone, indicating that the host defense systems in the UP446-treated groups were better able to respond to the vaccination than the D-Gal induced aging group alone. The CD8+ Cytotoxic T cells, Natural Killer cells, and CD4+ TCRγδ+ Gamma delta T cells in the non-immunized D-Gal+UP446 groups were higher than the D-Gal alone, while the levels of AGEs, and NFkB were reduced compared to the D-Gal group, indicating both a priming of the innate and adaptive immune responses with decreased oxidative stress and inflammation. These findings show that the Free-B-Ring flavonoid and flavan composition UP446 is useful to aid in activating the host defense system both during active vaccination or infections and as a preventive to prime the host defense system against infection.

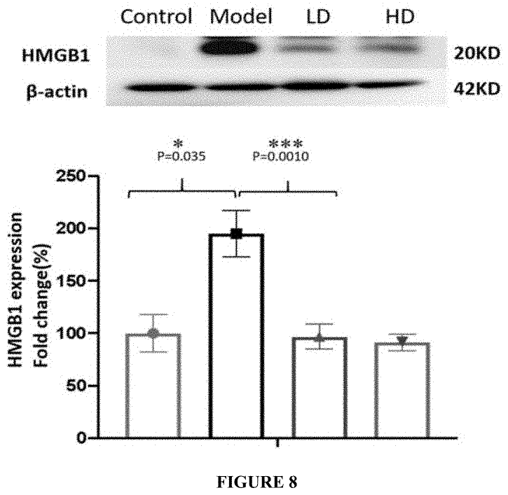

Severe acute respiratory syndrome coronavirus 2 (SARS-CoV-2) is the recently emerged RNA virus responsible for the coronavirus disease 2019 (COVID-19) pandemic with varying clinical outcomes ranging from asymptomatic infection, lung injury, inflammation, respiratory distress, multi-organ failure and death. Extracellular HMGB1 secreted in the SARS-CoV-2 infected lung has been considered as a therapeutic target in severe pulmonary inflammation of COVID-19 (Andersson et al 2020). Herbal Medicines have been considered for the treatment of SARS-CoV-2 viral attachment, acute respiratory failure and sepsis by inhibition of HMGB1 release (Wyganowska-Swiatkowska et al. 2020). Considering binding of the SARS-CoV-2 spike protein to human angiotensin I-converting enzyme 2 (hACE2) as the main portal of entry of the virus, a transgenic mouse models expressing the human ACE2 were challenged with SARS-CoV-2 for model induction and intervention efficacy. As illustrated in Example 40, vehicle treated transgenic mice infected with SARS-CoV-2 virus showed a statistically significant 2-fold increase in lung HMGB1 protein expression compared to the normal transgenic control mice without infection. In contrast, supplementation of transgenic mice infected with SARS-CoV-2 virus with a bioflavonoid composition UP894-II containing 70-80% Free-B-Ring flavonoids and 15-20% flavans resulted in the reduction of the expressions of HMGB1 protein in lung tissues to the level of the normal control transgenic mice without infection (FIG. 8). This reduction in the level of lung HMGB1 expression as a result of bioflavonoid composition treatment was statistically significant compared to vehicle treated transgenic mice infected with SARS-CoV-2. HMGB1 is a key late stage alarmin known to activate a complex sequences of host immune responses, if unchecked, leading to a cytokine storm, disturbed host defense homeostasis balance and subsequent deleterious clinical manifestations as observed in hospitalized COVID-19 patients. The marked and statistically significant reduction in the expression of HMGB1 in lung tissues in this transgenic mice infected with SRS-Cov2 indicated an improved host defense mechanism and driven homeostatic equilibrium by the bioflavonoid composition containing Free-B-Ring flavonoids and flavans that leads to a reduced cytokine storms lethality and associated lung and other organ damages caused by SARS-CoV-2 coronavirus infection.

Perhaps the most striking primary outcome for regulation of host defense mechanism from the unique bioflavonoid composition UP446 containing not less than 60% Free-B-Ring flavonoids and not less than 10% flavans was the changes of serum IgA in healthy volunteers demonstrated in Example 41 from a human clinical trial. In the double-blinded, placebo controlled clinical trial, healthy and middle age subjects (Table 42) were given daily supplementation with either UP446 250 mg twice per day or placebo for 28 days before their immune systems were challenged with an influenza vaccine (Table 41). They continued to take UP446 or placebo for an additional 28 days, with blood sample drawn for host defense biomarker measurements conducted at baseline, after 28 days of treatment, and after 56 days of treatment (28 days post-vaccination). It was found that at the end of 8 weeks treatment, mucosal immunity indicator such as immunoglobulin A was significantly increased before and after flu vaccination in subjects who received the bioflavonoid composition UP446 than placebo group. The changes in the IgA from Day 0 to Day 56 and from Day 28 to Day 56 were significantly higher for the UP446-treated group from their own inter group comparison. Through the course of the supplementation, subjects who were given the bioflavonoid composition UP446 showed marked increase in the level of IgA after influenzas vaccination compared to placebo. These data clearly show that IgA, the major immunoglobulin of healthy respiratory system and is thought to be the most important immunoglobulin for mucosal defense, is one of the main indicators of the improved homeostasis of host defense mechanism in human.

The respiratory system (i.e. the lungs and upper airways) is enriched with mucosal surface areas (400-500 m²) that are common site for frequent exposure and portal of entry to a variety of inhaled pathogens and pollutants during respiration. This continuous challenge by a large number of airborne microorganisms, microparticles, pollutants and environmental antigens requires the mucosal surfaces of the respiratory tract to engage in robust non-specific and specific defense mechanisms to protect from respiratory tract infections and injury. Besides mechanical defense (cough, sneezing, and mucociliary clearance) and removal of particles and micro-organisms by alveolar macrophages, induction of mucosal humoral immunity responses more specifically production of IgA in the respiratory tract is a crucial point for protection of respiratory system. IgA in cooperation with the non-specific innate immunity factors is considered an efficient first line of respiratory/lung defense against external agents without inducing a potentially deleterious inflammatory response. In fact, the bioflavonoid composition containing Free-B-ring flavonoids and flavans covers both the innate response by increasing macrophages phagocytosis activity and promoting adaptive response by stimulation of production of mucosal immunity in particular of IgA. IgA, the major class of immunoglobulin in the mucosa of the respiratory tract, is the most significant immunoglobulin for respiratory and lung defense known to a) shield the mucosal surfaces from penetration by microorganisms and foreign antigens, b) neutralize bacterial products; c) eliminate pathogens or antigens that have breached the mucosal surface through an IgA-mediated excretory pathway; d) agglutinate microbes and interfere with bacterial motility and e) interact with viral antigens during transcytosis and interfere with viral synthesis or assembly, thereby neutralizing viruses intracellularly (Pilette et al., 2001). As described in the body of this subject matter especially proven in human clinical trial illustrated in the example 41, supplementation with the bioflavonoid composition containing Free-B-Ring flavonoids and flavans induced mucosal immunity in particular increased production of IgA, in the human clinical trial and increased phagocytic activity of hyperoxic macrophages suggesting that the primary role of the current subject matter is protection of the lung and maintenance of mucosal immunity homeostasis.

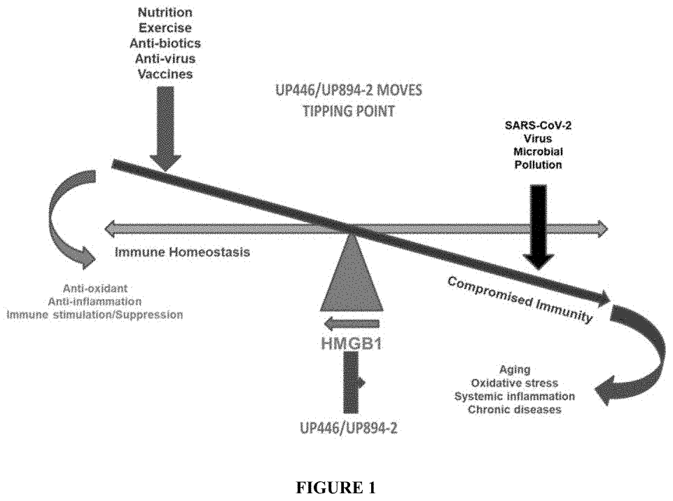

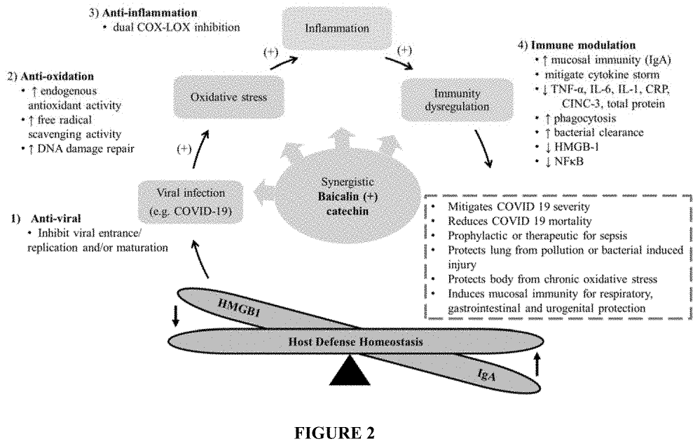

In summary, using both cell culture and animal models, it is shown that prolonged exposure to oxidative stress during oxygen therapy, which is routine used to treat patients suffer from COVID-19, can cause dramatic releases of HMGB1 that tipped the balance of immune reaction and induced the impairment of the innate immunity with compromised macrophage functions, resulting in compromised host defense to clear invading pathogens in the respiratory tracts and lungs and causing acute inflammatory of respiratory tracts and lung injury, even death. Using these model systems, HMGB1 is demonstrated as novel cellular and molecular mechanisms underlying the pathogenesis of oxidative stress-induced susceptibility to pulmonary infections and the bioflavonoid composition containing Free-B-Ring flavonoids and flavans was demonstrated to improve innate immunity and to alleviate the compromised respiratory functions by shifting HMGB1 in these hosts as demonstrated in FIG. 1 and FIG. 2. The examples of administration of the bioflavonoid composition containing Free-B-Ring flavonoids and flavans have attenuated the accumulation of extracellular HMGB1, improved respiratory functions, enhanced innate immunity against bacterial and virus infections and dampened inflammatory responses via improved homeostasis of the host defense mechanism.

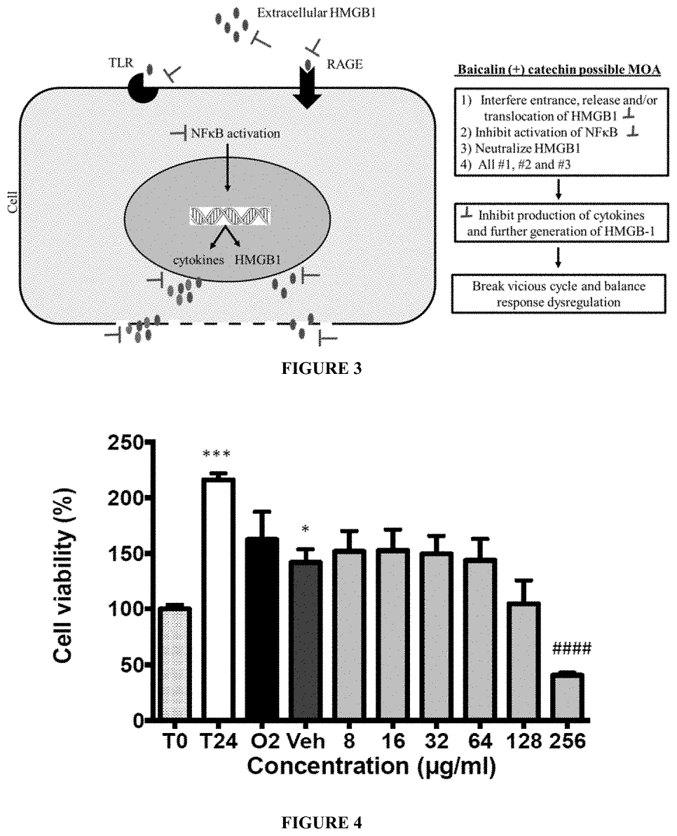

The current subject matter for modulating HMGB1 by the Free-B-Ring flavonoids and flavan can be as a result of the following but not limited to as illustrated in the FIG. 3 a) targeting HMGB1 active or passive release by blocking cytoplasm translocation, or by blocking vesicle mediated release; or inhibiting intramolecular disulfide bond formation in the nucleus b) targeting HMGB1 directly upon release and neutralize its effect c) blocking HMGB1 pattern recognizing receptors such as Toll-like Receptor (TLR)-2/4/7/9 and receptor for advanced glycation end products (RAGE) or inhibiting signal transductions. Inhibitions of oxidative stress-mediated HMGB1 release in infection, inflammation, and cell death may target the 1) CRM1-mediated nuclear export of HMGB1 in activated immune cells; 2) PARP1-mediated HMGB1 release in necrosis; 3) Caspase3/7-mediated HMGB1 release in apoptosis; 4) ATG5-mediated HMGB1 release in autophagy; 5) PKR-mediated HMGB1 release in pyroptosis; and 6) PAD4-mediated HMGB1 release in netosis. The effect of the bioflavonoid composition containing Free-B-ring flavonoids and flavans could also arise from the prevention of cluster formation or self-association of HMGB1 that could be achieved through targeting specific physiochemical factors such as ionic strength (increasing ionic strength reduces the strength of HMGB1 tetramer), pH (highest rate of self-association is at pH 4.8), metal ions especially zinc (inclusion of low dosage Zn2+ promotes HMGB1 tetramer formation), and redox environment (in a more oxidized condition, which mimics extracellular environment, HMGB1 predominantly exists as a tetramer, whereas in a more reduced condition, such as in intracellular environment, more dimer species are present). By changing the physiochemical microenvironment, the bioflavonoid composition may prevent the formation of HMGB1 tetramers and interferes in the binding affinity of HMGB1 to TLR and RAGE.

In the above and following descriptions, certain specific details are set forth in order to provide a thorough understanding of various embodiments of this disclosure. However, one skilled in the art will understand that the subject matter may be practiced without these details and limitations.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the terms “about”, “comprising”, “consisting of”, and “consisting essentially of” mean ±20% of the indicated range, value, or structure, unless otherwise indicated. It should be understood that the terms “a” and “an” as used herein refer to “one or more” of the enumerated components. The use of the alternative (e.g., “or” or “and/or”) should be understood to mean either one, both, or any combination thereof of the alternatives. Unless the context requires otherwise, throughout the present specification and claims, the word “comprise” and variations thereof, such as, “comprises” and “comprising,” as well as synonymous terms like “include” and “have” and variants thereof, are to be construed in an open, inclusive sense; that is, as “including, but not limited to.”

Reference throughout this specification to “one embodiment” or “an embodiment” means that a particular feature, structure or characteristic described in connection with the embodiment is included in at least one embodiment of the present subject matter. Thus, the appearances of the phrases “in one embodiment” or “in an embodiment” in various places throughout this specification are not necessarily all referring to the same embodiment.

The term “prodrug” is also meant to include any covalently bonded carriers, which release the active compound of this disclosure in vivo when such prodrug is administered to a mammalian subject. Prodrugs of a compound of this disclosure may be prepared by modifying functional groups present in the compound of this disclosure in such a way that the modifications are cleaved, either in routine manipulation or in vivo, to the parent compound of this disclosure. Prodrugs include compounds of this disclosure wherein a hydroxy, amino or mercapto group is bonded to any group that, when the prodrug of the compound of this disclosure is administered to a mammalian subject, cleaves to form a free hydroxy, free amino or free mercapto group, respectively. Examples of prodrugs include acetate, formate and benzoate derivatives of alcohol or amide derivatives of amine functional groups in the compounds of this disclosure and the like.

“Stable compound” and “stable structure” are meant to indicate a compound that is sufficiently robust to survive isolation to a useful degree of purity from a reaction mixture, and formulation into an efficacious therapeutic agent with a reasonable shelf life.

“Biomarker(s)” or “marker(s)” component(s) or compound(s) are meant to indicate one or multiple indigenous chemical component(s) or compound(s) in the disclosed plant(s), plant extract(s), or combined composition(s) with 2-3 plant extracts that are utilized for controlling the quality, consistence, integrity, stability, or biological functions of the invented composition(s).

“Mammal” includes humans and both domestic animals, such as laboratory animals or household pets (e.g., cats, dogs, swine, cattle, sheep, goats, horses, rabbits), and non-domestic animals, such as wildlife or the like.

“Optional” or “optionally” means that the subsequently described element, component, event or circumstances may or may not occur, and that the description includes instances where the element, component, event or circumstance occur and instances in which they do not. For example, “optionally substituted aryl” means that the aryl radical may or may not be substituted and that the description includes both substituted aryl radicals and aryl radicals having no substitution.

“Pharmaceutically or nutraceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. In contemplated embodiments, the composition further comprises a pharmaceutically or nutraceutically acceptable active, adjuvant, carrier, diluent, or excipient, wherein the pharmaceutical or nutraceutical formulation comprises from about 0.1 weight percent (wt %) to about 99.9 wt % of active compounds in the at least one standardized bioflavonoid extract.

“Pharmaceutically or nutraceutically acceptable salt” includes both acid and base addition salts. “Pharmaceutically or nutraceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecyl sulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-di sulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically or nutraceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include the sodium, potassium, lithium, ammonium, calcium, magnesium, iron, zinc, copper, manganese, aluminum salts and the like. In certain embodiments, the inorganic salts are ammonium, sodium, potassium, calcium, or magnesium salts. Salts derived from organic bases include salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly useful organic bases are isopropylamine, diethylamine, ethanolamine, trimethylamine, dicyclohexylamine, choline and caffeine.

Often crystallizations produce a solvate of the compound of this disclosure. As used herein, the term “solvate” refers to an aggregate that comprises one or more molecules of a compound of this disclosure with one or more molecules of solvent. The solvent may be water, in which case the solvate may be a hydrate. Alternatively, the solvent may be an organic solvent. Thus, the compounds of the present subject matter may exist as a hydrate, including a monohydrate, dihydrate, hemihydrate, sesquihydrate, trihydrate, tetrahydrate and the like, as well as the corresponding solvated forms. The compound of this disclosure may be true solvates, while in other cases, the compound of this disclosure may merely retain adventitious water or be a mixture of water plus some adventitious solvent.

A “pharmaceutical composition” or “nutraceutical composition” refers to a formulation of a compound of this disclosure and a medium generally accepted in the art for the delivery of the biologically active compound to mammals, e.g., humans. For example, a pharmaceutical composition of the present disclosure may be formulated or used as a standalone composition, or as a component or Active Pharmaceutical Ingredient (API) in a prescription drug, an over the counter (OTC) medicine, a botanical drug, an herbal medicine, a natural medicine, a homeopathic agent, or any other form of health care product reviewed and approved by a government agency. Exemplary nutraceutical compositions of the present disclosure may be formulated or used as a stand alone composition, or as a nutritional or bioactive component in food, a functional food, a beverage, a bar, a food flavor, a medical food, a dietary supplement, or an herbal product. A medium generally accepted in the art includes all pharmaceutically or nutraceutically acceptable carriers, diluents or excipients therefor.

As used herein, “enriched for” refers to a plant extract or other preparation having at least a two-fold up to about a 1000-fold increase of one or more active compounds as compared to the amount of one or more active compounds found in the weight of the plant material or other source before extraction or other preparation. In certain embodiments, the weight of the plant material or other source before extraction or other preparation may be dry weight, wet weight, or a combination thereof. In contemplated embodiments, the standardized bioflavonoid extracts are enriched individually or in combination by solvent precipitation, neutralization, solvent partition, ultrafiltration, enzyme digestion, column chromatograph with silica gel, XAD, HP20, LH20, C-18, alumina oxide, polyamide, ion exchange, CG161 resins, or a combination thereof.

As used herein, “major active ingredient” or “major active component” refers to one or more active compounds found in a plant extract or other preparation or enriched for in a plant extract or other preparation, which is capable of at least one biological activity. In certain embodiments, a major active ingredient of an enriched extract will be the one or more active bioflavonoid compounds that were enriched in that extract. Generally, one or more major active components in the bioflavonoid compositions will impart, directly or indirectly, most (i.e., greater than 60%, or 50%, or 20% or 10%) of one or more measurable biological activities or effects as compared to other extract components. In certain embodiments, a major active bioflavonoid may be a minor component by weight percentage of an extract (e.g., less than 50%, 25%, or 10% or 5% or 1% of the bioflavonoid contained in an extract) but still provide most of the desired biological activity. Any bioflavonoid composition of this disclosure containing a major active such as Baicalin as one of free-B-ring flavonoids may also contain minor active flavan epicatechin that may or may not contribute to the pharmaceutical or nutraceutical activity of the enriched composition, but not to the level of major active components, and minor active components alone may not be effective in the absence of a major active ingredient.

“Effective amount” or “therapeutically effective amount” refers to that amount of a bioflavonoid compound or composition of this disclosure which, when administered to a mammal, such as a human, is sufficient to shift the tipping point of host defense mechanism homeostasis that leads to the improved immune functions, including any one or more of: (1) stimulated Innate immunity (2) enhanced adaptive immunity especially CD4+ and CD8+, complement C3, increased CD3+ T cells, CD8+ Cytotoxic T cells, CD3-CD49b+ Natural Killer cells, NKp46+ Natural Killer cells and CD4+ TCRγδ+ Gamma delta T cells (3) suppressed chronic systematic inflammation and oxidative stress (4) protected immune, respiratory and lung cells from HMGB1 induced cytokine storm damage; (5) provided function as potent antioxidant to reduce oxidative stress and decrease NF-kb; decreased Advanced glycation end products, increased Glutathione Peroxidase; neutralized reactive oxygen species and prevented oxidative stress caused damage of the structural integrity and loss of function of respiratory, lung and immune system (6) maintained homeostasis of innate and adaptive immune responses; (7) enhanced phagocytic index of macrophages in humoral and cell-mediated immune responses; (8) inhibited activation of transcription factors such as NF-kB, NFAT, and STAT3; (9) inhibited lymphocyte activation and pro-inflammatory cytokines gene expression (IL-2, iNOS, TNF-α, COX-2, and IFN-γ), (10) reduced level of pro inflammatory cytokines such as IL-1β, IL-6, and TNF-α, (11) down regulated expression of COX-2, NOS-2, and NF-κB; (12) inhibited eicosanoide generation by inhibiting phospholipase A2 and TXA2 synthase activity; (13) decreased response of Th1 and Th17 cells; (14) decreased expression of ICAM and VCAM leading to decreased neutrophile chemotaxis; (15) inhibited MAPKs phosphorylation, adhesion molecules expression, signal transducers and activators of transcription 3 (STAT-3) and (16) activated transcription factor NRF2 and induce heme oxygenase-1.

Host defense function and pulmonary structure integrity and function associated “biomarkers” regulated by the compositions for regulation of homeostasis of host defense mechanism at various combinations of 2 to 3 of plant extracts with examples but not limited to UP446 or UP894-2 containing Free-B-ring flavonoids and flavans in this disclosure, include but not limited to Hemagglutinin inhibition (HI) titers for specific strains of virus, IgA, IgG, IgM, CD3+, CD4+, CD8+, CD45+, TCRγδ+, CD3-CD16+56+, GM-CSF; IFN-α; IFN-γ; IL-1α; IL-1β; IL-1RA; IL-2; IL-4; IL-5; IL-6; IL-7; IL-9; IL-10; IL-12 p70; IL-13; IL-15; IL17A; IL-18; IL-21; IL-22; IL-23; IL-27; IL-31; TNF-α; TNF-β/LTA 150, G-CSF, CCL2/3/5, IP-10, CXCL10, CRP, HMGB1, Nrf-2, INF-α/β/γ, NF-κB, PDGF-BB, MIP-1α, D-dimer, angiotensin II, cardiac troponin, VEGF, PDGF, albumin, SOD, MDA, 8-iso-prostaglandin F2a, catalase (CAT), Advanced glycation end products (AGEP), Glutathione Peroxidase, iNOS, COX1, COX2, LO5, LO12, LO13.

“Virus” as used herein include but not limited to highly pathogenic avian influenza (H5N1 virus strain A), influenza A (H1N1, H3N2, H5N1), influenza B/Washington/02/2019-like virus; influenza B/Phuket/3073/2013-like virus, Hepatitis virus A, B, C, and D; Coronavirus SARS-CoV, SARS-CoV-2 (COVID-19) MERS-CoV (MERS), Respiratory syncytial virus (RSV), Enterovirus A71 (EV71) parainfluenza, and adenovirus.

“Microbial” as used herein include but not limited to pathogenic bacterial infected respiratory system Streptococcus pneumoniae, Staphylococcus aureus, Haemophilus influenzae, Pseudomonas aeruginosa, Legionella pneumophila, and Moraxella catarrhalis are the most common bacterial pathogens; Aspergillus, Cryptococcus, Pneumocystis, Histoplasma capsulatum, Blastomyces, Cryptococcus neoformans, Pneumocystis jiroveci, Candida species (spp.) and endemic fungi that are major pulmonary fungal pathogens; in upper and lower respiratory tract infections; Streptococcus pyogenes that is the predominant bacterial pathogen in pharyngitis and tonsillitis. Bacterial infections may develop after having a viral illness like a cold or the flu.

“Respiratory and pulmonary” as used herein include but not limited to airways deliver air to the lungs and oxygen from lung to all other organs in the host such as: mouth and nose: Openings that pull air from outside host body into host respiratory system. Sinuses: hollow areas between the bones in host head that help regulate the temperature and humidity of the air host inhale; Pharynx (throat): tube that delivers air from host mouth and nose to the trachea (windpipe Trachea: Passage connecting host's throat and lungs; Bronchial tubes: tubes at the bottom of host's windpipe that connect into each lung; Lungs: Two organs that remove oxygen from the air and pass it into host blood; bloodstream delivers carbon dioxide to the lung and oxygen from the lung to all organs and other tissues of the host; muscles and bones help move the air host inhale into and out of host's lungs.

“Respiratory Infection” including the symptoms of Common cold, Stuffy, runny nose, Sneezing, Low-grade fever, headache, sore throat, pressure in the chest, wheezing, dry and raspy cough, fatigue, shortness of breath, congestion, vocal hoarseness, pain and difficult swallowing, swollen lymph nodes, Facial tenderness (specifically under the eyes or at the bridge of the nose). A few warning signs that the common cold has progressed from a viral infection to a bacterial infection include but not limited to symptoms lasting longer than 10-14 days, a fever higher than 100.4 degrees, a fever that gets worse a couple of days into the illness, rather than getting better, white pus-filled spots on the tonsils, Sinusitis with Postnasal drip, Stuffy nose/congestion, Tooth pain, Coughing, Greenish nasal discharge, Facial tenderness (specifically under the eyes or at the bridge of the nose), Bad breath, Fatigue, Fever.

“Lung infection” or “Pneumonia” is the most common bacterial or virus lower respiratory infection. It can also be caused by air pollutants, smoking tobacco, electronic tobacco or recreational marijuana. It's an infection that inflames air sacs in one or both lungs—these air sacs may fill with fluid or pus. Pneumonia symptoms include but not limited to Cough that produces phlegm or pus, Fever, Chills, Difficulty breathing, Sharp chest pain, Dehydration, Fatigue, Loss of appetite, Clammy skin or sweating, Fast breathing, Shallow breathing, Shortness of breath, Wheezing, Rapid heart rate, and drop off oxygen saturation in blood. Lung infection” or “Pneumonia” can be diagnosed by Chest X-rays, CT scan, blood tests, and culture of the sputum. The resident macrophages serve to protect the lung from foreign pathogens are triggered by inflammatory response of pathogens and are responsible for the histopathological and clinical findings seen in pneumonia. The macrophages engulf these pathogens and trigger signal molecules or cytokines like TNF-α, IL-6, and IL-1 that recruit inflammatory cells like neutrophils to the site of infection. They also serve to present these antigens to the T cells that trigger both cellular and humoral defense mechanisms, activate complement and form antibodies against these organisms. This, in turn, causes inflammation of the lung parenchyma and makes the lining capillaries “leaky,” which leads to exudative congestion and underlines the pathogenesis of pneumonia.

The amount of a compound, an extract or a composition of this disclosure that constitutes a “therapeutically effective amount” or “nutritional benefit amount” will vary depending on the bioactive compound, or nutritional component, or the biomarker for the condition being treated and its severity, the manner of administration, the duration of treatment or diet supplement, or the age of the subject to be treated, but can be determined routinely by one of ordinary skill in the art having regard to his own knowledge and to this disclosure. In certain embodiments, “effective amount” or “therapeutically effective amount” or “nutritional benefit amount” may be demonstrated as the quantity over the body weight of a mammal (i.e., 0.005 mg/kg, 0.01 mg/kg, or 0.1 mg/kg, or 1 mg/kg, or 5 mg/kg, or 10 mg/kg, or 20 mg/kg, or 50 mg/kg, or 100 mg/kg, or 200 mg/kg or 500 mg/kg). The human equivalent daily dosage can be extrapolated from the “effective amount” or “therapeutically effective amount” or “nutritional benefit amount” in an animal study by utilization of FDA guideline in consideration the difference of total body areas and body weights of animals and human.

“Dietary supplements” as used herein are a product that improves, promotes, increases, manages, controls, maintains, optimizes, modifies, reduces, inhibits, establishment, or prevents a homeostasis, a balance, a particular condition associated with a natural state or biological process, or a structural and functional integrity, an off-balanced or a compromised, or suppressed or overstimulated of a biological function or a phenotypic condition, or defense mechanism (i.e., are not used to diagnose, treat, mitigate, cure, or prevent disease). For example, with regard to host defense mechanism, “dietary supplements” may be used to modulate, maintain, manage, balance, suppress or stimulate any components of adaptive or innate immunity, as an immunoadjuvants specific to immune stimulators which enhance the efficacy of vaccine, enhance phagocytosis activity of macrophages, improve natural killing activity of NK cells, regulate level the production of proinflammatory cytokines, mitigate inflammation and tissue damage, induce response and production of antibodies, enhance antibody dependent cellular cytotoxicity, stimulate T-cell proliferation, promote the generation of immunosuppressive regulatory t-cells, and protect immune and lung cells from HMGB1 induced cytokine storm damage, check uncontrolled activation of NFκB, and protect organs or tissues from oxidative stress. In certain embodiments, dietary supplements are a special category of dietary supplement, natural nutrient, food, functional food, medical food and are not a drug.

“Treating” or “treatment” as used herein refers to the treatment of the disease or condition of interest in a mammal, such as a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving or modifying the disease or condition, i.e., causing regression of the disease or condition; or (iv) relieving the symptoms resulting from the disease or condition, (e.g., relieving cough and fever, relieving pain, reducing inflammation, reducing lung edema, mitigating pneumonia) without addressing the underlying disease or condition; (v) balancing the regulation of immunity homeostasis or changing the phenotype of the disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians. A disease or condition may be acute such as virus infection (SARS, COVID-19, MERS, Hepatitis, influenza) or microbial infection; and may be chronic such as lung damage caused by exposure to air pollution, and to smoke. A compromised immune function from off balance of homeostasis could cause a disease or a condition, or could make the mammal more susceptible infectious diseases, or could lead to more secondary organ and tissue damages directly or indirectly associated with infections from virus or microbials or air pollutants.

As used herein, “statistical significance” refers to a p value of 0.050 or less when calculated using the Students t-test and indicates that it is unlikely that a particular event or result being measured has arisen by chance.

For the purposes of administration, the compounds of the present subject matter may be administered as a raw chemical or may be formulated as pharmaceutical or nutraceutical or food compositions. Pharmaceutical or nutraceutical compositions of the present subject matter comprise a compound of structures described in this subject matter and a pharmaceutically or nutraceutically or conventional food acceptable carrier, diluent or excipient. The compound of structures described here are present in the composition in an amount which is effective to treat a particular disease or condition of interest, or supplement natural nutrients—that is, in an amount sufficient to establish homeostasis of host defense mechanism, or promote innate or adaptive immunity or immunity homeostasis in general or any of the other associated indications described herein, and generally without or with acceptable toxicity to a host.

Administration of the compounds or compositions of this disclosure, or their pharmaceutically or nutraceutically acceptable salts, in pure form or in an appropriate pharmaceutical or nutraceutical composition, can be carried out via any of the accepted modes of administration of agents for serving similar utilities. The pharmaceutical or nutraceutical compositions of this disclosure can be prepared by combining a compound of this disclosure with an appropriate pharmaceutically or nutraceutically acceptable carrier, diluent or excipient, and may be formulated into preparations in solid, semi solid, liquid or gaseous forms, such as tablets, capsules, powders, granules, ointments, solutions, beverage, suppositories, injections, inhalants, gels, creams, lotions, tinctures, sashay, ready to drink, masks, microspheres, and aerosols. The disclosed bioflavonoid composition can also be formulated into conventional food, functional food, nutritional food, medical food within other food ingredients. Typical routes of administering such pharmaceutical or nutraceutical compositions include oral, topical, transdermal, inhalation, parenteral, sublingual, buccal, rectal, vaginal, or intranasal. The term parenteral as used herein includes subcutaneous injections, intravenous, intramuscular, intrasternal injection or infusion techniques.

Pharmaceutical or nutraceutical compositions of this disclosure are formulated so as to allow the active ingredients contained therein to be bioavailable upon administration of the composition to a patient. Compositions that will be administered to a subject or patient or a mammal take the form of one or more dosage units, where for example, a tablet may be a single dosage unit, and a container of a compound or an extract or a composition of 2-3 plant extracts of this disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of this disclosure, or a pharmaceutically or nutraceutically acceptable salt thereof, for treatment of a disease or condition of interest in accordance with the teachings of this subject matter.

A pharmaceutical or nutraceutical composition of this disclosure may be in the form of a solid or liquid. In one aspect, the carrier(s) are particulate, so that the compositions are, for example, in tablet or in powder form. The carrier(s) may be liquid, with the compositions being, for example, oral syrup, injectable liquid or an aerosol, which is useful in, for example, inhalatory administration.

When intended for oral administration, the pharmaceutical or nutraceutical composition is in either solid or liquid form, where semi solid, semi liquid, suspension and gel forms are included within the forms considered herein as either solid or liquid.

As a solid composition for oral administration, the pharmaceutical or nutraceutical composition may be formulated into a powder, granule, compressed tablet, pill, capsule, chewing gum, sashay, wafer, bar, or like form. Such a solid composition will typically contain one or more inert diluents or edible carriers. In addition, one or more of the following may be present: binders such as carboxymethylcellulose, ethyl cellulose, cyclodextrin, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical or nutraceutical composition is in the form of a capsule, for example, a gelatin capsule, it may contain, in addition to materials of the above type, a liquid carrier such as polyethylene glycol or oil.

The pharmaceutical or nutraceutical composition may be in the form of a liquid, for example, an elixir, tincture, syrup, solution, emulsion or suspension. The liquid may be for oral administration or for delivery by injection, as two examples. When intended for oral administration, a useful composition contains, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical or nutraceutical compositions of this disclosure, whether they be solutions, suspensions or other like form, may include one or more of the following adjuvants: sterile diluents such as water for injection, saline solution, such as physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a generally useful adjuvant. An injectable pharmaceutical or nutraceutical composition is sterile.

A liquid pharmaceutical or nutraceutical composition of this disclosure intended for either parenteral or oral administration should contain an amount of a compound of this disclosure such that a suitable dosage will be obtained.

The pharmaceutical or nutraceutical composition of this disclosure may be intended for topical administration, in which case the carrier may suitably comprise a solution, emulsion, cream, lotion, ointment, or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical or nutraceutical composition for topical administration. If intended for transdermal administration, the composition may include a transdermal patch or iontophoresis device.

The pharmaceutical or nutraceutical composition of this disclosure may be intended for rectal administration, in the form, for example, of a suppository, which will melt in the rectum and release the drug. The composition for rectal administration may contain an oleaginous base as a suitable nonirritating excipient. Such bases include lanolin, cocoa butter and polyethylene glycol.

The pharmaceutical or nutraceutical composition of this disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical or nutraceutical composition of this disclosure in solid or liquid form may include an agent that binds to the compound of this disclosure and thereby assists in the delivery of the compound. Suitable agents that may act in this capacity include a monoclonal or polyclonal antibody, a protein or a liposome.

The pharmaceutical or nutraceutical composition of this disclosure in solid or liquid form may include reducing the size of a particle to, for example, improve bioavailability. The size of a powder, granule, particle, microsphere, or the like in a composition, with or without an excipient, can be macro (e.g., visible to the eye or at least 100 μm in size), micro (e.g., may range from about 100 μm to about 100 nm in size), nano (e.g., may no more than 100 nm in size), and any size in between or any combination thereof to improve size and bulk density.

The pharmaceutical or nutraceutical composition of this disclosure may consist of dosage units that can be administered as an aerosol. The term aerosol is used to denote a variety of systems ranging from those of colloidal nature to systems comprising pressurized packages. Delivery may be by a liquefied or compressed gas or by a suitable pump system that dispenses the active ingredients. Aerosols of compounds of this disclosure may be delivered in single phase, bi phasic, or tri phasic systems in order to deliver the active ingredient(s). Delivery of the aerosol includes the necessary container, activators, valves, sub-containers, and the like, which together may form a kit. One skilled in the art, without undue experimentation, may determine the most appropriate aerosol(s).

The pharmaceutical or nutraceutical compositions of this disclosure may be prepared by methodology well known in the pharmaceutical or nutraceutical art. For example, a pharmaceutical or nutraceutical composition intended to be administered by injection can be prepared by combining a compound of this disclosure with sterile, distilled, deionized water so as to form a solution. A surfactant may be added to facilitate the formation of a homogeneous solution or suspension. Surfactants are compounds that non covalently interact with the compound of this disclosure so as to facilitate dissolution or homogeneous suspension of the compound in the aqueous delivery system.

The compounds of this disclosure, or their pharmaceutically or nutraceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of this disclosure, or pharmaceutically or nutraceutically acceptable derivatives thereof, may also be administered simultaneously with, prior to, or after administration of food, water and one or more other therapeutic agents. Such combination therapy includes administration of a single pharmaceutical or nutraceutical dosage formulation which contains a compound or an extract or a composition with 2-3 plant extracts of this disclosure and one or more additional active agents, as well as administration of the compound or an extract or a composition with Free-B-ring flavonoids and flavans from 2-3 plant extracts of this disclosure and each active agent in its own separate pharmaceutical or nutraceutical dosage formulation. For example, a compound or an extract or a composition with 2-3 plant extracts of this disclosure and another active agent can be administered to the patient together in a single oral dosage composition, such as a tablet or capsule, or each agent can be administered in separate oral dosage formulations. Where separate dosage formulations are used, the compounds of this disclosure and one or more additional active agents can be administered at essentially the same time, i.e., concurrently, or at separately staggered times, i.e., sequentially; combination therapy is understood to include all these regimens.

It is understood that in the present description, combinations of substituents or variables of the depicted formulae are permissible only if such contributions result in stable compounds.

It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include C(O) R″ (where R″ is alkyl, aryl or arylalkyl), p methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this subject matter may not possess pharmacological activity as such, they may be administered to a mammal and thereafter metabolized in the body to form compounds of this disclosure which are pharmacologically active. Such derivatives may therefore be described as “prodrugs”. All prodrugs of compounds of this subject matter are included within the scope of this disclosure.

Furthermore, all compounds or extracts of this disclosure which exist in free base or acid form can be converted to their pharmaceutically or nutraceutically acceptable salts by treatment with the appropriate inorganic or organic base or acid by methods known to one skilled in the art. Salts of the compounds of this disclosure can be converted to their free base or acid form by standard techniques.

In any of the aforementioned embodiments, the compositions comprising mixtures of extracts or compounds may be mixed at a particular ratio by weight. For example, Scutellaria extract and Acacia extract containing bioflavonoids including but not limited to baicalin and catechin, respectively, may be blended in a 4:1 weight ratio, respectively. In certain embodiments, the ratio (by weight) of two extracts or compounds of this disclosure ranges from about 0.5:5 to about 5:0.5. Similar ranges apply when more than two extracts or compounds (e.g., three, four, five) are used. Exemplary ratios include 0.5:1, 0.5:2, 0.5:3, 0.5:4, 0.5:5, 1:1, 1:2, 1:3, 1:4, 1:5, 2:1, 2:2, 2:3, 2:4, 2:5, 3:1, 3:2, 3:3, 3:4, 3:5, 4:1, 4:2, 4:3, 4:4, 4:5, 5:1, 5:2, 5:3, 5:4, 5:5, 1:0.5, 2:0.5, 3:0.5, 4:0.5, or 5:0.5 In further embodiments, the disclosed individual Free-B-ring flavonoid extracts of Scutellaria extract and Acacia Flavan extract have been combined into a composition called UP446 as an examples but not limited to a blending ratio of 4:1.

In further embodiments, such combinations of individual extracts of Scutellaria, and Acacia at various combinations of those extracts with examples but not limited to UP446, or UP223, or UP894-II, or UG0408 were evaluated on in vitro, or ex vivo or in vivo models for advantage/disadvantage and unexpected synergy/antagonism of the perceived biological functions and effective adjustments of the homeostasis of host defense mechanism and mitigate the organ damages caused by cytokine storm, oxidative stress, and sepsis. The best compositions with specific blending ratio of individual extracts of flavans or Free-B-Ring flavonoids were selected based on unexpected synergy measured on the in vitro, or ex vivo or in vivo models due to the diversity of chemical components in each extract and different mechanism of actions from different types of bioactive flavonoid compounds in each extract, and potential enhancement of ADME of bioflavonoid compounds in the composition to maximize the biological and nutritional outputs.

In any of the aforementioned embodiments, the compositions comprising mixtures of extracts standardized with Free-B-Ring flavonoids and flavans as of bioflavonoid compounds may be present at certain percentage levels or ratios. In certain embodiments, a composition comprising an Scutellaria root extract powder or an Acacia heartwood extract can include 0.1% to 99.9% or about 10% to about 40% or about 60% to about 80% of Free-B-ring flavonoids, 0.1% to 99.9% or about 1% to about 10% or about 5% to about 50% of flavans, or a combination thereof. In certain embodiments, a composition comprising a Scutellaria Free-B-Ring flavonoid extract powder, or Acacia flavan extract can include from about 0.01% to about 99.9% baicalin or catechin or include at least 1%, 2%, 3%, 4%, 5%, 6%, 7%, 8%, 9%, 10%, 11%, 12%, 13%, 14%, 15%, 16%, 17%, 18%, 19%, 20%, 30%, 35%, 40%, 45%, 50%, 55%, 60%, 65%, 70%, 75%, 80%, 85% or 90%, 95% of baicalin or catechin.

In certain examples, a composition of this disclosure may be formulated to further comprise a pharmaceutically or nutraceutically acceptable carrier, diluent, or excipient, wherein the pharmaceutical or nutraceutical formulation comprises from about 0.05 weight percent (wt %), or 0.5 weight percent (wt %), or 5%, or 25%, or 50% or 80% to about 99 wt % of active or major active ingredients of an extract mixture. In further embodiments, the pharmaceutical or nutraceutical formulation comprises from about 0.05 weight percent (wt %) to about 90 wt % bioflavonoids, about 0.5 wt % to about 80 wt % baicalin, about 0.5 wt % to about 86 wt % total bioflavonoids, about 0.5 wt % to about 90 wt %, about 0.5 wt % to about 70 wt %, about 1.0 wt % to about 60 wt %, about 1.0 wt % to about 20 wt %, about 1.0 wt % to about 10 wt %, about 3.0 wt % to about 9.0 wt %, about 5.0 wt % to about 10 wt %, about 3.0 wt % to about 6 wt % of the major active ingredients in an extract mixture, or the like. In any of the aforementioned formulations, a composition of this disclosure is formulated as a tablet, hard capsule, soft-gel capsule, powder, or granule.

Also contemplated herein are agents of the disclosed compounds. Such products may result from, for example, the oxidation, reduction, hydrolysis, amidation, esterification, and the like of the administered compound, primarily due to enzymatic processes. Accordingly, contemplated compounds are those produced by a process comprising administering a contemplated compound or composition to a mammal for a period of time sufficient to yield a metabolic product thereof. Such products are typically identified by administering a radiolabeled or not radiolabeled compound of this disclosure in a detectable dose to an animal, such as rat, mouse, guinea pig, dog, cat, pig, sheep, horse, monkey, or human, allowing sufficient time for metabolism to occur, and then isolating its conversion products from the urine, blood or other biological samples.

Contemplated compounds, medicinal compositions and compositions may comprise or additionally comprise or consist of at least one pharmaceutically or nutraceutically or cosmetically acceptable carrier, diluent or excipient. As used herein, the phrase “pharmaceutically or nutraceutically or cosmetically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. Contemplated compounds, medicinal compositions and compositions may comprise or additionally comprise or consist of at least one pharmaceutically or nutraceutically or cosmetically acceptable salt. As used herein, the phrase “pharmaceutically or nutraceutically or cosmetically acceptable salt” includes both acid addition and base addition salts.

Contemplated Free-B-Ring-flavonoid plus flavan compositions may comprise or additionally comprise or consist of at least one additional active, adjuvant, excipient or carrier selected from one or more of Cannabis sativa full spectrum extract, CBD oil or CBD/THC, turmeric extract or curcumin, terminalia extract, willow bark extract, Aloe vera leaf gel powder, Poria coca extract, rosemary extract, rosmarinic acid, Devil's claw root extract, Cayenne Pepper extract or capsaicin, Prickly Ash bark extract, philodendra bark extract, hop extract, Boswellia extract, rose hips extract, Sophora extract, Withania somnifera, Bupleurum falcatum, Radix Bupleuri, Radix Glycyrrhiza, Fructus forsythiae, Panax quinquefolium, Panax ginseng C. A. Meyer, Korea red ginseng, Lentinula edodes (shiitake), Inonotus obliquus (Chaga mushroom), Lentinula edodes, Lycium barbarum, Phellinus linteus (fruit body), Trametes versicolor (fruit body), Cyamopsis tetragonolobus Cyamopsis tetragonolobus (guar gum), Trametes versicolor, Cladosiphon okamuranus Tokida, Undaria pinnatifida. Mentha or Peppermint extract, ginger or black ginger extract, grape seed polyphenols, Omega-3 or Omega-6 Fatty Acids, Krill oil, gamma-linolenic acid, citrus bioflavonoids, Acerola concentrate, astaxanthin, pycnogenol, resveratrol, ascorbic acid, vitamin C, vitamin D, vitamin E, vitamin K, vitamin B, vitamin A, L-lysine, calcium, manganese, Zinc, mineral amino acid chelate(s), amino acid(s), boron and boron glycinate, silica, probiotics, Camphor, Menthol, calcium-based salts, silica, histidine, copper gluconate, CMC, beta-cyclodextrin, cellulose, dextrose, saline, water, oil, UCII, shark and bovine cartilage, mushrooms, seaweeds, yeasts, brown algae, Agave Nectar, brown seaweed, fermentable fiber, cereal, sea cucumber, agave, artichokes, asparagus, leeks, garlic, onions, rye, barley kernels, wheat, pears, apples, guavas, quince, plums, gooseberries, oranges and other citrus fruits.

Contemplated Free-B-Ring-flavonoid plus flavan compositions may comprise or additionally comprise or consist of at least one additional natural phenolic active ingredient. In some embodiments, at least one bioactive ingredient may comprise or consist of plant powder or plant extract of or the like. The plant species that contain above immune suppressing natural phenolic compounds including but not limited to Piper longum Linn, Coptis chinensis Franch, Angelica sinensis (Oliv.) Diels, Toxicodendron vernicifluum, Glycyrrhiza glabra, Curcuma longa, Salvia Rosmarinus, Rosmarinus officinalis, Zingiber officinalis, Polygala tenuifolia, Morus alba, Humulus lupulus, Lonicera Japonica, Salvia officinalis L., Centella asiatica, Boswellia carteri, Mentha longifolia, Picea crassifolia, Citrus nobilis Lour, Citrus aurantium L. Camellia sinensis L. Pueraria mirifica, Pueraria lobata, Glycine max, Capsicum species, Fallopia japonica. Many phenolic compounds can also be found in various fruits and vegetables e.g. tomato, cruciferous vegetables, grapes, blueberries, raspberries, mulberries, apple, chili peppers etc.

The free-B-ring flavonoid is comprised of one or more of Baicalin, Baicalein, Baicalein glycoside, wogonin, wogonin glucuronide, wogonin glycoside, Oroxylin. Oroxylin glycoside, Oroxylin glucuronide, chrysin, chrysin glycoside, chrysin glucuronide, scutellarin and scutellarin glycoside, Norwogonin and Norwogonin glycoside, Galangin or any combination thereof. The Free-B-Ring flavonoid that can be used in accordance with the method of this subject matter include compounds illustrated by the general structure set forth above. The standardized Free-B-Ring bioflavonoids in the compositions are synthesized, metabolized, biodegraded, bioconverted, biotransformed, biosynthesized from small carbon units, by transgenic microbial, by P450 enzymes, by glycotransferase enzyme or a combination of enzymes, by microbacteria

One or more free-B-ring flavonoids are enriched and standardized from a genus of high plants comprising at least one of or a combination thereof Desmos, Achyrocline, Oroxylum, Buchenavia, Anaphalis, Cotula, Gnaphalium, Helichrysum, Centaurea, Eupatorium, Baccharis, Sapium, Scutellaria, Molsa, Colebrookea, Stachys, Origanum, Ziziphora, Lindera, Actinodaphne, Acacia, Derris, Glycyrrhiza, Millettia, Pongamia, Tephrosia, Artocarpus, Ficus, Pityrogramma, Notholaena, Pinus, Ulmus, Alpinia, or a combination thereof.

One or more free-B-ring flavonoids are enriched and standardized from a plant species comprising at least one of the following: Scutellaria baicalensis, Scutellaria barbata, Scutellaria orthocalyx, Scutellaria lateriflora, Scutellaria galericulata, Scutellaria viscidula, Scutellaria amoena, Scutellaria rehderiana, Scutellaria likiangensis, Scutellaria galericulata, Scutellaria indica, Scutellaria sessilifolia, Scutellaria viscidula, Scutellaria amoena, Scutellaria rehderiana, Scutellaria likiangensis, Scutellaria orientalis, Oroxylum indicum, Passiflora caerulea, Passiflora incarnata, Pleurotus ostreatus, Lactarius deliciosus, Suillus bellinii, chamomile, carrots, mushroom, honey, propolis, passion flowers, and Indian trumpet flower, or a combination thereof.

Flavan is comprised of one or more of catechin, epicatechin, catechingallate, gallocatechin, epigallocatechin, epigallocatechin gallate, epitheaflavin, epicatechin gallate, gallocatechingallate, theaflavin, theaflavin gallate, or any combination thereof. The flavans that can be used in accordance with the method of this subject matter include compounds illustrated by the general structure set forth above. The standardized flavan bioflavonoids in the compositions are synthesized, metabolized, biodegraded, bioconverted, biotransformed, biosynthesized from small carbon units, by transgenic microbial, by P450 enzymes, by glycotransferase enzyme or a combination of enzymes, by microbacteria.

The flavans of this subject matter are isolated from a plant or plants selected from the Acacia genus of plants. In a preferred embodiment, the plant comprises, or in some embodiments is selected from the group consisting of, or a combination thereof Acacia catechu (Black catechu), Senegalia catechu, Acacia concinna, Acacia farnesiana, Acacia Senegal, Acacia speciosa, Acacia arabica, Acacia caesia, Acacia pennata, Acacia sinuata. Acacia mearnsii, Acacia picnantha, Acacia dealbata, Acacia auriculiformis, Acacia holoserecia, Acacia mangium, Anacardium occidentale (Cashew nut testa), Uncaria gambir (White catechu), Uncaria rhynchophylla, Camellia sinensis, Camellia assumica, Euterpe oleracea (acai), Caesalpinia decapetala, Delonix regia, Ginkgo biloba, Acer rubrum, Cocos nucifera, Limonium Brasiliense, Acerola bagasse, Vitellaria paradoxa, Vitis vinifera, Lawsonia inermis, Artocarpus heterophyllus, Medicago sativa, Lotus japonicus, Lotus uliginosus, Eisenia bicyclis, Hedysarum sulfurescens, Robinia pseudoacacia; apple, apricot, prune, cherry, grape leaf, strawberry, beans, lemon, tea, black tea, green tea, red tea, barley grain, green algae (Acetabularia ryukyuensis), red algae (Chondrococcus hornemannii), Chocolate (Cocoa), green coffee beans, or a combination thereof.

In some embodiments, Free-B-ring flavonoids or flavans compounds or extracts of the present disclosure can be isolated from plant or marine sources, for example, from those plants included in the Examples and elsewhere throughout the present application. Suitable plant parts for isolation of the compounds include leaves, bark, trunk, trunk bark, stem, stem bark, twigs, tubers, root, rhizome, root bark, bark surface, young shoots, seed, fruit, androecium, gynoecium, calyx, stamen, petal, sepal, carpel (pistil), flower, stem cells or any combination thereof. In some related embodiments, the compounds or extracts are isolated from plant sources and synthetically modified to contain any of the recited substituents. In this regard, synthetic modification of the compound isolated from plants can be accomplished using any number of techniques including but not limited to total organic synthesized, metabolized, biodegraded, bioconverted, biotransformed, biosynthesized from small carbon units, by transgenic microbial, by P450 enzymes, by glycotransferase enzyme or a combination of enzymes, by microbacteria, which are known in the art and are well within the knowledge of one of ordinary skill in the art.

Other embodiments of the subject matter relate to methods of use of the standardized Free-B-Ring flavonoid plus flavan bioflavonoid composition for regulation of homeostasis of host defense mechanism at various combinations of 2 to 3 of plant extracts with examples but not limited to UP446 or UP894-II illustrated the examples in this disclosure, include but not limited to optimizing or balancing the immune response; helping to maintain a healthy immune function against virus infection and bacterial infections; protecting immune system from oxidative stress damage induced by air pollution; protecting normal healthy lung function from virus infection, bacterial infections and air pollution; supporting healthy inflammatory response; maintaining healthy level of cytokines and cytokine responses to infections; elevating and maintaining anti-inflammatory cytokines such as TNF-α, IL-1β, IL-6, GM-CSF; IFN-α; IFN-γ; IL-1α; IL-1RA; IL-2; IL-4; IL-5; IL-7; IL-9; IL-10; IL-12 p′70; IL-13; IL-15; IL17A; IL-18; IL-21; IL-22; IL-23; IL-27; IL-31; TNF-β/LTA, CRP, and CINC3; controlling oxidative response and alleviating oxidative stress; augmenting antioxidant capacity by increasing SOD and NRf2; decreasing advanced glycation end products, increasing Glutathione Peroxidase; neutralizing reactive oxygen species and preventing oxidative stress caused damage of the structural integrity and loss of function of respiratory, lung and immune system maintaining lung cleanse and detox capability; protecting lung structure integrity and oxygen exchanging capacity; maintaining respiratory passages and enhancing oxygen absorption capacity of alveoli; mitigating oxidative stress caused pulmonary damage; promoting microcirculation of the lung and protecting normal coagulation function; increasing the activity and count of the white blood cells, enhancing Natural Killer (NK) cell function; increasing the count of T and B lymphocytes; increasing CD4+ and CD8+ cell counts; increasing CD3+, CD4+ NKp46+ Natural Killer cells, TCRγδ+ Gamma delta T cells, and CD4+ TCRγδ+ Gamma delta T cells and CD8+ cell counts; protecting and promoting macrophage phagocytic activity; supporting or promoting normal antibody production; maintaining healthy pulmonary microbiota or symbiotic system in respiratory organs; relieving or reducing cold/flu-like symptoms including but not limited to body aches, sore throat, cough, minor throat and bronchial irritation, nasal congestion, sinus congestion, sinus pressure, runny nose, sneezing, loss of smell, loss of taste, muscle sore, headache, fever and chills; helping loosen phlegm (mucus) and thin bronchial secretions to make coughs more productive; reducing severity of bronchial irritation; reducing severity of lung damage or edema or inflammatory cell infiltration caused by virus infection, microbial infection and air pollution; supporting bronchial system and comfortable breathing through the cold/flu or pollution seasons; preventing or treating lung fibrosis; reducing duration or severity of common cold/flu; reducing severity or duration of virus and bacterial infection of respiratory system; preventing, or treating or curing respiratory infections caused by virus, microbial, and air pollutants; managing or treating or preventing, or reversing the progression of respiratory infections; promoting and strengthening and rejuvenating the repair and renewal function of lung and the entire respiratory system or the like.

### EXAMPLES

### Example 1. Preparation and Quantification of Free-B-Ring Flavonoids from Plants

Plant material from Scutellaria orthocalyx roots, or Scutellaria baicalensis roots or Scutellaria lateriflora whole plant was ground to a particle size of no larger than 2 mm. Dried ground plant material (60 g) was then transferred to an Erlenmeyer flask and methanol:dichloromethane (1:1) (600 mL) was added. The mixture was shaken for one hour, filtered and the biomass was extracted again with methanol:dichloromethane (1:1) (600 mL). The organic extracts were combined and evaporated under vacuum to provide the organic extract (see Table 1 below). After organic extraction, the biomass was air dried and extracted once with ultra pure water (600 mL). The aqueous solution was filtered and freeze-dried to provide the aqueous extract (see Table 1 below).

TABLE 1
Yield of Organic and Aqueous Extracts
of various Scutellaria species

| Plant Source | Amount | Organic Extract | Aqueous Extract |
|---|---|---|---|
| Scutellaria orthocalyx | 60 g | 4.04 g | 8.95 g |
| roots | | | |
| Scutellaria baicalensis | 60 g | 9.18 g | 7.18 g |
| roots | | | |
| Scutellaria lateriflora | 60 g | 6.54 g | 4.08 g |
| (whole plant) | | | |

The presence and quantity of Free-B-Ring Flavonoids in the organic and aqueous extracts from different plant species have been confirmed and are set forth in the Table 5. The Free-B-Ring Flavonoids were quantitatively analyzed by HPLC using a Luna C-18 column (250×4.5 mm, 5 μm) using 0.1% phosphoric acid and acetonitrile gradient from 80% to 20% in 22 minutes. The Free-B-Ring Flavonoids were detected using a UV detector at 254 nm and identified based on retention times by comparison with Free-B-Ring Flavonoid standards.

TABLE 2
Free-B-Ring Flavonoid Content in Active Plant Extracts

| Bioflavonoid | Weight of | % Extractible | Total amount | | % Flavonoids |
|---|---|---|---|---|---|
| Extracts | Extract | from BioMass | of Flavonoids | | in Extract |
| Scutellaria orthocalyx | 8.95 g | 14.9% | 0.2 | mg | 0.6% |
| (AE)* | | | | | |
| Scutellaria orthocalyx | 3.43 g | 5.7% | 1.95 | mg | 6.4% |
| (OE)* | | | | | |
| Scutellaria baicalensis | 9.18 g | 15.3% | 20.3 | mg | 35.5% |
| (OE)* | | | | | |
| Oroxylum indicum (OE)* | 6.58 g | 11.0% | 0.4 | mg | 2.2% |

*AE: Aqueous Extract;
*OE: Organic Extract

### Example 2. Generation of Free-B-Ring Flavonoids in Standardized Extracts of Plants

Scutellaria baicalensis roots were cleaned with water and sliced into small pieces. The cleaned and sliced roots were loaded into extractor and extracted with hot water twice at a temperature between 90-95° C. For every 1 kg of roots, about 8 L of water is added and extracted at 90-95° C. for about 1 hour. After collecting the extract solution, the roots are extracted again with 6 L/kg of water at 90-95° C. for another hour. The extract solution was collected and combined with the first extract solution. The extraction solution was filtered and then the pH of the solution was adjusted with hydrochloric acid or sulfuric acid in water to about 2. The acidic aqueous solution was standing for about 2 hours and then the precipitate was filtered and washed with purified water. The precipitated extract was dried at 80-90° C. The dried powder was grinded and blended. The extraction yield was 1 kilogram of enriched bioflavonoid extract from between 10-15 kg of roots. The contents of bioflavonoids were quantified by HPLC method as in above example 1 to produce a standardized extract coded as RM405 that contained not less than 75% baicalin with loss of dry less than 5%. The particle size of RM405 was controlled as 80% passing 80 mesh. The potential contamination of heavy metals as of lead, arsenic, Pb, Cd, and Hg were analyzed with ICP-MS. The potential contamination of coliforms, mold, yeast and total aerobic plate counts also measured to meet USP/AOAC/KFDA requirements.

The standardized bioflavonoids extract from roots, or stems or whole plants of Scutellaria can be achieved by precipitation the basic aqueous extract solution after neutralization with acidic solution, or by recrystallization in water, or by column chromatography with different types of resins to achieve 2-10 folds of enrichment of bioflavonoids to a purity between 20%-99%.

### Example 3. Development Standardized Bioflavonoid Extracts from Acacia catechu and Cashew Nut Testa

Acacia catechu (500 mg of ground bark) was extracted with the following solvent systems. (1) 100% water, (2) 80:20 water:methanol, (3) 60:40 water:methanol, (4) 40:60 water:methanol, (5) 20:80 water:methanol, (6) 100% methanol, (7) 80:20 methanol:THF, (8) 60:40 methanol:THF. The extract was concentrated and dried under low vacuum. The flavan contents in those dry extracts were quantified by HPLC method in the following with the results listed in the Table 4.

Dried ground Cashew nut testa powder (Anacardium occidentale) (60 g) were loaded into three 100 ml stainless steel tubes and extracted twice with a solvent 70% ethanol in DI water using an ASE 350 automatic extractor at 80° C. and pressure 1500 psi. The extract solution was automatically filtered and collected. The combined organic extract solution was evaporated with rotary evaporator under vacuum to give crude 70% ethanol extract (R00883-70E, 23.78 g, 39.63% extraction yield).

The following analytical method was used to determine the amount of free catechins in the bioflavonoid extracts from Acacia catechu heartwoods or Cashew nut testa by a C18 reversed-phase column (Phenomenex, USA, Luna 5 μm, 250 mm×4.6 mm) with a Hitachi HPLC/PDA system. Mobile Phase A: 0.1% phosphoric acid in water, and Mobile Phase B: acetonitrile was used for elution (Table 2) at a flow rate of 1.0 ml/min with UV absorbance at 275 nm and column temperature of 35° C. Catechin reference standards were purchased from Sigma-Aldrich Co. Reference standards were dissolved in MeOH: 0.1% H₃PO₄ (1:1) with catechin (C1251) at a concentration of 0.5 mg/ml and epicatechin (E1753) at 0.1 mg/ml. Testing samples were prepared at 2 mg/ml in 50% methanol in 0.1% H₃PO₄ in a volumetric flask and sonicated until dissolved (approximately 10 minutes), and then cooled to room temperature, mixed well and filtered through a 0.45 μm nylon syringe filter. HPLC analysis was performed by injecting a 20 μL sample into the HPLC.

TABLE 3
Gradient table of HPLC analytical method

| Time (min) | Mobile Phase A | Mobile Phase B |
|---|---|---|
| 0.0 | 85.0 | 15.0 |
| 7.0 | 85.0 | 15.0 |
| 12.0 | 10.0 | 90.0 |
| 16.5 | 10.0 | 90.0 |
| 16.6 | 85.0 | 15.0 |
| 24.0 | 85.0 | 15.0 |

The chemical components were quantified based on retention time and PDA data using catechin and epicatechin as standards. The catechins quantification results from Acacia extracts are set forth in Table 4. As shown in Table 4, the flavan extract generated from solvent extraction with 80% methanol/water provided the best concentration of flavan components. The bioflavonoid contents in the 70% ethanol extract of Cashew nut testa are 9.4% catechin and 6.1% epicatechin.

TABLE 4
Solvents for Generating Standardized Flavan Extracts from Acacia catechu

| Extraction | Weight of | % Extractible | Total amount | % Catechins |
|---|---|---|---|---|
| Solvent | Extract | from BioMass | of Catechins | in Extract |
| 100% water | 292.8 mg | 58.56% | 13 mg | 12.02% |
| water:methanol (80:20) | 282.9 mg | 56.58% | 13 mg | 11.19% |
| water:methanol (60:40) | 287.6 mg | 57.52% | 15 mg | 13.54% |
| water:methanol (40:60) | 264.8 mg | 52.96% | 19 mg | 13.70% |
| water:methanol (20:80) | 222.8 mg | 44.56% | 15 mg | 14.83% |
| 100% methanol | 215.0 mg | 43.00% | 15 mg | 12.73% |
| methanol:tetrahydrofuran | 264.4 mg | 52.88% | 11 mg | 8.81% |
| (80:20) | | | | |
| methanol:tetrahydrofuran | 259.9 mg | 51.98% | 15 mg | 9.05% |
| (60:40) | | | | |

Acacia catechu heartwoods were debarked, cleaned with water and sliced into small pieces. The cleaned and sliced heartwoods were loaded into an extractor and extracted with hot water twice at a temperature at about 115° C. For every 1 kg of catechu heartwood, about 4 L of water is added and extracted at 105-115° C. for about 5 hours. The extraction solution was filtered and then concentrated under vacuum between 50-60° C. The concentrated solution was kept cool at a temperature about 5° C. for 7-10 days and then the precipitate was filtered, and the wet cake was frozen and dried at about −20° C. for a day. The dried powder was ground, sieved and blended after drying at 90° C. for 10 hours. Extract ratio of final extract to heartwood was about 1 kg bioflavonoid extract from 20 kg catechu heartwoods. The content of bioflavonoids was quantified by HPLC method as following to produce a standardized extract coded as RA/1406 that contained not less than 65% total of catechin and epicatechin with loss of dry less than 5%. The particle size of RM406 was controlled as 80% passing 80 mesh. The potential contamination of heavy metals as of lead, arsenic, Pb, Cd, and Hg were analyzed with ICP-MS. The potential contamination of coliforms, mold, yeast and total aerobic plate counts also measured to meet USP/AOAC/KFDA requirements.

The standardized bioflavonoid extracts from heartwoods, or barks or whole plants of Acacia catechu or Uncaria gambir or Cashew nut testa can be achieved by concentration of the plant extract solution, then by precipitation or by recrystallization in ethanol/water solvent, or by column chromatography with different types of resins to achieve 2-10 folds of enrichment of bioflavonoids to a purity between 10%-99%.

### Example 4. Formulation of Standardized Bioflavonoid Compositions

A bioflavonoid composition coded UP446 was formulated with three ingredients: two standardized extracts as Acacia extract (RM406 in example 3) containing >65% total flavans as of catechin and epicatechin, Scutellaria extract (RM405 in example 2) containing >75% Free-B-Ring flavonoids as of baicalin, baicalin and others; and an excipient—Maltodextrin. The ratio of flavan and Free-B-Ring flavonoids can be adjusted based on the indications and functionality. The quantity of the excipient(s) will be adjusted based on the actual active contents in each ingredient. The blending table for each individual batch of the product has to be generated based on the product specification and QC results for each individual batch of ingredients. Excess amounts of active ingredients in the range of 2-5% is recommended to meet the product specification. Presented here is the blending table for one batch of UP446 (Lot #G1702) with the blending ratio as 80:17:3 for the extract of Free-B-Ring flavonoids:extract of Flavans:Maltodextrin.

TABLE 5
Free-B-Ring Flavonoid and Flavan
Contents in a UP446 Composition

| Active Components | Percentage Content |
|---|---|
| 1. Bioflavonoids | |
| a. Baicalin | 62.5% |
| b. Minor Bioflavonoids | |
| i. Wogonin-7-glucuronide | 6.7% |
| ii. Oroxylin A 7-glucuronide | 2.0% |
| iii. Baicalein | 1.5% |
| iv. Wogonin | 1.1% |
| v. Chrysin-7-glucuronide | 0.8% |
| vi. 5-Methyl-wogonin-7-glucuronide | 0.5% |
| vii. Scutellarin | 0.3% |
| viii. Norwogonin | 0.3% |
| ix. Chrysin | <0.2% |
| x. Oroxylin A | <0.2% |
| Total Free-B-Ring Flavonoids | 75.7% |
| 2. Flavans | |
| a. Catechin | 9.9% |
| b. Epicatechin | 0.4% |
| Total Flavans | 10.3% |
| 3. Total Bioflavonoids | 86.0% |

A bioflavonoid composition coded UP223 was formulated with standardized extract from the heartwoods of Acacia extract containing >65% total flavans as catechin and epicatechin, and the standardized extract from the stems of Scutellaria extract containing >75% Free-B-Ring flavonoids as of baicalin, baicalin and others. The blending ratio is 90:10 for the extract of Free-B-Ring flavonoids:extract of flavans.

A bioflavonoid composition coded UP894-II was formulated with standardized extract from the heartwoods of Acacia extract containing >90% total flavans as catechin and epicatechin, and the standardized extract from the roots of Scutellaria extract containing >90% Free-B-Ring flavonoids as of baicalin, baicalein and others. The blending ratio is 4:1 for the extract of Free-B-Ring flavonoids:extract of Flavans with Baicalin content between 70-80% and total catechins between 15-20% (Table 6).

TABLE 6
The illustration of three bioflavonoid compositions

| Attribute | UP446 | UP223 | UP894-II | UG0408 |
|---|---|---|---|---|
| Plant Origin | Scutellaria | Scutellaria | Scutellaria | Scutellaria |
| | baicalensis roots | baicalensis stems | baicalensis roots | baicalensis roots |
| | Acacia catechu | Acacia catechu | Acacia catechu | Uncaria gambier |
| | heartwoods | heartwoods | heartwoods | Leaves and stems |
| Extraction solvent | water | water | water | water |
| Free-B-Ring | Baicalin: ≥75.0% | Baicalin: ≥70.0% | Baicalin: ≥90.0% | Baicalin 20%-50% |
| flavonoid extract | | | | |
| Flavan extract | Catechins: ≥65.0% | Catechins: ≥65.0% | Catechins: ≥90.0% | Catechins 10%-30% |
| Composition | Baicalin: ≥60% | Baicalin: ≥60% | Baicalin 70-80% | Baicalin 10%-30% |
| Specification | | | | |
| | Catechins: ≥10% | Catechins: ≥10% | Catechins 15-20% | Catechins 1%-10% |
| Blending Ratio | 80:17:3 | 90:10 | 4:1 | 2:1 |
| | (Maltodextrin) | | | |

### Example 5: MTT Assay was Used to Determine Cell Viability in 24 Hour Hyperoxia Exposure Conditions with UP894-II

RAW 264.7 cells either remained at room air (21% oxygen O₂) or were exposed to 95% O₂ for 24 hours in the presence of UP894-II (0-256 μg/ml), a standardized bioflavonoid composition illustrated in Example 4 and Table 6, or its vehicle. Cell viability was determined by MTT assay as described by the manufacturer.

Compared to the TO control, which was the reading taken at the time of seeding, significantly more viable cells were seen in the T24 room air control group. Compared to the room air control group, cell viability in the O₂ control group (95% O₂) significantly decreased. Treatment with the vehicle, DMSO, at 0.16% and 0.32% concentrations had no effect on cell viability in O₂. To determine whether product UP894-II can improve macrophage functions that were compromised by oxidative stress, a dose curve of this product on cell viability was first carried out under either normal culturing conditions or hyperoxia conditions. The following graph (FIG. 4) is a representative result of 3 independent experiments. UP894-II at doses lower than 128 μg/ml did not significantly alter cell viability compared to the DMSO control group. Thus, UP894-II was tested for efficacy in enhancing macrophage functions at doses lower than 128 μg/ml.

### Example 6: UP894-II Increased Phagocytosis Activity of Macrophages

RAW 264.7 cells either remained at room air (21% O₂) or were exposed to 95% O₂ for 24 hours in the presence of UP894-II (0-100 μg/ml), a standardized bioflavonoid composition illustrated in Example 4 and Table 6. Cells were then incubated with FITC-labeled latex mini-beads for one hour, and stained with phalloidin and DAPI to visualize the actin cytoskeleton and nuclei, respectively. For quantification of phagocytic activity, at least 200 cells per group were counted and the numbers of beads per cell were represented as a percentage of the 21% O₂ (0 μg/ml) control group. UP894-II was tested at 3.7, 11.1, 33.3 and 100 μg/ml. These dosages were determined based on the cell viability assay.

As shown in FIG. 5, cultured macrophages were subjected to hyperoxia for 24 hours in the presence of either different concentrations of UP894-II or vehicle alone. As indicated in the images, hyperoxia exposure significantly compromised macrophage phagocytic activities. UP894-II, at doses as low as 3.7 μg/ml significantly enhanced macrophage function. These results suggest that UP894-II can be a good candidate for enhancing lung functions under oxidative stress.

### Example 7: UP894-II Decreases Hyperoxia-Induced HMGB1 Release in Macrophages

RAW 264.7 cells either remained at room air (21% O₂) or were exposed to 95% O₂ for 24 hours in the presence of UP894-II (0-33.3 μg/ml), a standardized bioflavonoid composition illustrated in Example 4 and Table 6. HMGB1 levels in the media were analyzed by Western blot analysis. The blot is the representative image of HMGB1 levels in each group, with each pair of lanes corresponding to the bar graph directly below it.

Compared to the room air control group (21% O₂), HMGB1 release in the hyperoxia control group (95% O₂) was significantly increased. The vehicle, DMSO, did not significantly alter HMGB1 release compared to the hyperoxia control group. In contrast, treatment with UP894-II resulted in dose-correlated, statistically significant reductions (75.9%-89.7%) in the level of HMGB1 when tested at 3.7 μg/ml, 11.1m/ml and 33.3 μg/ml (FIG. 6).

Particulates generated from environmental air pollution are known to exert exogenous oxidative stress to a biological system through generation of reactive oxygen species (ROS) that could lead to a compromised host defense and inflammation, subjecting to lung injury. ROS in association with HMGB1 plays a key role in lung injury pathology, causing alveolar macrophage apoptosis and decreasing alveolar macrophage phagocytosis in part through activation of NF-kB, leading to upregulation of proinflammatory cytokines and chemokines, subject to causing a cytokine storm. These factors in consortium could result in detrimental pathological changes in the lung at the time of pollution-induced lung injury, viral or bacterial infections. To present a practical example for this duo, in fact, prolonged exposure to oxidative stress during oxygen therapy, which is routinely used to treat patients suffering from COVID-19, can cause the impairment of innate immunity with reduced macrophage functions, resulting in a compromised ability to clear invading pathogens in the lungs and acute inflammatory lung injury. Thus, reducing the levels of HMGB1 in the airways or blocking their activity, may provide an important therapeutic and preventive strategy for the increasing population subjected to oxidative stress generated by cytokine storm, including COVID-19 patients, and those living with inflammatory disorders. Therefore, based on the data depicted here, UP894-II, a standardized bioflavonoid composition could be utilized for such new indications in addition to previously reported vital usages through these defined mechanisms. In the present subject matter, we demonstrated this concept and documented the effect of the standardized composition in multiple disease models as described in the subsequent examples.

### Example 8. Animals and Housing

CD-1 mice and Sprague Dawley rats were purchased from a USDA approved vendor. Eight weeks old male CD-1 mice and SD rats were purchased form Charles River Laboratories, Inc. (Wilmington, Mass.). Animals were acclimated upon arrival and used for the study. They were housed in a temperature-controlled room (71-72° F.) on a 12-hour light-dark cycle and provided with feed and water ad libitum.

The animals were housed 3-5 per polypropylene cage and individually identified by characteristically numbered on their tail. Each cage was covered with mouse or rat wire bar lid and filtered top (Allentown, N.J.). Individual cages were identified with a cage card indicating project number, test article, dose level, group, animal number and sex. The Harlan T7087 soft cob beddings was used and changed at least twice/week. Animals were provided with fresh water and rodent chow diet #T2018 from Harlan (Harlan Teklad, 370W, Kent, Wash.) ad libitum.

### Example 9: Lipopolysaccharide (LPS)-Induced Sepsis Model

This model used survival rate of animals as the end point measurement (Wang et al., 1999). Lipopolysaccharide (LPS) is an integral component of the outer membrane of gram-negative bacteria and a major contributing factor in the initiation of a generalized inflammatory process that may lead to endotoxin shock. It is a state mediated principally by macrophages/monocytes and is attributed to excessive production of several early phase cytokines such as TNF-α, IL-1, IL-6 and gamma interferon (IFN-γ) as well as a late-stage mediator, HMGB1. Following a median lethal dose of LPS (25 mg/kg) administration dissolved in phosphate-buffered saline (PBS; Lifeline, Lot #07641), animals develop endotoxemia and HMGB1 would be detected in the serum at 8 hours and reach to a peak and plateau levels from 16 to 32 hours after LPS. If untreated, mice would start to die within 24 hours. In the current study, we monitored the mice for 4 days after LPS injection. The survival rate compared LPS+sodium butyrate (SB; Aldrich, St. Louis, Mo.; lot #MKCG7272), LPS+Vehicle (0.5% CMC; Spectrum, New Brunswick, N.J.; lot #1IJ0127) and LPS+UP446, the standardized bioflavonoid composition illustrated in Example 4 and Table 6. The following groups were included in the study:

TABLE 7
Details of Treatment groups

| Group | Treatment | Dose (mg/kg) | N |
|---|---|---|---|
| G1 | Normal control | 0 | 8 |
| G2 | Vehicle control (0.5% CMC) | 0 | 8 |
| G3 | Sodium Butyrate (SB) | 500 | 8 |
| G4 | UP446 | 250 | 8 |

In this model, mice were pretreated with bioflavonoid composition—UP446, illustrated in the Example 4, for a week (7 days) before lethal dose intraperitoneal injection of LPS (E. coli, 055:B5; Sigma, St. Louis, Mo.; Lot #081275) at 25 mg/kg with a 10 mL/kg PBS volume. Animals were observed hourly. Given the fact that sodium butyrate improved LPS-induced injury in mice through suppression of HMGB1 release, we chose this compound as a positive control for our study (Li et al., 2018).

### Example 10: A Standardized Bioflavonoid Composition Improved Animal Survival Rate Under Lethal Dose of Endotoxin

Three hours following intraperitoneal injection of LPS, mice started to show early signs of endotoxemia. Exploratory behavior of mice was progressively reduced and was accompanied by ruffled fur (piloerection), decreased mobility, lethargy, and diarrhea. While these signs and symptoms seemed to be present in all the treatment groups, the magnitude of severity was more pronounced in the vehicle-treated group.

Two mice from the vehicle-treated and one mouse from the positive control, sodium butyrate (SB), groups were found deceased 24 hours after LPS injection. The survival rates were determined for these groups and were found as 62.5% and 75%, respectively (Table 8). Mice treated with UP446, a standardized bioflavonoid composition illustrated in Example 4 and Table 6, had a 100% survival rate after 24 hours of LPS injection. A survival rate of 87.5%, 62.5% and 50% were observed for mice treated with UP446, SB and vehicle, respectively, 34 hours after LPS injection. Perhaps the most significant observation for UP446 treated mice was observed 48 hours after LPS injection. At this time point, there was only 12.5% survival rate for the vehicle-treated mice while UP446-treated mice showed a 75% survival rate. Even for the positive control, sodium butyrate, group, half of the animals were deceased at this time point. On the third day (72 hours after LPS injection), the survival rates for the groups were 62.5%, 50% and 12.5% for UP446, SB and vehicle, respectively. All mice in the vehicle group were deceased after 82 hours of LPS injection, leaving 0% survival rate for this group.

On the other hand, mice treated with UP446 and SB showed a 50% survival rate and remained the same for 96 hours and 120 hours after LPS injection. These survival rates were statistically significant for both UP446 (p=0.001) and SB (p=0.01) when compared to the vehicle-treated animals (Table 8). Surviving animals in these groups showed progressive improvements in their wellbeing. Mice appeared physically better and gradually resumed to show normal behaviors.

TABLE 8
UP446 provided a 50% survival rate from LPS-induced endotoxemia and sepsis

| | | | | | | | | | | Survival | |
|---|---|---|---|---|---|---|---|---|---|---|---|
| | | | | | | | | | | Rate (%) | P- |
| | | # of Death after LPS | | | | | | | | after 82 hr | values |
| Group | N | 24 hr | 32 hr | 34 hr | 48 hr | 58 hr | 72 hr | 82 hr | Total | — | — |
| Control | 8 | 0 | 0 | 0 | 0 | 0 | 0 | 0 | 0 | 100 | — |
| Vehicle | 8 | 3 | 4 | 4 | 7 | 7 | 7 | 8 | 8 | 0 | — |
| UP446 | 8 | 0 | 1 | 1 | 2 | 2 | 3 | 4 | 4 | 50 | 0.00109 |
| Sodium | 8 | 2 | 3 | 3 | 4 | 4 | 4 | 4 | 4 | 50 | 0.01481 |
| Butyrate | | | | | | | | | | | |

The survival rate was calculated as: 100-[(deceased mice/total number of mice)×100]%.

### Example 11: Comparison of the Standardized Bioflavonoid Composition and its Constituents in the LPS-Induced Sepsis Model

The merit of combining Free-B-Ring flavonoids from Scutellaria extract and Flavans from Acacia extract to yield UP894-II at a specific ratio demonstrated in Example 4 was evaluated in Lipopolysaccharide (LPS)-induced endotoxemia. Male CD-1 mice (n=13) were treated with Scutellaria extract, RM405, containing not less than 60% Baicalin, illustrated in example 3, and Acacia extract, RM406, containing not less than 10% catechins, illustrated in example 4, at 200 mg/kg and 50 mg/kg, respectively, for 7 days before LPS injection. On the 8^{th} day, mice were injected intraperitoneally (i.p.) with 25 mg/kg LPS dissolved in PBS at 10 mL/kg. Mice in the UP894-II-treated group received a daily dose of UP894-II at 250 mg/kg. All mice continued to receive the treatment daily for the duration of study, which was completed on the 6^{th} day post LPS injection. Following a median lethal dose of LPS (25 mg/kg) by i.p. administration, animals are expected to develop sepsis within a few hours. If untreated, mice would start to die within 24 hours. Animals were observed hourly. In the current study, we monitored the mice for 6 days after LPS injection.

TABLE 9
Details of Treatment groups

| Group | Treatment | Dose (mg/kg) | N |
|---|---|---|---|
| G1 | Normal control | 0 | 13 |
| G2 | Vehicle control (0.5% CMC) | 0 | 13 |
| G3 | Sodium Butyrate (SB) | 500 | 13 |
| G4 | UP894-II (RM405 + RM406) | 250 | 13 |
| G5 | Scutellaria baicalensis Ext. (RM405) | 200 | 13 |
| G6 | Acacia catechu Ext. (RM406) | 50 | 13 |

The survival rate compared LPS+sodium butyrate (SB), LPS+vehicle (0.5% CMC), LPS+UP894-II, LPS+Scutellaria extract (RM405) and LPS+Acacia extract (RM406). Normal control animals received only PBS i.p. and were gavaged only with the carrier vehicle, 0.5% CMC. Given the fact that sodium butyrate (SB) improved LPS-induced injury in mice through suppression of HMGB1 release, we chose this compound as a positive control for our study (Li et al., 2018).

The survival rate and mortality rate of the composition (UP894-II) was compared with those dosages of individual extracts as they appeared in the formulation to find out potential additive, antagonist or synergistic effects in combination using Colby's equation (Colby, 1967). For the blending of these plant extracts to have unexpected synergy, the observed inhibition needs to be greater than the calculated value.

Few hours post intraperitoneal injection of LPS, mice started to show early signs of sepsis. Exploratory behavior of mice was progressively reduced and was accompanied by ruffled fur (piloerection), decreased mobility, lethargy, diarrhea, and shivering, accompanied by closed eye lids for some. While these signs and symptoms were present in all the treatment groups, the magnitude of severity was more pronounced in the vehicle and Acacia extract (RM406)-treatment groups.

Four mice from the vehicle-treated and Acacia extract (RM406 illustrated in example 4); and two mice from the positive control, SB, and Scutellaria extract (RM405 illustrated in example 3) groups were found deceased 24 hours after LPS injection. The survival rates were determined for these groups at this time point and were found as 69.2% for the vehicle and Acacia extract (RM406) and 84.6% for Scutellaria extract (RM405) and SB (Table 10). Mice treated with UP894-II had a 100% survival rate after 24 hours of LPS injection. Survival rates of 84.6%, 61.5%, 53.9%, 53.9% and 53.9% were observed for mice treated with UP894-II, Scutellaria extract (RM405), vehicle, SB and Acacia extract (RM406) respectively, 36 hours after LPS injection. The most significant observation for UP894-II-treated mice was noticed 48 hours after LPS injection where there was only a 15.4% survival rate for the vehicle-treated mice while UP894-II-treated mice showed a 69.2% survival rate. Mice treated with Scutellaria extract (RM405), Acacia extract (RM406) and SB showed 46.2%, 38.5% and 46.2% survival rates at 48-hours post LPS, respectively.

On the third day (72 hours after LPS injection), the survival rates for the treatment groups were 53.9%, 30.8%, 15.4% and 46.2% for UP894-2, Scutellaria extract (RM405), Acacia extract (RM406) and SB, respectively.

TABLE 10
Time course of survival and mortality in LPS-induced sepsis

| | Dose | | Number of deceased animals post LPS (hours) | | | | | | | | | | MR | SR |
|---|---|---|---|---|---|---|---|---|---|---|---|---|---|---|
| Group | (mg/kg) | N | 24 | 36 | 48 | 60 | 72 | 96 | 120 | 144 | Deceased | Survived | (%) | (%) |
| Control | 0 | 13 | 0 | 0 | 0 | 0 | 0 | 0 | 0 | 0 | 0 | 13 | 0.0 | 100.0 |
| Vehicle | 0 | 13 | 4 | 2 | 5 | 0 | 0 | 0 | 0 | 0 | 11 | 2 | 84.6 | 15.4 |
| SB | 500 | 13 | 2 | 4 | 1 | 1 | 0 | 1 | 0 | 0 | 9 | 4 | 69.2 | 30.8 |
| UP894-II | 250 | 13 | 0 | 2 | 2 | 1 | 1 | 0 | 0 | 0 | 6 | 7 | 46.2 | 53.9* |
| RM405 | 200 | 13 | 2 | 3 | 2 | 1 | 1 | 0 | 0 | 0 | 9 | 4 | 69.2 | 30.8 |
| RM406 | 50 | 13 | 4 | 2 | 2 | 1 | 2 | 1 | 0 | 0 | 12 | 1 | 92.3 | 7.7 |

The survival rate was calculated as: 100-[(deceased mice/total number of mice)×100]%. *p≤0.05

TABLE 11
Survival rate of LPS-induced septicnuce

| | Dose | Survival rate | | | | | | | | |
|---|---|---|---|---|---|---|---|---|---|---|
| Group | (mg/kg) | 0 hr | 24 hr | 36 hr | 48 hr | 60 hr | 72 hr | 96 hr | 120 hr | 144 hr |
| Control | 0 | 100 | 100 | 100 | 100 | 100 | 100 | 100 | 100 | 100 |
| Vehicle | 0 | 100.0 | 69.2 | 53.8 | 15.4 | 15.4 | 15.4 | 15.4 | 15.4 | 15.4 |
| SB | 500 | 100.0 | 84.6 | 53.8 | 46.2 | 38.5 | 38.5 | 30.8 | 30.8 | 30.8 |
| UP894-II | 250 | 100.0 | 100.0 | 84.6 | 69.2 | 61.5 | 53.9 | 53.9 | 53.9 | 53.9 |
| RM405 | 200 | 100.0 | 84.6 | 61.5 | 46.2 | 38.5 | 30.8 | 30.8 | 30.8 | 30.8 |
| RM406 | 50 | 100.0 | 69.2 | 53.9 | 38.5 | 30.8 | 15.4 | 7.7 | 7.7 | 7.7 |

The survival rate was calculated as: 100 − [(deceased mice/total number of mice) × 100]%.

TABLE 12
Mortality rate of LPS-induced septic mice

| | Dose | Mortality rate | | | | | | | | |
|---|---|---|---|---|---|---|---|---|---|---|
| Group | (mg/kg) | 0 hr | 24 hr | 36 hr | 48 hr | 60 hr | 72 hr | 96 hr | 120 hr | 144 hr |
| Control | 0 | 0.00 | 0.00 | 0.00 | 0.00 | 0.00 | 0.00 | 0.00 | 0.00 | 0.00 |
| Vehicle | 0 | 0.00 | 30.77 | 46.15 | 84.62 | 84.62 | 84.62 | 84.62 | 84.62 | 84.62 |
| SB | 500 | 0.00 | 15.38 | 46.15 | 53.85 | 61.54 | 61.54 | 69.23 | 69.23 | 69.23 |
| UP894-II | 250 | 0.00 | 0.00 | 15.38 | 30.77 | 38.46 | 46.15 | 46.15 | 46.15 | 46.15 |
| RM405 | 200 | 0.00 | 15.38 | 38.46 | 53.85 | 61.54 | 69.23 | 69.23 | 69.23 | 69.23 |
| RM406 | 50 | 0.00 | 30.77 | 46.15 | 61.54 | 69.23 | 84.62 | 92.30 | 92.30 | 92.30 |

The Mortality rate was calculated as: 100 − survival rate.

The survival rate for the vehicle-treated mice remained at 15.4% for the rest of the study period as of 48-hours post-LPS injection. In contrast, Acacia extract (RM406)-treated mice continued to die until the 96 hours post-LPS injection. By the end of the 7-day observation period, there was only a 7.7% survival rate for the Acacia extract (RM406) group. On the other hand, mice treated with UP894-II and Scutellaria extract (RM405) maintained 53.9% and 30.8% survival rates, respectively, as of the 3rd day post-LPS injection and for the remainder of the observation period. The positive control, sodium butyrate (SB), group finished the study with a 30.8% survival rate. When compared to the vehicle control, only the UP894-II group survival rate was statistically significant (p=0.01). Surviving animals in the groups showed progressive improvements in their wellbeing. Mice appeared physically better and gradually resumed to show normal exploratory behaviors.

### Example 12: Unexpected Synergy was Observed for the Standardized Bioflavonoid Composition

The LPS-induced survival study was utilized to evaluate possible synergy or unexpected effects of extracts from Scutellaria and Acacia, when formulated together in a specific ratio, using Colby's method. When mice were given UP894-II, a standardized bioflavonoid composition illustrated in Example 4 and Table 6, at a dose of 250 mg/kg, the survival rates were greater than the theoretically calculated expected values at each time points analyzed (Table 13). For example, while the expected survival rates at 24 and 144 hours post-LPS injection were 95.3% and 36.1%, respectively, the actual observed survival rates for UP894-II were 100% and 53.9%, respectively. These findings suggest that combining two standardized Free-B-Ring flavonoid and flavan extracts from Scutellaria and Acacia at a specific ratio has a far greater benefit than using either Acacia or Scutellaria extract alone in prolonging the life of study subjects at the time of sepsis. Using the same Colby's method, we also determined what would have been the expected mortality rate for those time points and we found that the observed mortality rates for the UP894-II treated mice were far less than predicted, confirming a better survival prognosis for these subjects as a result of the combination therapy (Table 13).

For instance, at 24 hours post LPS injection, the expected mortality rate was 41.4%, in fact there was no death for the UP894-II treated mice. It was also expected that 97.6% of the study subjects would be deceased at the end of the observation period, whereas the actual mortality rate for the UP894-II was only 46.2%. As such, in this survival study, the merit of combining Scutellaria and Acacia extracts was evaluated using Colby's equation. In this method, a formulation with two bioflavonoid extracts is presumed to have unexpected synergy if the observed value of a certain endpoint measurement is greater than the hypothetically calculated expected values.

TABLE 13
Unexpected Synergy was observed for the bioflavonoid composition UP894-II

| | Survival rate (%) | | | | Mortality rate (%) | | | |
|---|---|---|---|---|---|---|---|---|
| Hours | | | | Observed | | | | Observed |
| post LPS | X | Y | Expected | (UP894-2) | X | Y | Expected | (UP894-2) |
| 24 | 84.6 | 69.2 | 95.3 | 100 | 15.4 | 30.8 | 41.0 | 0.0 |
| 36 | 61.5 | 53.9 | 82.3 | 84.6 | 38.5 | 46.2 | 66.9 | 15.4 |
| 48 | 46.2 | 38.5 | 66.9 | 69.2 | 53.9 | 61.5 | 82.3 | 30.8 |
| 60 | 38.5 | 30.8 | 57.4 | 61.5 | 61.5 | 69.2 | 88.2 | 38.5 |
| 72 | 30.8 | 15.4 | 41.4 | 53.9 | 69.2 | 84.6 | 95.3 | 46.2 |
| 96 | 30.8 | 7.7 | 36.1 | 53.9 | 69.2 | 92.3 | 97.6 | 46.2 |
| 120 | 30.8 | 7.7 | 36.1 | 53.9 | 69.2 | 92.3 | 97.6 | 46.2 |
| 144 | 30.8 | 7.7 | 36.1 | 53.9 | 69.2 | 92.3 | 97.6 | 46.2 |

X = RM405, Y = RM406; Colby's equation for Expected survival rate: (X + Y) − (XY/100)

Survival and mortality rate values of Scutellaria extract (RM405 illustrated in example 3) (200 mg/kg) and Acacia extract (RM406 illustrated in example 4) (50 mg/kg) at 24, 36, 48, 60, 72, 96, 120 and 144 hours after LPS injection were used to determine the calculated survival and mortality rates and compared to the observed survival rate values of the composite UP894-II (250 mg/kg) at the specified time points. In the present study, we found unexpected synergy in the combination of Scutellaria extract (RM405) with Acacia extract (RM406). The beneficial effects of UP894-II treatment exceeded the sum of the effects of its constituents for all the time points examined. At the end of the observation period (i.e. 7 days after LPS injection and 14 days after oral administration of the extracts and the composition), there were 53.9%, 30.8% and 7.7% survival rates for UP894-II, Scutellaria extract (RM405) and Acacia extract (RM406) treatment groups, respectively, suggesting the unexpected synergistic activities of these botanical extracts in protecting hosts from a cytokine storm and hence increasing survival rate of patients at the time of sepsis.

### Example 13: Efficacy of a Standardized Bioflavonoid Composition on Mitigating Lipopolysaccharide (LPS)-Induced Acute Inflammatory Lung Injury in Rats—Study Design

The study was designed to evaluate the direct impact of the bioflavonoid composition UP446 contain Free-B-Ring flavonoids and flavans illustrated in Example 4 in alleviating LPS-induced acute lung injury administered orally at 250 mg/kg (High dose) and 125 mg/kg (Low dose). Acute lung injury is a clinical syndrome caused by alveolar epithelial cell and capillary endothelial cell damage, resulting in diffuse lung injury as seen in acute respiratory distress syndrome (ARDS). In this study, we treated Sprague Dawley rats with the test materials orally for 7 days before model induction with LPS. On the 8^{th} day, an hour after oral treatment, LPS was instilled intratracheally (i.t.) at 10 mg/kg dissolved in 0.1 mL/100 g PBS to each rat. The normal control rats received the same volume i.t. of PBS only.

TABLE 14
Study groups

| Group | Treatment | Dose (mg/kg) | N |
|---|---|---|---|
| G1 | Normal control | 0 | 7 |
| G2 | Vehicle control | 0 | 10 |
| G3 | Sodium Butyrate | 500 | 10 |
| G4 | UP446 -High dose | 250 | 10 |
| G5 | UP446-Low dose | 125 | 10 |

LPS is known to induce systemic and pulmonary responses, leading to accumulation of proinflammatory immune cells, including neutrophils and macrophages, and proinflammatory cytokines, such as IL-1, IL-8, IL-6, MIP-2/CINC-3 and TNF-α, causing pulmonary interstitial, alveolar edema and epithelial cell damage where HMGB1 is secreted actively by macrophages and monocytes or passively released from necrotic cells.

We sacrificed surviving animals 24 hours after intratracheal LPS administration. At necropsy, the bronchoalveolar lavage (BAL) was collected by intratracheal injection of 1.5 mL PBS into the right lobe of the lung, followed by a gentle aspiration at least 3 times. Pooled, recovered fluid was centrifuged at 1,500 rpm for 10 min at 4° C., and was used to measure cytokines (e.g. IL-6) and pulmonary protein levels. This same right lobe was collected for tissue homogenization from each rat for MIP-2/CINC-3 activity analysis. The left lobe was fixed with neutral-buffered formalin and submitted for histopathology evaluation to Nationwide Histology for analysis by a certified pathologist. Serum collected at necropsy was used to measure cytokines, such as TNF-α and IL-10. Following intratracheal instillation of LPS at 10 mg/kg, all animals survived for 24 hours post-challenge. We have compiled measurements of key cytokines and chemoattractants believed to be involved in the pathology of acute pulmonary infection and data from the histopathology analysis in the following examples.

### Example 14: The Bioflavonoid Composition Showed a Dose-Correlated, Statistically Significant Reduction in Serum TNF-α

The presence of TNF-α in undiluted rat serum was measured using the rat TNF-α Quantikine ELISA kit from RandD Systems (product #: RTA00) as follows: undiluted serum was added to a microplate coated with TNF-α antibody. After 2 hours at room temperature, TNF-α in serum was bound to the plate and the plate was thoroughly washed. Enzyme-conjugated TNF-α antibody was added to the plate and allowed to bind for 2 hours at room temperature. The washing was repeated, and enzyme substrate was added to the plate. After developing for 30 minutes at room temperature, a stop solution was added, and the absorbance was read at 450 nm. The concentration of TNF-α was calculated based on the absorbance readings of a TNF-α standard curve.

As seen in Table 15, a statistically significant surge in serum TNF-α was observed for vehicle-treated rats challenged intratracheally with LPS. This increase was significantly reduced when rats were treated with UP446, a standardized bioflavonoid composition illustrated in Example 4 and Table 6. Statistically significant and dose-correlated reductions were observed for rats treated with UP446 at 250 mg/kg and 125 mg/kg orally. These reductions in serum TNF-α level was calculated against the vehicle control and found to be 90.7% and 69.8% reductions for the UP446-treated groups at 250 mg/kg and 125 mg/kg, respectively. The positive control, sodium butyrate (SB), showed a statistically significant (67.9%) reduction in serum TNF-α level.

TABLE 15
Effect of the composition on serum TNF-α level.

| Group | Dose (mg/kg) | N | Mean ± SD (pg/mL) | p-value |
|---|---|---|---|---|
| Normal control | 0 | 7 | −1.27 ± 0.93 | 0.000001 |
| Vehicle control | 0 | 10 | 10.43 ± 2.48 | — |
| Sodium Butyrate | 500 | 10 | 3.35 ± 1.73 | 0.000001 |
| UP446 High dose | 250 | 10 | 0.97 ± 1.06 | 0.000001 |
| UP446 Low dose | 125 | 10 | 3.15 ± 0.86 | 0.000001 |

### Example 15: A Standardized Bioflavonoid Composition Showed a Dose-Correlated, Statistically Significant Reduction in Serum IL-1β

The presence of IL-10 in undiluted rat serum was measured using the Rat IL-10 Quantikine ELISA kit from RandD Systems (product #: RLB00) as follows: undiluted serum was added to a microplate coated with IL-1β antibody. After 2 hours at room temperature, IL-1β in serum was bound to the plate and the plate was thoroughly washed. Enzyme-conjugated IL-1β antibody was added to the plate and allowed to bind for 2 hours at room temperature. The washing was repeated, and enzyme substrate was added to the plate. After developing for 30 minutes at room temperature, a stop solution was added, and the absorbance was read at 450 nm. The concentration of IL-1β was calculated based on the absorbance readings of an IL-1β standard curve.

Here again, a dose-correlated and statistically significant reduction of IL-10 was observed for rats treated with UP446, a standardized bioflavonoid composition illustrated in Example 4 and Table 6. A statistically significant increase in the serum level of IL-1β was observed for LPS-induced acute lung injury rats treated with vehicle. Rats treated with UP446 showed 81.2% and 61.8% reductions in the IL-10 level when administered at oral dosages of 250 mg/kg and 125 mg/kg, respectively (Table 16). The sodium butyrate (SB) group showed a 65.3% reduction in serum IL-1β level. These reductions were statistically significant for both the UP446 and Sodium Butyrate (SB) groups.

TABLE 16
Effect of the composition on serum IL-1β level.

| Group | Dose (mg/kg) | N | Mean ± SD (pg/mL) | p-value |
|---|---|---|---|---|
| Normal control | 0 | 7 | −0.14 ± 4.20 | 0.000001 |
| Vehicle control | 0 | 10 | 65.09 ± 13.24 | — |
| Sodium Butyrate | 500 | 10 | 22.58 ± 9.46 | 0.000001 |
| UP446 High dose | 250 | 10 | 12.23 ± 3.55 | 0.000001 |
| UP446 Low dose | 125 | 10 | 24.85 ± 10.10 | 0.000001 |

### Example 16: A Standardized Bioflavonoid Composition Showed a Dose-Correlated and Statistically Significant Reduction IL-6 Level in Broncho-Alveolar Lavage (BAL)

The presence of IL-6 in undiluted rat broncho-alveolar lavage (BAL) was measured using the Rat IL-6 Quantikine ELISA kit from RandD Systems (product #: R6000B) as follows: undiluted BAL was added to a microplate coated with IL-6 antibody. After 2 hours at room temperature, IL-6 in the BAL was bound to the plate and the plate was thoroughly washed. Enzyme-conjugated IL-6 antibody was added to the plate and allowed to bind for 2 hours at room temperature. The washing was repeated, and enzyme substrate was added to the plate. After developing for 30 minutes at room temperature, a stop solution was added, and the absorbance was read at 450 nm. The concentration of IL-6 was calculated based on the absorbance readings of an IL-6 standard curve.

In agreement with the TNF-α and IL-1β data above, UP446, a standardized bioflavonoid composition illustrated in Example 4 and Table 6, showed a dose-correlated and statistically significant reduction in the level of BAL IL-6. While the high dose (250 mg/kg) of UP446 resulted in a 74.6% reduction in the level of BAL IL-6, the lower dose of the bioflavonoid composition showed a 58.3% reduction in the level of BAL IL-6 (Table 17). The reduction was statistically significant for both UP446 at the high and the low dosages when compared to the vehicle-treated acute lung injury rats. The sodium butyrate (SB) group showed a statistically non-significant 37.7% reduction of BAL IL-6 relative to the vehicle-treated disease model

TABLE 17
Effect of the composition on BAL IL-6 level.

| Group | Dose (mg/kg) | N | Mean ± SD (pg/mL) | p-value |
|---|---|---|---|---|
| Normal control | 0 | 7 | 66.41 ± 4.86 | 0.000001 |
| Vehicle control | 0 | 10 | 3103.95 ± 3057.13 | — |
| Sodium Butyrate | 500 | 10 | 1933.30 ± 1744.23 | 0.27 |
| UP446 high dose | 250 | 10 | 787.65 ± 751.17 | 0.002 |
| UP446 low dose | 125 | 10 | 1293.29 ± 794.09 | 0.043 |

### Example 17: A Standardized Bioflavonoid Composition Treatment Produced a Statistically Significant Reduction in CINC-3

CINC-3/macrophage inflammatory protein 2 (MIP-2) belongs to the family of chemotactic cytokines known as chemokines. MIP-2 belongs to the CXC chemokine family, is named CXCL2 and acts through binding of CXCR1 and CXCR2. It is produced mainly by macrophages, monocytes and epithelial cells and is responsible for chemotaxis to the source of inflammation and activation of neutrophils 50 μL of each rat lung homogenate sample (10 per group for vehicle, sodium butyrate (SB), UP446 Low dose, UP446 High dose, 7 per group for control) and 50 μL of assay diluent buffer was added to the wells of a 96-well microplate coated with monoclonal CINC-3 antibody and allowed to bind for 2 hours. The plate was subjected to 5 washes before an enzyme-linked polyclonal CINC-3 was added and allowed to bind for 2 hours. The wells were washed another 5 times before a substrate solution was added to the wells and the enzymatic reaction was allowed to commence for 30 minutes at room temperature protected from light. The enzymatic reaction produced a blue dye that changed to yellow with the addition of the stop solution. The absorbance of each well was read at 450 nm (with a 580 nm correction) and compared to a standard curve of CINC-3 in order to approximate the amount of CINC-3 in each rat lung homogenate sample.

The daily oral treatment of UP446 at 250 mg/kg for a week caused a statistically significant reduction in cytokine-induced neutrophil chemoattractant-3 (CINC-3) in LPS-induced acute lung injury (Table 18). The level of CINC-3 in the normal control rats receiving only the PBS intratracheally was near zero. In contrast, intratracheal LPS-induced acute lung injury rats treated with the carrier vehicle showed an average lung homogenate level of CINC-3 at 563.7±172.9 pg/mL. This level was reduced to an average value of 360.8±110.7 pg/mL for the 250 mg/kg UP446 treated rats. This 36% reduction in CINC-3 level for the rats treated with 250 mg/kg of UP446 was statistically significant when compared to the vehicle-treated disease model. The lower dose UP446 and the sodium butyrate (SB) groups resulted in only marginal 10.5% and 17.7% reductions in lung homogenate CINC-3 level, respectively, in comparison to the vehicle-treated rats.

TABLE 18
Effect of the composition on lung homogenate
MIP-2/CINC-3 activity level.

| Group | Dose (mg/kg) | N | Mean ± SD (pg/mL) | p-value |
|---|---|---|---|---|
| Normal control | 0 | 7 | −4.21 ± 2.38 | 0.0000 |
| Vehicle control | 0 | 10 | 563.71 ± 194.81 | — |
| Sodium Butyrate | 500 | 10 | 464.00 ± 220.32 | 0.2980 |
| UP446 high dose | 250 | 10 | 360.78 ± 150.74 | 0.002 |
| UP446 low dose | 125 | 10 | 504.46 ± 155.20 | 0.1028 |

### Example 18: A Standardized Bioflavonoid Composition Reduced the Total Protein in Broncho-Alveolar Lavage (BAL)

The amount of total protein in in broncho-alveolar lavage (BAL) was measured using the Pierce BCA Protein Assay kit from ThermoFisher Scientific (product #: 23225) as follows: BAL was diluted 1:5, mixed with bicinchoninic acid (BCA) reagent in a microplate, and incubated at 37° C. for 30 minutes. Absorbance was read at 580 nm, and protein concentration in BAL was calculated based on the absorbance readings of a bovine serum albumin standard curve.

A 3-fold increase in the level of total protein from the BAL was found in the LPS-induced acute lung injury rats treated with vehicle compared to the normal control rats. Daily oral treatment of rats for a week with UP446 at 250 mg/kg and 125 mg/kg resulted in 45.1% (p=0.06 vs vehicle) and 36.6% (p=0.21) reductions, respectively, in the content of BAL total proteins when compared to vehicle-treated LPS-induced acute lung injury rats (Table 19). The positive control sodium butyrate (SB) group caused a 30.2% (p=0.27) reduction in the level of BAL total proteins relative to the vehicle-treated LPS-induced acute lung injury rats.

TABLE 19
Effect of the composition on BAL protein level.

| Group | Dose (mg/kg) | N | Mean ± SD (μg/mL) | p-value |
|---|---|---|---|---|
| Normal control | 0 | 7 | 1488.88 ± 322.01 | 0.0037 |
| Vehicle control | 0 | 10 | 4214.86 ± 3311.32 | — |
| Sodium Butyrate | 500 | 10 | 2940.14 ± 2092.32 | 0.2657 |
| UP446 high dose | 250 | 10 | 2314.64 ± 857.27 | 0.0629 |
| UP446 low dose | 125 | 10 | 2673.11 ± 550.77 | 0.2138 |

### Example 19: A Standardized Bioflavonoid Composition Showed a Statistically Significant CRP Reduction in Broncho-Alveolar Lavage (BAL)

The presence of CRP in rat BAL diluted 1:1,000 was measured using the C-Reactive Protein (PTX1) Rat ELISA kit from Abcam (product #: ab108827) as follows: 1:1,000 diluted BAL was added to a microplate coated with CRP antibody. After 2 hours on a plate shaker at room temperature, CRP in BAL was bound to the plate and the plate was thoroughly washed. Biotinylated C Reactive Protein Antibody was added to the plate and allowed to bind for 1 hour on a plate shaker at room temperature. The washing was repeated, and Streptavidin-Peroxidase Conjugate was added to the plate. After incubating for 30 minutes at room temperature, washing was repeated, and chromogen substrate was added. After developing for 10 minutes at room temperature, a stop solution was added, and the absorbance was read at 450 nm. The concentration of CRP was calculated based on the absorbance readings of an CRP standard curve.

A statistically significant 5.6-fold increase in BAL CRP level was observed in the LPS-induced acute lung injury rats treated with vehicle, compared to the normal control rats. Oral treatment of rats for a week with UP446, a standardized bioflavonoid composition illustrated in Example 4 and Table 6, at 250 mg/kg reduced the level of BAL CRP by 42.4% relative to the vehicle-treated disease model (Table 20). This reduction was statistically significant (p<0.05). The positive control sodium butyrate (SB) and the low dose of UP446 group resulted in moderate reduction in CRP level without statistical significance compared to the vehicle-treated diseased rats.

TABLE 20
Effect of the composition on BAL CRP level

| Group | Dose (mg/kg) | N | Mean ± SD (pg/mL) | p-value |
|---|---|---|---|---|
| Normal control | 0 | 7 | 4344.5 ± 3321.6 | 0.0002 |
| Vehicle control | 0 | 10 | 24302.8 ± 8826.1 | — |
| Sodium Butyrate | 500 | 10 | 20093.5 ± 8826.1 | 0.35 |
| UP446 high dose | 250 | 10 | 13987.8 ± 8673.5 | 0.03 |
| UP446 low dose | 125 | 10 | 22223.2 ± 6606.5 | 0.61 |

### Example 20: A Standardized Bioflavonoid Composition Showed a Statistically Significant Reduction of IL-10 in Broncho-Alveolar Lavage (BAL)

The presence of IL-10 in undiluted BAL was measured using the Rat IL-10 Quantikine ELISA kit from RandD Systems (product #: R1000) as follows: undiluted BAL was added to a microplate coated with IL-10 antibody. After 2 hours at room temperature, IL-10 in serum was bound to the plate and the plate was thoroughly washed. Enzyme-conjugated IL-10 antibody was added to the plate and allowed to bind for 2 hours at room temperature. The washing was repeated, and enzyme substrate was added to the plate. After developing for 30 minutes at room temperature, a stop solution was added, and the absorbance was read at 450 nm. The concentration of IL-10 was calculated based on the absorbance readings of an IL-10 standard curve.

The level of the anti-inflammatory cytokine IL-10 was measured in the BAL of diseased rats sacrificed 24 hours post-intratracheal instillation of LPS, following a daily oral treatment of UP446 at 250 mg/kg and 125 mg/kg for 7 days pre-induction. Often, the level of IL-10 corresponds with the severity of infection and inflammatory response needed by the host at the time of infection or injury. As seen in Table 21, the level of IL-10 was found significantly increased 80-fold in in comparison with the normal control rats for the vehicle-treated rats, indicating the high severity of the acute lung injury. In contrast, rats in the UP446 group showed a dose-correlated reduction of IL-10 in the BAL. These reductions were computed and were determined to be 73.6% and 49.2% reductions for UP446 at 250 mg/kg and 125 mg/kg, respectively. The reduction was statistically significant for the high dose (250 mg/kg) of UP446 at p<0.05. At least for this specific model, the reduction in anti-inflammatory cytokine as a result of UP446, a standardized bioflavonoid composition illustrated in Example 4 and Table 6, could be explained by the fact that there could have been a dampening effect in inflammatory response by the host due to mitigation of disease severity and, hence, inflammation by an upstream mechanism, possibly through HMGB1 secretion. Reinforcing this hypothesis, UP446 caused statistically significant reductions in inflammatory cytokines, such as IL-10, IL-6 and TNF-α, leading to a significantly reduced inflammatory response, rendering the need for anti-inflammatory cytokines such as IL-10 less vital to the host. In fact, the level of IL-10 was nearly zero for the normal control group, suggesting induction of anti-inflammatory cytokines is based on the presence or severity of acute lung injury. The significant reduction of IL-10 by the Free-B-Ring flavonoid and flavan composition demonstrated the establishment of the host defense mechanism.

TABLE 21
Effect of the composition on BAL IL-10 level

| Group | Dose (mg/kg) | N | Mean ± SD (pg/mL) | p-value |
|---|---|---|---|---|
| Normal control | 0 | 7 | 2.63 ± 8.35 | 0.004 |
| Vehicle control | 0 | 10 | 207.77 ± 171.33 | — |
| Sodium Butyrate | 500 | 10 | 154.84 ± 159.63 | 0.48 |
| UP446 high dose | 250 | 10 | 54.93 ± 47.70 | 0.02 |
| UP446 low dose | 125 | 10 | 105.55 ± 71.71 | 0.11 |

### Example 21: A Standardized Bioflavonoid Composition Reduced Overall Lung Damage Severity

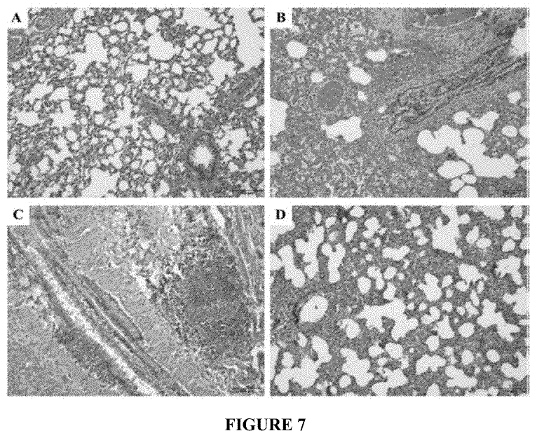

The severity of lung damage as a result of intratracheal LPS was assessed using HandE-stained lung tissue. The left lobe of the lung was used for the histopathology analysis. As seen in Table 22 and FIG. 7, rats in the vehicle-treated group showed statistically significant increases in the severity of lung damage (3.5-fold increase), pulmonary edema (2.5-fold increase) and infiltration of polymorphonuclear (PMN) cells (2.4-fold increase) caused by intratracheal LPS. Daily oral treatment of rats for a week with the high dose of UP446 at 250 mg/kg resulted in a statistically significant 20.8% reduction in overall lung damage severity when compared to vehicle-treated LPS-induced acute lung injury rats. Similarly, a strong trend in the reduction of pulmonary edema (23.3% reduction, p=0.08) was observed for the high dose of UP446 when compared to the vehicle-treated rats. The positive control, sodium butyrate (SB), and the low-dose of the UP446 group caused minimal changes in the histopathology evaluation relative to the vehicle-treated diseased rats.

TABLE 22
Histopathology data from ALI in rats

| | | | Overall Lung | | |
|---|---|---|---|---|---|
| | Dose | | Damage | Pulmonary | Infiltration of |
| Group | (mg/kg) | N | Severity ^{a} | Edema ^{b} | PMN ± cell ^{c} |
| N. Control | 0 | 7 | 0.93 ± 0.49*** | 1.21 ± 0.52*** | 1.14 ± 0.58** |
| Vehicle | 0 | 9 | 3.22 ± 0.58 | 3.00 ± 0.67 | 2.72 ± 0.82 |
| Sodium Butyrate | 500 | 10 | 3.05 ± 0.42 | 2.35 ± 0.95 | 2.75 ± 0.78 |
| UP446 high dose | 250 | 10 | 2.55 ± 0.72* | 2.30 ± 0.84^{d} | 2.55 ± 0.61 |
| UP446 low dose | 125 | 10 | 3.20 ± 0.51 | 2.75 ± 0.78 | 3.20 ± 0.56 |

*P ≤ 0.05;
**P ≤ 0.001;
***P ≤ 0.00001:
^{d}P = 0.08;
SB—Sodium Butyrate;
PMN—polymorphonuclear
^{a} Overall Severity: Norm, mim-mild, mod, severe, ext. severe. Focal, m-focal, regional, reg. ext coalesing, diffuse, Score 0-4.
^{b} Acute Exudative changes: alv, duct and bronch, alv wall and Int edema, congestion, hemorrhage perivasc, alv sac, edema, fibr exud, hemorr alv sac. alv duct thicken dt Hyal membrane type I loss, apoptotic cells, specific parameter scores 0-4
^{c} Inflammatory infiltrative phase: Neutr, other Polymorphs MNC mainly histiocyt and macrophages. BALT alv, interstial, alv-duct, bronchiole diffuse, patch cellular consol, specific parameter scores 0-4

### Example 22: D-Galactose-Induced Immunosenescence Model as an Endogenous and Exogenous Assault Trigger Response

Systemic administration of D-Galactose induces accelerated immune cell senescence, affecting the immune response at the time of challenge, similarly to aged mice. These phenomena are presumed to mimic the immune response profile of the elderly. The novel subject matter UP446, a standardized bioflavonoid composition illustrated in Example 4 and Table 6, was tested in this experimentally-aged mouse model to demonstrate its immune-stimulating effects. Purpose-bred CD-1 mice (12 weeks old) were purchased and used for the accelerated aging study after 2 weeks of acclimation. Mice were randomly assigned to 4 immunized groups and 4 non-immunized groups. The immunized groups included G1=normal control+Vehicle (0.5% CMC), G2=D-Galactose+vehicle, G3=D-Galactose+UP446 200 mg/kg and G4=D-Galactose+UP446 100 mg/kg. The non-immunized treatment groups included G1=normal control+Vehicle (0.5% CMC), G2=D-Galactose+vehicle, G3=D-Galactose+UP446 200 mg/kg and G4=D-Galactose+UP446 100 mg/kg. Ten animals were allocated in each treatment group.

Mice were injected with D-Galactose at 500 mg/kg subcutaneously daily for 10 weeks to induce aging. Four weeks after induction, treatment with 2 doses of UP446 (100 mg/kg-Low dose and 200 mg/kg-High dose) suspended in 0.5% CMC orally commenced for both immunized and non-immunized groups. On the 8^{th} week, each mouse, except those mice in non-immunized groups, was injected with 3 μg of Fluarix quadrivalent IM (2020-2021 influenza season vaccine from GSK. It contained 60 μg hemagglutinin—HA per 0.5 mL single human dose. The vaccine was formulated to contain 15 μg of each of 4 influenza strains such as H1N1, H3N2, B-Victoria lineage and B-Yamagata lineage) for immunization at a single dose.

Daily oral gavaging of UP446 at two doses for the duration of 6 weeks from the 5^{th} week to the 10^{th} week was carried out. At the time of necropsy, (i.e. 14-days after immunization), whole blood (1 mL) was collected and aliquoted—110 μL for flow cytometry immunity panel (delivered on ice to Flow Contract Site Laboratory, Bothell, Wash.), serum was isolated from the remaining blood (about 400 μL serum yield) for antibody ELISAs and enzymatic assays (Unigen, Tacoma Wash.), and 60 μL was shipped in two tubes for cytokine analysis (via Fedex overnight to Sirona DX, Portland, Oreg.). Weights of the thymus and spleen for each animal were measured to determine thymus and spleen indices. Representative images of the thymus and spleen were taken from each group. The spleens were kept on dry ice at the time of necropsy and transferred to −80° C. for future use. Paraformaldehyde and sucrose-fixed thymi were sent to Nationwide histology for Senescence-associated β-galactosidase staining and analysis.

### Example 23: UP446 Produced a Statistically Significant Increase of Thymus Index

Repetitive subcutaneous administration of D-Galactose into mice produces a compromised immune response, resembling changes that occur in the normal aging process. The thymus is one of the most important immune organs ant it would be affected by chronic exposure to D-Gal. The thymus index is a good indication of the strength of the immune function of the body. A higher thymus index corresponds to a normal and stronger non-specific immune response. In the immunized mice, D-Gal mice treated with the vehicle showed a significant reduction (30.3%) in the thymus index compared to the normal control mice. This reduction in thymus index was reversed by both dosages of UP446, a standardized bioflavonoid composition illustrated in Example 4 and Table 6. Mice treated with UP446 orally at 200 mg/kg and 100 mg/kg showed 47.4% and 49.4% increases in thymus index, respectively, when compared to the vehicle-treated D-Gal group. This reversal was statistically significant compared to vehicle-treated D-Gal mice for both doses of UP446. Similarly, the non-immunized mice treated with UP446 at 200 mg/kg and 100 mg/kg also showed a statistically significant increase in the thymus index. These increases were found to be 27.4% and 31.6% when compared to the vehicle-treated D-Gal mice, respectively. It was observed in this study that, regardless of immunization status, UP446 supplementation protected the mice from age-associated thymus involution by injection of D-Galactose.

TABLE 23
In vivo Treatment groups for Thymus protection

| | Thymus Index | | | |
|---|---|---|---|---|
| | Immunized | | Non-immunized | |
| Group | Mean ± Sd | P-value | Mean ± Sd | P-value |
| Normal Control + Vehicle | 0.0020 ± 0.0004 | 0.040 | 0.0023 ± 0.0007 | 0.008 |
| D-Gal. 500 mg/kg + Vehicle | 0.0012 ± 0.0005 | — | 0.0016 ± 0.0003 | — |
| D-Gal + UP446 100 mg/kg | 0.0018 ± 0.0006 | 0.037 | 0.0020 ± 0.0003 | 0.018 |
| D-Gal + UP446 200 mg/kg | 0.0018 ± 0.0004 | 0.018 | 0.0020 ± 0.0002 | 0.004 |

### Example 24: Bioflavonoid Composition Increased Complement C3

Serum was collected at the end of the study and assessed for markers of humoral immunity, such as the C3 component of the complement system. As seen in Table 24, there was a significant decrease in Complement C3 in the immunized normal control group compared to the non-immunized control group. Both immunized D-Gal+UP446 groups had significantly higher Complement C3 than the immunized control group. There was a trend toward an increase in Complement C3 in the non-immunized D-Gal+UP446 treatments compared to the non-immunized D-Gal group, and the immunized D-Gal+200 mg/kg UP446, a standardized bioflavonoid composition illustrated in Example 4 and Table 6, group had a significant increase in Complement C3 compared to the immunized D-Gal group, which demonstrated an enhanced humoral immunity by UP446 for the Immunosenescence animals responding to vaccination.

TABLE 24
Complement C3 in mouse sera from the groups indicated. n = 10 per group.

| Complement C3 | Non- | p value | p value |
|---|---|---|---|
| (μg/mL serum) | immunized | vs Control | vs D-Gal |
| Control | 956 +/− 105 | — | — |
| D-Gal | 805 +/− 146 | 0.201 | — |
| D-Gal + 100 mg/kg UP446 | 909 +/− 72 | 0.565 | 0.330 |
| D-Gal + 200 mg/kg UP446 | 988 +/− 68 | 0.699 | 0.097 |

| | | p value | p value | p value |
|---|---|---|---|---|
| | Immunized | vs Control | vs D-Gal | vs Non-immunized |
| Control | 737 +/− 55 | — | — | *0.012 |
| D-Gal | 798 +/− 52 | 0.224 | — | 0.944 |
| D-Gal + 100 mg/kg UP446 | 868 +/− 79 | *0.046 | 0.255 | 0.548 |
| D-Gal + 200 mg/kg UP446 | 973 +/− 89 | *0.003 | *0.017 | 0.834 |

### Example 25: Effect of the Bioflavonoid Composition on CD3+ T-Cells in Whole Blood (% of Lymphocyte Population)

CD3+CD45+ cells are the T cell population. Expressed as a percentage of all white blood cells (CD45+ cells), we found that the non-immunized animals treated with 200 mg/kg UP446+D-Gal had a trend toward a higher percentage of circulating T cells than the D-Gal group, indicating that UP446, a standardized bioflavonoid composition illustrated in Example 4 and Table 6, increased CD3+ T cell expansion or differentiation in non-immunized animals.

TABLE 25
CD3+ T cells in whole mouse blood

| CD3+ T-cells in whole blood | Non- | p value | p value |
|---|---|---|---|
| (% of lymphocyte population) | immunized | vs Control | vs D-Gal |
| Control | 13.3 +/− 1.55 | — | — |
| D-Gal | 13.0 +/− 1.27 | 0.804 | — |
| D-Gal + 100 mg/kg UP446 | 13.4 +/− 1.64 | 0.978 | 0.788 |
| D-Gal + 200 mg/kg UP446 | 15.8 +/− 1.68 | 0.110 | *0.055 |

| | | p value | p value | p value |
|---|---|---|---|---|
| | Immunized | vs Control | vs D-Gal | vs Non-immunized |
| Control | 12.9 +/− 0.97 | — | — | 0.697 |
| D-Gal | 10.6 +/− 1.31 | *0.037 | — | *0.046 |
| D-Gal +100 mg/kg UP446 | 11.4 +/− 1.32 | 0.160 | 0.499 | 0.147 |
| D-Gal + 200 mg/kg UP446 | 10.1 +/− 1.84 | 0.731 | 0.731 | *0.002 |

### Example 26: Effect of the Bioflavonoid Composition on CD4+ Helper T Cells in Whole Blood (% of Lymphocyte Population)

CD45+CD3+CD4+ cells are Helper T cells, the cells that recognize antigens on antigen-presenting cells and respond with cell division and cytokine secretion. Expressed as a percentage of all white blood cells (CD45+ cells), we found that two weeks after influenza vaccination, the immunized animals treated D-Gal had a significantly lower percentage of circulating Helper T cells than the control group. The immunized D-Gal and D-Gal+UP446 (200 mg/kg) groups also had a significant reduction in CD4+ Helper T cells compared to the non-immunized groups.

TABLE 26
CD3+CD4+ Helper T cells in whole mouse blood

| CD4+ Helper T cells in | | | |
|---|---|---|---|
| whole blood (% of | Non- | p value | p value |
| lymphocyte population) | immunized | vs Control | vs D-Gal |
| Control | 8.46 +/− 0.97 | — | — |
| D-Gal | 8.28 +/− 0.76 | 0.820 | — |
| D-Gal + 100 mg/kg UP446 | 7.98 +/− 1.27 | 0.641 | 0.753 |
| D-Gal + 200 mg/kg UP446 | 9.55 +/− 1.23 | 0.286 | 0.185 |

| | | p value | p value | p value |
|---|---|---|---|---|
| | Immunized | vs Control | vs D-Gal | vs Non-immunized |
| Control | 8.91 +/− 0.71 | — | — | 0.562 |
| D-Gal | 6.72 +/− 0.88 | *0.007 | — | *0.049 |
| D-Gal + 100 mg/kg UP446 | 7.50 +/− 0.91 | 0.070 | 0.343 | 0.633 |
| D-Gal + 200 mg/kg UP446 | 6.40 +/− 1.02 | *0.006 | 0.712 | *0.006 |

### Example 27: Effect of the Bioflavonoid Composition on CD8+ Cytotoxic T Cells in Whole Blood (% of Lymphocyte Population)

CD45+CD3+CD8+ cells are Cytotoxic T cells, the cells that respond to pathogens with cell division and secretion of apoptosis-promoting enzymes to kill infected cells. Expressed as a percentage of all white blood cells (CD45+ cells), the non-immunized animals treated with D-Gal+UP446 (200 mg/kg) had a significant increase in CD8+ Cytotoxic T cells compared to both the non-immunized control and D-Gal groups. The immunized D-Gal+UP446 (200 mg/kg) group had a significantly lower number of Cytotoxic T cells than the non-immunized D-Gal+UP446 (200 mg/kg) group.

TABLE 27
CD3+CD8+ Cytotoxic T cells in whole mouse blood

| CD8+ Cytotoxic T cells | | | |
|---|---|---|---|
| in whole blood (% of | Non- | p value | p value |
| lymphocyte population) | immunized | vs Control | vs D-Gal |
| Control | 4.21 +/− 0.72 | — | — |
| D-Gal | 3.98 +/− 0.61 | 0.703 | — |
| D-Gal + 100 mg/kg UP446 | 4.36 +/− 0.68 | 0.813 | 0.518 |
| D-Gal + 200 mg/kg UP446 | 5.52 +/− 0.64 | *0.045 | *0.013 |

| | | p value | p value | p value |
|---|---|---|---|---|
| | Immunized | vs Control | vs D-Gal | vs Non-immunized |
| Control | 3.24 +/− 0.48 | — | — | 0.094 |
| D-Gal | 3.22 +/− 0.45 | 0.962 | — | 0.130 |
| D-Gal + 100 mg/kg UP446 | 3.30 +/− 0.46 | 0.888 | 0.846 | 0.058 |
| D-Gal + 200 mg/kg UP446 | 3.08 +/− 0.83 | 0.796 | 0.818 | *0.002 |

### Example 28: Effect of the Bioflavonoid Composition on Natural Killer Cells in Whole Blood (% of Lymphocyte Population)

We utilized two different Natural Killer cell markers, mouse CD49b and NKp46, to identify the percentage of Natural Killer cells in the white blood cell population. Natural Killer cells are involved in the innate immune system. When activated, they secrete cytokines and granules to recruit the immune cells and directly cause cell death to cells infected with pathogens, thus they are important for immediate immune responses to pathogens and are active early in systemic infections. CD49b is an integrin that is present specifically on most Natural Killer cells and also a subset of T cells that may be Natural Killer T (NKT) cells. NKp46 is a Natural Cytotoxicity Receptor that is exclusively present on Natural Killer cells and does not mark NKT cells. NKTs and NK-like T cells are also excluded based on their expression of CD3, since NKs are generally CD45+CD3-CD49b+NKp46+(Goh W) (Narni-Mancinelli E). Expressed as a percentage of all white blood cells (CD45+ cells), we found that two weeks after influenza vaccination, the immunized D-Gal group had significantly lower CD3-CD49b+NK cells than the immunized control, or either UP446 treatment (Table 28). This indicated that D-Gal reduced the population of NK cells and hampers the innate immune system's ability to react to pathogens, and that this effect was reversed by UP446, a standardized bioflavonoid composition illustrated in Example 4 and Table 6.

When we looked at the CD3-NKp46+ populations, the non-immunized animals treated with D-Gal+UP446 (100 mg/kg) had a significantly higher percentage of Natural Killer cells than the non-immunized D-gal group, and the immunized D-Gal+UP446 (200 mg/kg) group had a significantly higher percentage of CD3-NKp46+ cells than the immunized D-Gal group (Table 29). The immunized D-Gal+UP446 (200 mg/kg) group also had significantly higher NK cells than the non-immunized D-Gal+UP446 (200 mg/kg) group.

These results indicated that generally, D-Gal+UP446 treatment increased the population of Natural Killer cells compared to the D-Gal treatment alone, in both the non-immunized and immunized animals. This finding indicates that UP446 helps to prime the immune system against pathogens by increasing the population of cells involved in the immediate innate immune response.

TABLE 28
CD3-CD49b+ Natural Killer cells in whole mouse blood

| CD49b+ Natural Killer cells | | | |
|---|---|---|---|
| in whole blood (% of | Non- | p value | p value |
| lymphocyte population) | immunized | vs Control | vs D-Gal |
| Control | 5.12 +/− 0.40 | — | — |
| D-Gal | 4.91 +/− 0.87 | 0.734 | — |
| D-Gal + 100 mg/kg UP446 | 5.52 +/− 0.57 | 0.380 | 0.367 |
| D-Gal + 200 mg/kg UP446 | 5.44 +/− 1.06 | 0.663 | 0.547 |

| | | p value | p value | p value |
|---|---|---|---|---|
| | Immunized | vs Control | vs D-Gal | vs Non-immunized |
| Control | 5.36 +/− 0.81 | — | — | 0.680 |
| D-Gal | 3.76 +/− 0.84 | *0.043 | — | 0.149 |
| D-Gal +100 mg/kg UP446 | 5.35 +/− 0.80 | 0.989 | *0.044 | 0.789 |
| D-Gal + 200 mg/kg UP446 | 5.49 +/− 0.59 | 0.840 | *0.017 | 0.949 |

TABLE 29
CD3-NKp46+ Natural Killer cells in whole mouse blood

| NKp46+ Natural Killer | | | |
|---|---|---|---|
| cells in whole blood | Non- | p value | p value |
| (% of lymphocyte population) | immunized | vs Control | vs D-Gal |
| Control | 4.16 +/− 1.18 | — | — |
| D-Gal | 3.41 +/− 0.67 | 0.397 | — |
| D-Gal + 100 mg/kg UP446 | 4.76 +/− 0.73 | 0.506 | *0.045 |
| D-Gal + 200 mg/kg UP446 | 3.70 +/− 1.06 | 0.653 | 0.719 |

| | | p value | p value | p value vs |
|---|---|---|---|---|
| | Immunized | vs Control | vs D-Gal | Non-immunized |
| Control | 4.85 +/− 1.02 | — | — | 0.494 |
| D-Gal | 4.00 +/− 0.90 | 0.336 | — | 0.415 |
| D-Gal + 100 mg/kg UP446 | 4.88 +/− 0.81 | 0.971 | 0.266 | 0.864 |
| D-Gal + 200 mg/kg UP446 | 5.68 +/− 0.62 | 0.289 | *0.027 | *0.022 |

### Example 29: Effect of the Bioflavonoid Composition on TCRγδ+ Gamma Delta T Cells in Whole Blood (% of Lymphocyte Population)

When we expressed the population of CD4+ Gamma delta T cells as the total number of CD4+ TCRγδ+ cells per μL of blood, there was a significantly higher number of cells in the non-immunized D-Gal+UP446 (200 mg/kg) compared to the non-immunized D-Gal group. The increase in CD4+ TCRγδ+ cells in the D-Gal+UP446 (200 mg/kg) group may have indicated increased immune readiness, or priming.

TABLE 30
CD3+CD4+TCRγδ+ Gamma delta T cells in whole mouse blood

| CD4+TCRγδ+ Gamma | | | |
|---|---|---|---|
| delta T cells in | Non- | p value | p value |
| whole blood (cells/μL) | immunized | vs Control | vs D-Gal |
| Control | 0.94 +/− 0.33 | — | — |
| D-Gal | 0.60 +/− 0.21 | 0.178 | — |
| D-Gal + 100 mg/kg UP446 | 5.21 +/− 6.55 | 0.330 | 0.294 |
| D-Gal + 200 mg/kg UP446 | 1.66 +/− 0.71 | 0.169 | *0.044 |

| | | p value | p value | p value |
|---|---|---|---|---|
| | Immunized | vs Control | vs D-Gal | vs Non-immunized |
| Control | 7.35 +/− 10.4 | — | — | 0.356 |
| D-Gal | 0.91 +/− 0.33 | 0.354 | — | 0.217 |
| D-Gal + 100 mg/kg UP446 | 0.61 +/− 0.21 | 0.332 | 0.237 | 0.296 |
| D-Gal + 200 mg/kg UP446 | 0.92 +/− 0.51 | 0.355 | 0.976 | 0.201 |

### Example 30: Effect of the Bioflavonoid Composition on Serum Cytokines GM-CSF- and Il-12p70

We sent serum isolated from immunized mice two weeks after influenza vaccination for cytokine profiling using Luminex technology. IL-12p70, GM-CSF cytokines had detectable levels of all ten replicates per group. While a reduction in GM-CSF in the D-Gal+UP446 (100 mg/kg) group compared to the D-Gal group approached significance (p=0.058), the reduction in IL-12p70 in the D-Gal+UP446 (200 mg/kg) group compared to the normal control group achieved statistical significance (p=0.010), with no difference between the D-Gal and D-Gal+UP446 (200 mg/kg) groups, perhaps due to variation within the D-Gal group itself.

TABLE 31
Cytokine levels in mouse serum samples

| | IL-12p70 (μg/mL serum) | | | GM-CSF (μg/mL serum) | | |
|---|---|---|---|---|---|---|
| | | P value vs | | | P value vs | |
| Group | Mean +/− SD | control | D-gal | Mean +/− SD | control | D-Gal |
| Control | 109 +/− 4.43 | — | — | 153 +/− 11.7 | — | — |
| D-Gal | 115 +/− 14.6 | 0.577 | — | 170 +/− 14.7 | 0.178 | — |
| D-Gal + | 108 +/− 6.20 | 0.512 | 0.801 | 148 +/− 8.81 | 0.058 | 0.557 |
| 100 mg/kg UP446 | | | | | | |
| D-Gal + | 100 +/− 2.00 | 0.145 | *0.010 | 152 +/− 16.3 | 0.222 | 0.948 |
| 200 mg/kg UP446 | | | | | | |

### Example 31: Effect of the Bioflavonoid Composition on Advanced Glycation End Products (AGEs)

The mechanism by which D-Gal causes an aging phenotype is through the generation of free radicals, especially Advanced Glycation End Products. We sought to measure antioxidation enzyme concentration and free radical levels to determine whether UP446, a standardized bioflavonoid composition illustrated in Example 4 and Table 6, affected this aspect of the mouse model (Azman KF).

We measured Advanced Glycation End Products (AGEs) in the non-immunized and immunized serum samples. We found that the non-immunized D-Gal+UP446 groups had significantly lower AGEs than the non-immunized D-Gal, indicating that UP446 treatment reduced reactive oxygen species under normal physiological conditions.

TABLE 32
Advanced glycation end products of mouse serum

| Advanced Glycation End | | | |
|---|---|---|---|
| Products (mg AGEs/mg | Non- | p value | p value |
| serum protein) | immunized | vs Control | vs D-Gal |
| Control | 30.3 +/− 5.81 | — | — |
| D-Gal | 31.9 +/− 2.47 | 0.707 | — |
| D-Gal + 100 mg/kg UP446 | 21.1 +/− 6.92 | 0.123 | *0.040 |
| D-Gal + 200 mg/kg UP446 | 13.4 +/− 2.97 | *0.001 | *0.0000007 |

| | | p value | p value | p value vs |
|---|---|---|---|---|
| | Immunized | vs Control | vs D-Gal | Non-immunized |
| Control | 18.6 +/− 9.68 | — | — | 0.120 |
| D-Gal | 12.3 +/− 5.62 | 0.390 | — | *0.0003 |
| D-Gal + 100 mg/kg UP446 | 12.6 +/− 3.20 | 0.375 | 0.939 | 0.102 |
| D-Gal + 200 mg/kg UP446 | 10.4 +/− 2.68 | 0.229 | 0.648 | 0.253 |

### Example 32: Effect of the Bioflavonoid Composition on Glutathione Peroxidase

Glutathione peroxidase neutralizes oxygen radicals to prevent oxidative damage to cellular structures, proteins, and nucleic acids. Reactive oxygen species are used as secondary messengers for immune signaling (Ighodaro OM). Increased expression of antioxidation enzymes is indicative of the capability to neutralize excess reactive oxygen species.

We measured the activity of glutathione peroxidase (GSH-Px) in immunized mouse serum samples. We found that there was significantly higher glutathione peroxidase activity in both immunized D-gal+UP446 groups compared to the immunized D-gal group. This indicated an increased capacity to neutralize reactive oxygen species after UP446, a standardized bioflavonoid composition illustrated in Example 4 and Table 6, treatment.

TABLE 33
Glutathione peroxidase content of mouse serum

| Glutathione peroxidase activity | | p value vs | p value vs |
|---|---|---|---|
| (mU/mL serum) | Immunized | Control | D-Gal |
| Control | 114 +/− 5.67 | — | — |
| D-Gal | 114 +/− 6.43 | 0.973 | — |
| D-Gal + 100 mg/kg UP446 | 136 +/− 6.53 | *0.0006 | *0.0001 |
| D-Gal + 200 mg/kg UP446 | 140 +/− 6.41 | *0.0001 | *0.0002 |

### Example 33: Effect of the Bioflavonoid Composition on Protein Expression of NFκB

Statistically significant suppression in the expression of NFκB was observed for mice treated with 200 mg/kg of UP44 in the non-immunized group. NFκB is a transcription factor that is involved in activating immune cells. It is normally inactivated through protein-protein interactions, but during an active host defense response, it is stabilized, translocated to the nucleus, and upregulated. Spleen homogenates were run on SDS-PAGE, transferred, and blotted for the proteins mentioned. Band intensity was measured by densitometry and normalized for each protein of interest to the β-actin loading control. Semi-quantitation of each protein of interest was compared for each group and was found that the non-immunized 200 mg/kg UP446+D-Gal had significantly lower level of NFκB than the D-Gal alone. While for the flu vaccine immunized groups, the bioflavonoid composition UP446+D-Gal group showed statistically significant higher expression of NFκB protein than the normal control group indicating an induced host defense mechanism.

TABLE 34
NFκB protein levels of immunized mouse spleen homogenates
normalized to β-actin and relative to the control group

| NF-κB protein expression | | | |
|---|---|---|---|
| normalized to β-actin and | | | |
| relative to the Non- | Non- | p value | p value |
| immunized Control | immunized | vs Control | vs D-Gal |
| Control | 1.00 +/− 0.26 | — | — |
| D-Gal | 1.51 +/− 0.48 | 0.160 | — |
| D-Gal + 100 mg/kg UP446 | 1.83 +/− 0.52 | *0.043 | 0.497 |
| D-Gal + 200 mg/kg UP446 | 0.64 +/− 0.14 | 0.073 | *0.019 |

| | | p value | p value | p value vs |
|---|---|---|---|---|
| | Immunized | vs Control | vs D-Gal | Non-Immunized |
| Control | 0.69 +/− 0.17 | — | — | 0.838 |
| D-Gal | 1.59 +/− 0.54 | *0.029 | — | 0.153 |
| D-Gal + 100 mg/kg UP446 | 1.67 +/− 0.28 | *<0.001 | 0.844 | 0.107 |
| D-Gal + 200 mg/kg UP446 | 1.97 +/− 0.51 | *0.003 | 0.430 | *<0.001 |

### Example 34: Effect of the Bioflavonoid Composition on Protein Expression of HMGB1

Extracellular HMGB1 is an alarmin protein, involved in escalating the immune response secreted from the nucleus, through the cytoplasm to the circulation. Spleen homogenates were run on SDS-PAGE, transferred, and blotted for the proteins mentioned. Band intensity was measured by densitometry and normalized for each protein of interest to the β-actin loading control. Semi-quantitation of each protein of interest was compared for each group and was found that the non-immunized 200 mg/kg UP446+D-gal and groups had significantly lower level of HMGB1.

TABLE 35
HMGB1 protein levels of immunized mouse spleen homogenates
normalized to β-actin and relative to the control group

| HMGB1 protein expression | | | |
|---|---|---|---|
| normalized to β-actin and | | | |
| relative to the Non- | Non- | p value | p value |
| immunized Control | immunized | vs Control | vs D-Gal |
| Control | 1.00 +/− 0.15 | — | — |
| D-Gal | 0.34 +/− 0.23 | *0.002 | — |
| D-Gal + 100 mg/kg UP446 | 0.12 +/− 0.05 | *<0.001 | 0.156 |
| D-Gal + 200 mg/kg UP446 | 0.03 +/− 0.01 | *<0.001 | 0.053 |

| | | p value | p value | p value |
|---|---|---|---|---|
| | Immunized | vs Control | vs D-Gal | vs Non-immunized |
| Control | 1.40 +/− 0.43 | — | — | 0.263 |
| D-Gal | 1.14 +/− 0.19 | 0.407 | — | *0.001 |
| D-Gal + 100 mg/kg UP446 | 0.98 +/− 0.07 | 0.164 | 0.233 | *<0.001 |
| D-Gal + 200 mg/kg UP446 | 1.45 +/− 0.51 | 0.898 | 0.384 | *0.002 |

### Example 35: The Effects of the Bioflavonoid Composition on Hyperoxia-Induced Mortality in Pseudomonas aeruginosa-Infected Mice

In this study, mice were acclimated for a week before induction. To investigate whether the subject matter disclosed bioflavonoid composition UP446 can reduce animal mortality and increase their survival, mice were exposed to hyperoxia (>90% oxygen for 72 hours) following a treatment with UP446, a standardized bioflavonoid composition illustrated in Example 4 and Table 6, at an oral dose of 250 mg/kg for seven days and treatment was continued for these 3 days before being the mice were inoculated with Pseudomonas Aeruginosa (PA). Mice were observed for 48 hours after bacteria inoculation. Pre-exposure to hyperoxia caused a significantly higher mortality rate (O₂) compared to the mice that remained in room air (RA, Table 36). We found, unexpectedly, substantial mortality 24-hour post PA inoculation in mice exposed to hyperoxia for 48 hours. Compared to the 9% mortality in mice that remained in room air (RA) and received the same amount of PA, 64% mortality was observed in mice treated with vehicle under hyperoxia for 2 days prior to PA inoculation. On the other hand, mice treated prophylactically with resveratrol (RES) and UP446 for 7 days prior to exposure to hyperoxia for 2 days followed by PA inoculation had mortality rates of 27.3%, and 28.6%, respectively, 24 hours post-inoculation. These results suggest that UP446 protected the hosts from oxidative stress and microbial infection that led to reduced mortality. These survival data observed for UP446 are in agreement with the data documented on LPS-induced animal sepsis studies in Examples 10-12, wherein UP446 supplementation produced a statistically significant reduction in mortality.

TABLE 36
The effects of UP446 on hyperoxia-
induced mortality in PA-infected mice

| | | | RES | UP446 |
|---|---|---|---|---|
| | RA | O₂ | (50 mg/kg) | (250 mg/kg) |
| Dead animals | 1 | 9 | 3 | 4 |
| Total animals | 11 | 14 | 11 | 14 |
| Mortality % | 9.09% | 64.29% | 27.27% | 28.57% |

### Example 36: The Effects of the Bioflavonoid Composition on Oxidative Stress-Exacerbated Acute Lung Injury-Induced by Bacterial Infection

To investigate the effects of regulating natural host defense homeostasis, mice were treated with the bioflavonoid composition, UP446, at 250 mg/kg orally for seven days prior to being exposed to >90% oxygen for 48 hours (with continued UP446 treatment) before being inoculated with microbial Pseudomonas aeruginosa (PA). Mice were euthanized 24 hours after bacterial inoculation, the lungs were lavaged, and total protein content was determined from the lung lavage fluid. Pre-exposure to hyperoxia before microbial infection caused a significantly more severe acute lung injury, indicated by the protein edema in these mice (O₂), compared to the mice that remained in room air (RA). The well-known antioxidant—resveratrol (RES), significantly reduced this effect. The reduction in the total protein content in lung lavage fluid of mice in the UP446-treated group was statistically significant compared to that of mice infected with the microbe under hyperoxia and vehicle control (O₂). These results suggest that UP446 can reduce oxidative stress-exacerbated acute lung injury induced by secondary bacterial infection.

TABLE 37
Effect of UP446 on total protein from BAL

| | Dosage | | BAL Total protein content | P-values |
|---|---|---|---|---|
| Group | (mg/kg) | N | (μg/mL) (Mean ± SE) | vs O₂ |
| RA | 0 | 5 | 1297.2 ± 335.0 | 0.0056 |
| O₂ | 0 | 5 | 4616.4 ± 794.9 | — |
| RES | 50 | 3 | 526.0 ± 15.5 | 0.0034 |
| UP446 | 250 | 5 | 1934.2 ± 650.4 | 0.0229 |

Statistical analysis: Dunnett's multiple comparisons test

### Example 37: The Effects of the Bioflavonoid Composition on Bacterial Clearance in the Lung Tissues

Patel et al., 2013 have previously shown that exposure to hyperoxia can compromise host defense against bacterial infections, resulting in higher bacterial loads in lung tissues upon microbial infection. The results in Table 38 indicated that bacterial load was indeed elevated by preexposure to hyperoxia (O₂), compared to that of mice that remained in room air (RA). Corresponding to the significantly reduced lung injury in mice treated with resveratrol and UP446, a standardized bioflavonoid composition illustrated in Example 4 and Table 6, the bacterial load was also significantly reduced in these mice. Data indicated that the differences of the bacterial loads in lung tissues were statistically significant compared to that of microbial-infected mice treated with hyperoxia and vehicle control (O₂). These results suggest that UP446 can regulate natural host defense homeostasis that leads to reduced bacterial load in lung tissues.

TABLE 38
Effect of UP446 on bacterial clearance on lung homogenate

| | Dosage | | ×10⁵ CFU/mL | P-values |
|---|---|---|---|---|
| Group | (mg/kg) | N | (Mean ± SD) | vs O₂ |
| RA | 0 | 8 | 0.63 ± 1.27 | <0.0001 |
| O₂ | 0 | 7 | 27.87 ± 16.19 | — |
| RES | 50 | 5 | 0.02 ± 0.02 | <0.0001 |
| UP446 | 250 | 9 | 3.13 ± 3.44 | <0.0001 |

Statistical analysis: Dunnett's multiple comparisons test

### Example 38: The Effects of the Bioflavonoid Composition on Bacterial Clearance in the Airways

In the above examples, we have shown that exposure to hyperoxia can compromise host defense against bacterial infections, resulting in higher bacterial loads in the lung homogenates Results in Table 39 indicated that bacterial loads in the airways were elevated significantly by preexposure of the mice to hyperoxia (O₂), compared to that of mice that remained in room air (RA). Corresponding to the significantly reduced lung injury in mice treated with resveratrol (RES), the airway bacterial loads were also significantly lower. Similarly, mice treated with UP446 had a significantly lower bacterial load in their airway compared to bacterially infected mice exposed to hyperoxia and treated with vehicle alone. These differences of the bacterial load in the airway was statistically significant compared to that of mice treated with hyperoxia and vehicle control (O₂). These results suggest that UP446, a standardized bioflavonoid composition illustrated in Example 4 and Table 6, can regulate natural host defense homeostasis that leads to reduced bacterial load in airways.

TABLE 39
Effect of UP446 on bacterial clearance in the airways

| | Dosage | | ×10⁵ CFU/mL | P-values |
|---|---|---|---|---|
| Group | (mg/kg) | N | (Mean ± SD) | vs O₂ |
| RA | 0 | 8 | 71.7 ± 62.9 | 0.0255 |
| O₂ | 0 | 7 | 2592.7 ± 1220.3 | — |
| RES | 50 | 5 | 2.4 ± 0.6 | 0.0452 |
| UP446 | 250 | 9 | 303.0 ± 172.1 | 0.0358 |

Statistical analysis: Dunnett's multiple comparisons test

### Example 39: The Effects of the Bioflavonoid Composition on the Accumulation of Extracellular HMGB1 in the Airways

Accumulation of extracellular HMGB1 in the airways can compromise innate immunity, leading to an impaired ability to clear invading pathogens and apoptotic neutrophils. This can subsequently cause acute respiratory tract infections, lung injury and even death (Entezari et al., 2012; Patel et al., 2013). To determine whether UP446-attenuated acute lung injury in bacterially infected mice exposed to hyperoxia is due to its impact on the accumulation of extracellular HMGB1 in the airways, the levels of HMGB1 were measured in the lung lavage fluids. As shown previously, prolonged exposure of these mice to hyperoxia followed by microbial infection increased the accumulation of HMGB1 in the airways. There was a 4.8-fold increase in the level of HMGB1 when mice were exposed to hyperoxia and microbial infection. This elevation can be reduced by pretreatment with either resveratrol (RES) or UP446. Pretreating animals with RES and UP446 showed 74.9% and 71.6% reductions in the level of HMGB1 expression, respectively, compared to vehicle-treated mice exposed to hyperoxia and bacterial infection. These data suggest that the disclosed bioflavonoid composition, UP446, can reduce the accumulation of airway HMGB1 in mice exposed to hyperoxia and bacterial infection. This correlates with the significant enhanced ability of UP446 to improve host defense mechanisms against microbial infection in the respiratory system.

TABLE 40
The effect of UP446 on HMGB1 expression in airways

| | Dosage | | HMGB1 expression | P-values |
|---|---|---|---|---|
| Group | (mg/kg) | N | (AU) (Mean ± SD) | vs O₂ |
| RA | 0 | 5 | 24.8 ± 14.1 | 0.00556 |
| O₂ | 0 | 4 | 116.2 ± 14.6 | — |
| RES | 50 | 7 | 29.2 ± 16.5 | 0.01066 |
| UP446 | 250 | 6 | 33.0 ± 17.6 | 0.01630 |

AU: densitometry arbitrary unit

### Example 40: Effect of the Bioflavonoid Composition on Lung Tissue HMGB1 in SARS-CoV-2 Infected hACE2 Transgenic Mice

The disease model was induced by infecting hACE2 transgenic mice with SARS-CoV-2 virus at 10⁵ TCID₅₀/50 μL via intranasal spray (Bao et al. 2020). Within two hours of SARS-CoV-2 virus nasal spray, mice were administered orally with a bioflavonoid composition, UP894-II illustrated in Example 4 and Table 6, at 400 and 200 mg/kg. Treatment was maintained for a total of 5 daily dosages (i.e. 0 dpi-4 dpi). Normal transgenic control mice without the virus and the disease model (infected with the virus) received only the vehicle (0.5% CMC) at 10 mL/kg volume. Necropsy was performed on 5 dpi. The entire right lung was homogenized for monitoring tissue HMGB1 protein expression.

Lung tissues were excised, snap frozen in liquid nitrogen, and stored at −80° C. until homogenization. Tissues were suspended in lysis buffer at a concentration of 50 mg tissue per 1 mL lysis buffer and homogenized. Samples were placed on ice for 30 minutes, vortexing every five minutes. Samples were centrifuged for 30 minutes and the pellets discarded. Protein was quantified with a BCA assay. Briefly, a 0-10 μg standard curve and BCA working solution (50:1 Reagent A:B) were prepared. 20 μL sample volume was mixed with 200 μL BCA working solution in a microplate and incubated for 30 minutes at 37° C. The plate absorbance was read at 562 nm and the amount of protein was calculated based on the absorbance of the standard curve. 40 μg of protein for each sample were mixed with sodium dodecyl sulfate loading buffer and boiled for 5 minutes at 95-100° C. to yield denatured and reduced protein sample.

Polyacrylamide gels were prepared, and the prepared protein samples were loaded and run with Tris-glycine running buffer (25 mM Tris base, 190 mM glycine, 0.1% SDS, pH 8.3). The gel was transferred via a wet transfer method in transfer buffer (25 mM Tris base, 190 mM glycine, 20% Methanol). The membranes were stained with Ponceau Red to visualize proteins and ensure adequate transfer. Briefly, the membranes were washed in Tris-buffered Saline with 0.1% Tween 20 (TBST). Ponceau Red stock solution was diluted 1:10 and added. The membranes were incubated on an agitator for 5 minutes before being washed extensively in water until the bands were well-defined.

The membranes were blocked and incubated with primary antibodies (1:100-1:3000 dilution) in TBST overnight at 4° C. The membranes were washed three times for five minutes per wash to remove unbound primary antibody. They were incubated in secondary antibodies (1:2000) conjugated to horseradish peroxidase (HRP) in TBST for one hour at room temperature with agitation. The immunoblots were analyzed using a ECL Western blot detection kit (GE Healthcare Life Sciences, Piscataway, N.J., USA) for chemiluminescent detection. Quantification of image data was performed using ImageJ (version 1.41, NIH, Baltimore, Md., USA).

As seen in FIG. 8, vehicle-treated transgenic mice infected with SARS-CoV-2 virus showed a 2-fold increase in lung HMGB1 protein expression compared to the normal transgenic control mice without virus infection. This increase in lung HMGB1 level for the vehicle-treated group was statistically significant compared to the normal control without infection. In contrast, when transgenic mice infected with SARS-CoV-2 virus were treated with a bioflavonoid composition, UP894-II, at two dosages, the expressions of HMGB1 protein in lung tissues were found reduced to the level of the normal control transgenic mice without infection. These reductions in the levels of lung HMGB1 expression as a result of bioflavonoid composition treatment at both high and low dosages were statistically significant compared to vehicle-treated transgenic mice infected with SARS-CoV-2. Reduced HMGB1 in lung tissues indicated an improved host defense mechanism by the disclosed bioflavonoid composition, reducing the potential for lethal cytokine storms and related lung and other organ damage after SARS-Cov-2 coronavirus infection.

### Example 41: Evaluation of the Bioflavonoid Composition UP446 in Human Clinical Trial

Protocol: A randomized, triple-blind, placebo-controlled, parallel clinical trial to investigate a product on supporting immune function in healthy adults. The objective of this study was to investigate the efficacy of the investigational product (IP), UP446 comprising, and in some embodiments consisting of, not less than 60% Free-B-Ring flavonoids and not less than 10% flavans produced in Example 4 and Table 5 and 6 on supporting immune function in healthy adults.

In a randomized, triple-blind, placebo-controlled, parallel study the efficacy of the investigational product on supporting immune function in a healthy adult population in the 28 days before and 28 days after flu vaccination was evaluated. The study included males and females between 40 and 80 years of age, inclusive, who had not yet, but were willing, to receive the influenza vaccine, agreed to provide a verbal history of flu vaccination, agreed to maintain current lifestyle habits as much as possible throughout the study depending on their ability to maintain the following: diet, medications, supplements, exercise, and sleep and avoid taking new supplements, healthy, as determined by medical history and laboratory results, as assessed by Qualified Investigator (QI), willing to complete questionnaires and diaries associated with the study and to complete all clinic visits, and provided voluntary, written, informed consent to participate in the study.

FLUCELVAX® QUAD, Drug Identification Number (DIN) 02494248, is a QIV designed for immunization of adults and children above the age of 9 for the prevention of influenza from subtypes A and B.

TABLE 41
Virus strains in the Flu Vaccine

| Strains | Quantity/Dose |
|---|---|
| Haemagglutinin A/Hawaii/70/2019 (H1N1) | 15 μg |
| pdm09-like virus (A/Nebraska/14/2019) | |
| Haemagglutinin A/Hong Kong/45/2019 | 15 μg |
| (H3N2)-like virus (A/Delaware/39/2019) | |
| Haemagglutinin B/Washington/02/2019-like | 15 μg |
| virus (B/Darwin/7/2019) | |
| Haemagglutinin B/Phuket/3073/2013-like | 15 μg |
| virus (B/Singapore/INFTT-16-0610/2016) | |

Excluded were the following subjects: 1. Women who were pregnant, breast feeding, or planning to become pregnant during the study. 2. Participants with a known allergy to the active or inactive ingredients in UP446, placebo, or influenza vaccine. 3. Unvaccinated participants with flu prior to baseline from September 2020 or prior to Day 28 vaccination. 4. Participants self-reporting a diagnosis of COVID-19 prior to baseline or prior to Day 28 vaccination. 5. Participants who received the COVID-19 vaccine. 6. Current use of prescribed immunomodulators (including corticosteroids), such as immunosuppressants or immunostimulants, within 4 weeks of baseline. 7. Current use of dietary supplement or herbal medicines associated with boosting or modulating the immune system, unless willing to washout.

| Study Arm | Number of Participants |
|---|---|
| UP446 250 mg b.i.d. + Flu Vaccine | N = 25 |
| Placebo 0 mg b.i.d. + Flu Vaccine | N = 25 |
| Total | N = 50 |

TABLE 42
Demographic characteristics of study subjects by treatment groups

| | UP446 | Placebo | P Value |
|---|---|---|---|
| Female | 17 | 16 | 0.9428 |
| Male | 8 | 9 | |
| Age, mean(std) | 25 | 25 | 0.2028 |
| Race | 1 | 4 | 0.0951 |
| Eastern European White | | | |
| Western European White | 20 | 18 | |
| Other | 4 | 3 | |
| Ethnicity | 1 | 1 | 1.0000 |
| Hispanic or Latino | | | |
| Not Hispanic or Latino | 24 | 24 | |
| Marital Status | | | 0.8733 |
| Married | 14 | 16 | |
| Divorced | 1 | 2 | |
| Common-law | 2 | 3 | |
| Separated | 4 | 1 | |
| Single | 3 | 3 | |
| Widow/Widower | 1 | 0 | |

The study subjects were expected to participate in the study for up to a maximum of 56 days. Subjects attended the study at Visit 1 (Screening, Day −45 to −4) for informed consent and at Visit 2 (Baseline, Day 0) for confirmation of eligibility and randomization.

The primary and secondary efficacy and safety endpoints for the study were assessed at Visits 2 (Day 0), Visit 3 (Day 28), and Visit 4 (Day 56). Demographic information and medical history were recorded at the screening visit. Study subjects took the bioflavonoid composition UP446 250 mg two times per day in the morning and evening with meals leading up to an influenza vaccination, (at Day 28), then continued taking daily UP446 250 mg b.i.d. for an addition 4 weeks (up to Day 56).

The primary study outcomes were the difference between UP446 and placebo in the changes in immune parameters as assessed by lymphocyte populations (CD3+, CD4+, CD8+, CD45+, TCRγδ+, CD3−CD16+56+) and immunoglobulins (IgG, IgM, and IgA) in blood from baseline at Day 28 and Day 56.

Statistical analysis was carried out and summary statistics including means, medians, standard deviations, minimums, maximums, proportions (if categorical) on demographic characteristics and outcome measures were obtained for the overall sample and by study groups. Analysis of Variance (ANOVA) was used to examine differences in the averages of continuous variables between the two treatment groups (UP446 and placebo) when normality assumption was satisfied, and Kruskal-Wallis test was used when normality assumption was not satisfied. Chi-square and Fisher exact tests (when cells have counts less than 5) as appropriate were used to investigate differences for categorical variables. Repeated measures analysis of variance (Linear Mixed Model) was used to examine differences in the average values of outcomes over time between the treatment groups. Baseline value was included as a covariate in each model. Repeated measures analysis of variance (Linear Mixed Model) was also used to examine differences in the average values of changes of outcomes over time (from baseline at 28 days, at 56 days and from day 28 at day 56) between the two treatment groups, baseline value was included as a covariate in each model. Pairwise statistical significance from LMM (between groups and within group). Bonferroni adjustment was used for the pairwise comparisons. Statistical significance is defined as p-values <0.05. Statistical Analysis System software version 9.4 (SAS Institute Inc., Cary, N.C., USA) was used to perform the analysis.

Statistically significant outcomes from oral administration of a standardized bioflavonoid composition illustrated in Example 4 and Table 6 were observed for primary end points, such as Immunoglobulin A (IgA) in the preliminary clinical data report. As seen in Table 43, at the end of 8 weeks treatment, subjects who received the bioflavonoid composition, UP446, showed a statistically significant increase in the mucosal immunity indicator immunoglobulin A (IgA) from day 28 to day 56 in comparison to those who received the placebo (P=0.0260). Change in IgA before and after vaccination was 0.08755 g/L higher for participants receiving UP446 compared to those receiving Placebo (p=0.0260). Within groups, subjects who were supplemented with UP446 showed IgA statistically and significantly increased an average of 0.05720 g/L from day 0 to day 56 (p=0.0412) and 0.06280 g/L from day 28 to day 56 (p=0.0252). These data clearly show that IgA, the major immunoglobulin of healthy respiratory system and is thought to be the most important immunoglobulin for mucosal defense, is an important activity of the bioflavonoid composition in regulation of host defense mechanism in human.

TABLE 43
The changes of IgA in UP446 vs Placebo

| IgA | | | Difference between | |
|---|---|---|---|---|
| (g/L) | UP446 | Placebo | UP446 and Placebo | P Value |
| Day 0 | 2.2 +/− 1.2 | 2.1 +/− 0.8 | +0.1 | 0.9752 |
| Day 28 | 2.2 +/− 1.2 | 2.2 +/− 0.9 | 0 | 0.995 |
| Day 56 | 2.3 +/− 1.3 | 2.2 +/− 0.9 | +0.1 | 0.9169 |
| Day 0 | +0.05720 g/L | | +0.04075 g/L | |
| to 56 | p = 0.0412 | | p = 0.2974 | |
| Day 28 | +0.06280 g/L | | +0.08755 g/L | |
| to 56 | p = 0.0252 | | P = 0.0260 | |

The secondary outcomes were the differences between UP446 and placebo at Day 28 and 56 for the following: 1. Number of confirmed COVID-19 infections; 2. Number of confirmed flu cases; 3. Impact of COVID-19 on quality of life assessed by the COVID-19 Impact on QoL Questionnaire; 4. Over-the-counter cold and flu medication use. The difference between UP446 and placebo at Day 56 in: 1. Number of hospitalizations due to COVID-19; 2. Number of hospitalizations due to flu.

Additional outcomes were the difference in changes between UP446, a standardized bioflavonoid composition illustrated in Example 4 and Table 6, and placebo from baseline to those measurements at Day 28 and 56 in the followings: 1. Erythrocyte sedimentation rate (ESR) and C-reactive protein (CRP); 2. Hematology parameters: white blood cell (WBC) count with differential (neutrophils, lymphocytes, monocytes, eosinophils, basophils), reticulocyte count, red blood cell (RBC) count, hemoglobin, hematocrit, platelet count, RBC indices (mean corpuscular volume (MCV), mean corpuscular hemoglobin (MCH), mean corpuscular hemoglobin concentration (MCHC), and red cell distribution width (RDW); 3. Complement C3 and C4 proteins; 4. Mean global severity index, as measured by area under the curve (AUC) for the Modified Wisconsin Upper Respiratory Symptom Survey (WURSS)-24 daily symptom scores. 5. Mean symptom severity scores, as measured by AUC for the WURSS-24 daily severity symptom scores; 6. Number of well days (defined as days scored as 0 (not sick) for the question, “How sick do you feel today?”) as assessed by the Modified WURSS-24 Questionnaire; 7. Number of sick days (defined as days scored as any number from 1 through 7 (sick) for the question, “How sick do you feel today?”) as assessed by the Modified WURSS-24 Questionnaire; 8. Frequency of common upper respiratory tract infection (UTRI) symptoms as assessed by the Modified WURSS-24 Questionnaire; 9. Duration of common UTRI symptoms as assessed by the Modified WURSS-24 Questionnaire; 10. Severity of common UTRI symptoms as assessed by the Modified WURSS-24 Questionnaire; 11. Vitality and quality of life as assessed by the Vitality and Quality of Life (QoL) Questionnaire

Blood samples were collected from each subject in the clinical trial and stored for future analysis to analyze the difference in change between a standardized bioflavonoid composition illustrated in Example 4 and Table 6, and placebo from baseline, at Day 28, and 56 in:

1. Cytokines (GM-CSF; IFN-α; IFN-γ; IL-1α; IL-1β; IL-1RA; IL-2; IL-4; IL-5; IL-6; IL-7; IL-9; IL-10; IL-12 p′70; IL-13; IL-15; IL17A; IL-18; IL-21; IL-22; IL-23; IL-27; IL-31; TNF-α; TNF-β/LTA 150)
2. High mobility group box 1 (HMGB1) protein, nuclear factor kappa B (NF-κB), nuclear factor erythroid 2-related factor 2 (Nrf-2)
3. Oxidative stress as assessed by 8-iso-prostaglandin F2a, catalase (CAT), glutathione peroxidase (GSH-Px), superoxide dismutase (SOD), malondialdehyde (MDA) and advanced glycation end-products (AGEs)
4. Hemagglutinin inhibition (HI) titers for specific strains of virus

In addition to the efficacy analysis, safety evaluations will be performed by testing each blood samples for the followings attributes: 1. Clinical chemistry parameters: alanine aminotransferase (ALT), aspartate aminotransferase (AST), alkaline phosphatase (ALP), total bilirubin, creatinine, electrolytes (Na+, K+, Cl−), estimated glomerular filtration rate (eGFR), glucose; 2. Incidence of pre-emergent and post-emergent adverse events; 3. Vital signs (blood pressure (BP) and heart rate (HR).

### REFERENCES

1. Afolayan A J, Meyer J J. The antimicrobial activity of 3,5,7-trihydroxyflavone isolated from the shoots of Helichrysum aureonitens. J Ethnopharmacol. 1997 August; 57(3):177-81.
2. Andersson U, Ottestad W., and Tracey K J. Extracellular HMGB1: a therapeutic target in severe pulmonary inflammation including COVID-19? Molecular Medicine (2020) 26:42.
3. Angelika Wagner and Birgit Weinberger. Vaccines to Prevent Infectious Diseases in the Older

Population: Immunological Challenges and Future Perspectives. Front. Immunol. 11:717.
4. Angus D C, Yang L, Kong L, Kellum J A, Delude R L, Tracey K J, Weissfeld L; GenIMS Investigators. Circulating high-mobility group box 1 (HMGB1) concentrations are elevated in both uncomplicated pneumonia and pneumonia with severe sepsis. Crit Care Med. 2007 April; 35(4): 1061-7
5. Azman K F, Zakaria R. D-Galactose-induced accelerated aging model: an overview. Biogerontology. 2019 December; 20(6):763-782.
6. Bae J S, Kim N Y, Shin Y Y, Kim S Y and Kim Y J. Activity of catechins and their applications. Biomedical Dermatology (2020) 4:8.
7. Linlin Bao et al, The pathogenecity of SARS-CoV-2 in hACE2 transgenic mic. Nature 2020 July; 583(7818):830-833.
8. Bastianetto S, Zheng W H, Quirion R. Neuroprotective abilities of resveratrol and other red wine constituents against nitric oxide-related toxicity in cultured hippocampal neurons. Br J Pharmacol. 2000 October; 131(4): 711-20.
9. Bianchi M E, Manfredi A A. High-mobility group box 1 (HMGB1) protein at the crossroads between innate and adaptive immunity. Immunol Rev. 2007 December; 220:35-46.
10. Bonneville M, O'Brien R L, Born W K. Gammadelta T cell effector functions: a blend of innate programming and acquired plasticity. Nat Rev Immunol. 2010 July; 10(7):467-78
11. Boumendjel A, Bois F, Beney C, Mariotte A M, Conseil G, Di Pietro A. B-ring substituted 5,7-dihydroxyflavonols with high-affinity binding to P-glycoprotein responsible for cell multidrug resistance. Bioorg Med Chem Lett. 2001 Jan. 8; 11(1):75-7.
12. Chen Y C, Shen S C, Chen L G, Lee T J, Yang L L. Wogonin, baicalin, and baicalein inhibition of inducible nitric oxide synthase and cyclooxygenase-2 gene expressions induced by nitric oxide synthase inhibitors and lipopolysaccharide. Biochem Pharmacol. 2001 Jun. 1; 61(11):1417-27.
13. Chen Y C, Shen S C, Chen L G, Lee T J, Yang L L. Wogonin, baicalin, and baicalein inhibition of inducible nitric oxide synthase and cyclooxygenase-2 gene expressions induced by nitric oxide synthase inhibitors and lipopolysaccharide. Biochem Pharmacol. 2001 Jun. 1; 61(11):1417-27.
14. Chi Y S, Cheon B S, Kim H P. Effect of wogonin, a plant flavone from Scutellaria radix, on the suppression of cyclooxygenase-2 and the induction of inducible nitric oxide synthase in lipopolysaccharide-treated RAW 264.7 cells. Biochem Pharmacol. 2001 May 15; 61(10):1195-203.
15. Chuan-Xin Wu, Hang Sun, Qi Liu, Hui Guo, Jian-Ping Gong. LPS Induces HMGB1 Relocation and Release by Activating the NF-κB-CBP Signal Transduction Pathway in the Murine Macrophage-Like Cell Line RAW264.7. J Surg Res. 2012 Jun. 1; 175(1):88-100.
16. Colby S R. Calculating synergistic and antagonistic responses of herbicide combinations. Weeds 1967; 15:20-2.
17. Commenges D, Scotet V, Renaud S, Jacqmin-Gadda H, Barberger-Gateau P, Dartigues J F. Intake of flavonoids and risk of dementia. Eur J Epidemiol. 2000 April; 16(4):357-63.
18. Derek C Angus, Lihong Yang, Lan Kong, John A Kellum, Russell L Delude, Kevin J Tracey, Lisa Weissfeld, GenIMS Investigators. Circulating High-Mobility Group Box 1 (HMGB1) Concentrations Are Elevated in Both Uncomplicated Pneumonia and Pneumonia With Severe Sepsis. Crit Care Med. 2007 April; 35(4):1061-7.
19. Entezari M, Javdan M, Antoine D J, Morrow D M, Sitapara R A, Patel V, Wang M, Sharma L, Gorasiya S, Zur M, Wu W, Li J, Yang H, Ashby C R, Thomas D, Wang H, Mantell L L. Inhibition of extracellular HMGB1 attenuates hyperoxia-induced inflammatory acute lung injury. Redox Biol. 2014 Jan. 20; 2:314-22.
20. Feng T, Zhou L Y, Gai S C, Zhai Y M, Gou N, Wang X C, Zhang X Y, Cui M X, Wang L B, Wang S W. Acacia catechu (L.f) Willd and Scutellaria baicalensis Georgi extracts suppress LPS-induced pro-inflammatory responses through NF-κB, MAPK, and PI3K-Akt signaling pathways in alveolar epithelial type II cells. Phytotherapy Research. 2019; 33:3251-3260.
21. Gao J, Tian Z, Yang X. Breakthrough: Chloroquine phosphate has shown apparent efficacy in treatment of COVID-19 associated pneumonia in clinical studies. Biosci Trends. 2020; 14(1):72-3.
22. Gautret P, Lagier J C, Parola P, Hoang V T, Meddeb L, Mailhe M, et al. Hydroxychloroquine and azithromycin as a treatment of C OVID-19: results of an open-label non-randomized clinical trial. Int J Antimicrob Agents. 2020:105949.
23. Gentile L F, Moldawer L L. HMGB1 as a therapeutic target for sepsis: it's all in the timing! Expert Opin Ther Targets. 2014 March; 18(3):243-5.
24. HazralK, Mandal A K, Dutta S, Mondal D N, Hazra J. Comprehensive Dossier on Ayurvedic Medicinal plant Acacia catechu Willd.: A Review. Journal of Applied Science And Research, 2017, 5 (3):53-87.
25. Heo M Y, Sohn S J, Au W W. Anti-genotoxicity of galangin as a cancer chemopreventive agent candidate. Mutat Res. 2001 May; 488(2):135-50. doi: 10.1016/s1383-5742(01)00054-0.
26. Hong J, Smith T J, Ho C T, August D A, Yang C S. Effects of purified green and black tea polyphenols on cyclooxygenase- and lipoxygenase-dependent metabolism of arachidonic acid in human colon mucosa and colon tumor tissues. Biochem Pharmacol. 2001 Nov. 1; 62(9):1175-83.
27. Imamura Y, Migita T, Uriu Y, Otagiri M, Okawara T. Inhibitory effects of flavonoids on rabbit heart carbonyl reductase. J Biochem. 2000 April; 127(4):653-8.
28. Itoigawa M, Takeya K, Ito C, Furukawa H. Structure-activity relationship of cardiotonic flavonoids in guinea-pig papillary muscle. J Ethnopharmacol. 1999 June; 65(3):267-72.
29. Jantan I, Ahmad W, and Bukhari S N A. Plant-derived immunomodulators: an insight on their preclinical evaluation and clinical trials. Front. Plant Sci. 2015, 6:655.
30. JIANG H H, DUAN J Y, XU K H and ZHANG W B. Resveratrol protects against asthma-induced airway inflammation and remodeling by inhibiting the HMGB1/TLR4/NF-κB pathway. Experimental And Therapeutic Medicine 18: 459-466, 2019.
31. Kalkbrenner F, Wurm G, von Bruchhausen F. In vitro inhibition and stimulation of purified prostaglandin endoperoxide synthase by flavonoids: structure-activity relationship. Pharmacology. 1992; 44(1):1-12.
32. Kalkbrenner F, Wurm G, von Bruchhausen F. In vitro inhibition and stimulation of purified prostaglandin endoperoxide synthase by flavonoids: structure-activity relationship. Pharmacology. 1992; 44(1):1-12.
33. Kaneko T, Baba N. Protective effect of flavonoids on endothelial cells against linoleic acid hydroperoxide-induced toxicity. Biosci Biotechnol Biochem. 1999 February; 63(2):323-8.
34. Kubo I, Kinst-Hori I, Chaudhuri S K, Kubo Y, Sanchez Y, Ogura T. Flavonols from Heterotheca inuloides: tyrosinase inhibitory activity and structural criteria. Bioorg Med Chem. 2000 July; 8(7): 1749-55.
35. Li N, Liu X X, Hong M, Huang X Z, Chen H, Xu J H, Wang C, Zhang Y X, Zhong J X, Nie H, Gong Q. Sodium butyrate alleviates LPS-induced acute lung injury in mice via inhibiting HMGB1 release. Int Immunopharmacol. 2018 March; 56:242-248.
36. Liang Y C, Tsai S H, Tsai D C, Lin-Shiau S Y, Lin J K. Suppression of inducible cyclooxygenase and nitric oxide synthase through activation of peroxisome proliferator-activated receptor-gamma by flavonoids in mouse macrophages. FEBS Lett. 2001 May 4; 496(1):12-8.
37. Liao H F, Ye J, Gao L, Liu Y L. The main bioactive compounds of Scutellaria baicalensis Georgi. for alleviation of inflammatory cytokines: A comprehensive review. Biomedicine and Pharmacotherapy 133 (2021) 110917.
38. Maria Entezari, Mohammad Javdan, Daniel J Antoine, et al. Inhibition of Extracellular HMGB1 Attenuates Hyperoxia-Induced Inflammatory Acute Lung Injury. Redox Biol. 2014 Jan. 20; 2:314-22.
39. Meyer J J, Afolayan A J, Taylor M B, Erasmus D. Antiviral activity of galangin isolated from the aerial parts of Helichrysum aureonitens. J Ethnopharmacol. 1997 April; 56(2):165-9.
40. Minghua Yang, Lizhi Cao, Min Xie, Yan Yu, Rui Kang, Liangchun Yang, Mingyi Zhao, Daolin Tang. Chloroquine inhibits HMGB1 inflammatory signaling and protects mice from lethal sepsis. Biochem Pharmacol. 2013 Aug. 1; 86(3):410-8.
41. Mutoh M, Takahashi M, Fukuda K, Komatsu H, Enya T, Matsushima-Hibiya Y, Mutoh H, Sugimura T, Wakabayashi K. Suppression by flavonoids of cyclooxygenase-2 promoter-dependent transcriptional activity in colon cancer cells: structure-activity relationship. Jpn J Cancer Res. 2000 July; 91(7):686-91
42. Mutoh M, Takahashi M, Fukuda K, Komatsu H, Enya T, Matsushima-Hibiya Y, Mutoh H, Sugimura T, Wakabayashi K. Suppression by flavonoids of cyclooxygenase-2 promoter-dependent transcriptional activity in colon cancer cells: structure-activity relationship. Jpn J Cancer Res. 2000 July; 91(7):686-91.
43. Noreen Y, el-Seedi H, Perera P, Bohlin L. Two new isoflavones from Ceiba pentandra and their effect on cyclooxygenase-catalyzed prostaglandin biosynthesis. J Nat Prod. 1998 January; 61(1):8-12.
44. Noreen Y, Ringbom T, Perera P, Danielson H, Bohlin L. Development of a radiochemical cyclooxygenase-1 and -2 in vitro assay for identification of natural products as inhibitors of prostaglandin biosynthesis. J Nat Prod. 1998 January; 61(1):2-7.
45. Noreen Y, Serrano G, Perera P, Bohlin L. Flavan-3-ols isolated from some medicinal plants inhibiting COX-1 and COX-2 catalysed prostaglandin biosynthesis. Planta Med. 1998 August; 64(6):520-4.
46. Park J W, Choi Y J, Suh S I, Kwon T K. Involvement of ERK and protein tyrosine phosphatase signaling pathways in EGCG-induced cyclooxygenase-2 expression in Raw 264.7 cells. Biochem Biophys Res Commun. 2001 Aug. 31; 286(4):721-5.
47. Patel V, Dial K, Wu J, Gauthier A G, Wu W. Lin M, Espey M G, Thomas D D, Ashby C R, L L. Dietary Antioxidants Significantly Attenuate Hyperoxia-Induced Acute Inflammatory Lung Injury by Enhancing Macrophage Function via Reducing the Accumulation of Airway HMGB1. Int. J. Mot Sci, 2020, 21, 977.
48. Pilette C, Ouadrhiri Y, Godding V, Vaerman J P, Sibille Y. Lung mucosal immunity: immunoglobulin-A revisited. Eur Respir J. 2001 September; 18(3):571-88.
49. Raso G M, Meli R, Di Carlo G, Pacilio M, Di Carlo R. Inhibition of inducible nitric oxide synthase and cyclooxygenase-2 expression by flavonoids in macrophage J774A.1. Life Sci. 2001 Jan. 12; 68(8):921-31.
50. Ribot J C, Lopes N, Silva-Santos B. γδ T cells in tissue physiology and surveillance. Nat Rev Immunol. 2021 April; 21(4):221-232
51. Shen J, Li P, Liu S S, Liu Q, Li Y, Sun Y H. Traditional uses, ten-years research progress on phytochemistry and pharmacology, and clinical studies of the genus Scutellaria. Journal of Ethnopharinacology 265 (2021) 113198,
52. So F V, Guthrie N, Chambers A F, Carroll K K. Inhibition of proliferation of estrogen receptor-positive MCF-7 human breast cancer cells by flavonoids in the presence and absence of excess estrogen. Cancer Lett. 1997 Jan. 30; 112(2):127-33.
53. Song J W, Long J Y, Xie L, Zhang L L, Xie Q X, Chen H J, Deng M, and Li X F. Applications, phytochemistry, pharmacological effects, pharmacokinetics, toxicity of Scutellaria baicalensis Georgi. and its probably potential therapeutic effects on COVID-19: a review. Chin Med (2020) 15:102.
54. Tordera et al. (1994) Z. Naturforsch [C] 49:235-240
55. Wakabayashi I, Yasui K. Wogonin inhibits inducible prostaglandin E(2) production in macrophages. Eur J Pharmacol. 2000 Oct. 20; 406(3):477-81.
56. Wang H, Bloom O, Zhang M, et al. HMGB-1 as a late mediator of endotoxin lethality in mice. Science 1999; 285:248-51.
57. Wang H, Nair M G, Strasburg G M, Booren A M, Gray I, Dewitt D L. Cyclooxygenase active bioflavonoids from Balaton tart cherry and their structure activity relationships. Phytomedicine. 2000 March; 7(1):15-9.
58. Wang H, Yang H, Czura C J, Sama A E, Tracey K J. HMGB1 as a late mediator of lethal systemic inflammation. Am J Respir Crit Care Med. 2001 Nov. 15; 164(10 Pt 1):1768-73.
59. WEN C C, CHEN H M, YANG N S. Developing Phytocompounds from Medicinal Plants as Immunomodulators. Advances in Botanical Research, Vol. 62. 197-272.
60. Wenzel U, Kuntz S, Brendel M D, Daniel H. Dietary flavone is a potent apoptosis inducer in human colon carcinoma cells. Cancer Res. 2000 Jul. 15; 60(14):3823-31.
61. Wyganowska-Swiatkowska M, Nohawica M, Grocholewicz K, and Nowak G. Influence of Herbal Medicines on HMGB1 Release, SARS-CoV-2 Viral Attachment, Acute Respiratory Failure, and Sepsis. A Literature Review. Int. J. Mol. Sci. 2020, 21, 4639.
62. Yang H, Wang H, Tracey K J. HMGB-1 rediscovered as a cytokine. Shock 2001; 15:247-53.
63. Yang J A, Choi J H, Rhee S J. Effects of green tea catechin on phospholipase A2 activity and antithrombus in streptozotocin diabetic rats. J Nutr Sci Vitaminol (Tokyo). 1999 June; 45(3):337-46.
64. Yang, H., Antoine, D. J., Andersson, U., and Tracey, K. J. (2013). The many faces of HMGB1: molecular structure-functional activity in inflammation, apoptosis and chemotaxis. J. Leukoc. Biol. 93, 865-873.
65. You K M, Jong H G, Kim H P. Inhibition of cyclooxygenase/lipoxygenase from human platelets by polyhydroxylated/methoxylated flavonoids isolated from medicinal plants. Arch Pharm Res. 1999 February; 22(1):18-24.
66. D Altavilla, F Squadrito, A Bitto, F Polito, B P Burnett, V Di Stefanol and L Minutoli. Flavocoxid, a dual inhibitor of cyclooxygenase and 5-lipoxygenase, blunts pro-inflammatory phenotype activation in endotoxin-stimulated macrophages. British Journal of Pharmacology (2009), 157, 1410-1418.
67. Alessandra Bitto, Francesco Squadrito, Natasha Irrera, Gabriele Pizzino, Giovanni Pallio, Anna Mecchio, Federica Galfo, and Domenica Altavilla. Flavocoxid, a Nutraceutical Approach to Blunt Inflammatory Conditions. Mediators of Inflammation. Volume 2014, Article ID 790851, 8 pages
68. Fanfan Zhao1,2 and Yanfen Chang3 and Li Gaol and Xuemei Qinl and Guanhua Du1,4 and Xiang Zhangl,5 and Yuzhi Zhoul Protective effects of Scutellaria baicalensis Georgi extract on D-galactose induced aging rats. Metabolic Brain Disease (2018) 33:1401-1412.

1. A bioflavonoid composition for establishment and regulation of homeostasis of host defense mechanism, comprising at least one standardized bioflavonoid extract enriched for at least one free-B-ring flavonoid and at least one standardized bioflavonoid extract enriched for at least one flavan.

2. The composition of claim 1, wherein the at least one standardized bioflavonoid extract enriched for at least one free-B-ring flavonoid and the at least one standardized bioflavonoid extract enriched for at least one flavan in the composition are in a range of 1%-98% by weight of each extract with the optimized weight ratio of 80:20.

3. The composition of claim 1, wherein the at least one standardized bioflavonoid extract enriched for at least one free-B-ring flavonoid is enriched and standardized from roots of Scutellaria baicalensis; and the at least one standardized bioflavonoid extract enriched for at least one flavan is enriched and standardized from heartwoods of Acacia catechu.

4. The composition of claim 1, wherein the at least one standardized bioflavonoid extract enriched for at least one free-B-ring flavonoid comprises 0.5% to 99.5% of one or more free-B-ring flavonoids.

5. The composition of claim 1 wherein the at least one standardized bioflavonoid extract enriched for at least one flavan comprises 0.5% to 99.5% of catechins.

6. The composition of claim 1 wherein the free-B-ring flavonoid comprises at least one of baicalin, baicalein, baicalein glycoside, wogonin, wogonin glucuronide, wogonin glycoside, oroxylin. oroxylin glycoside, oroxylin glucuronide, chrysin, chrysin glycoside, chrysin glucuronide, scutellarin and scutellarin glycoside, norwogonin, norwogonin glycoside, galangin, or a combination thereof.

7. The composition of claim 1, wherein the at least one standardized bioflavonoid extract enriched for at least one flavan comprises at least one of catechin, epicatechin, catechingallate, gallocatechin, epigallocatechin, epigallocatechin gallate, epitheaflavin, epicatechin gallate, gallocatechingallate, theaflavin, theaflavin gallate, or a combination thereof.

8. The composition of claim 1, wherein the at least one standardized bioflavonoid extract enriched for at least one free-B-ring flavonoid is enriched and standardized from a genus of high plants comprising Desmos, Achyrocline, Oroxylum, Buchenavia, Anaphalis, Cotula, Gnaphalium, Helichrysum, Centaurea, Eupatorium, Baccharis, Sapium, Scutellaria, Molsa, Colebrookea, Stachys, Origanum, Ziziphora, Lindera, Actinodaphne, Acacia, Derris, Glycyrrhiza, Millettia, Pongamia, Tephrosia, Artocarpus, Ficus, Pityrogramma, Notholaena, Pinus, Ulmus, Alpinia, or a combination thereof.

9. The composition of claim 1, wherein the at least one standardized bioflavonoid extract enriched for at least one free-B-ring flavonoid are enriched and standardized from a plant species comprising Scutellaria baicalensis, Scutellaria barbata, Scutellaria orthocalyx, Scutellaria lateriflora, Scutellaria galericulata, Scutellaria viscidula, Scutellaria amoena, Scutellaria rehderiana, Scutellaria likiangensis, Scutellaria galericulata, Scutellaria indica, Scutellaria sessilifolia, Scutellaria viscidula, Scutellaria amoena, Scutellaria rehderiana, Scutellaria likiangensis, Scutellaria orientalis, Oroxylum indicum, Passiflora caerulea, Passiflora incarnata, Pleurotus ostreatus, Lactarius deliciosus, Suillus bellinii, chamomile, carrots, mushroom, honey, propolis, passion flowers, Indian trumpet flower, or a combination thereof.

10. The composition of claim 1, wherein the at least one standardized bioflavonoid extract enriched for at least one flavan is enriched from a plant species comprising Acacia catechu (Black catechu), Senegalia catechu, Acacia concinna, Acacia farnesiana, Acacia Senegal, Acacia speciosa, Acacia arabica, Acacia caesia, Acacia pennata, Acacia sinuata. Acacia mearnsii, Acacia picnantha, Acacia dealbata, Acacia auriculiformis, Acacia holoserecia, Acacia mangium, Anacardium occidentale (Cashew nut testa), Uncaria gambir (White catechu), Uncaria rhynchophylla, Camellia sinensis, Camellia assumica, Euterpe oleracea (acai), Caesalpinia decapetala, Delonix regia, Ginkgo biloba, Acer rubrum, Cocos nucifera, Timonium Brasiliense, Acerola bagasse, Vitellaria paradoxa, Vitis vinifera, Lawsonia inermis, Artocarpus heterophyllus, Medicago sativa, Lotus japonicus, Lotus uliginosus, Eisenia bicyclis, Hedysarum sulfurescens, Robinia pseudoacacia; apple, apricot, prune, cherry, grape leaf, strawberry, beans, lemon, tea, black tea, green tea, red tea, barley grain, green algae (Acetabularia ryukyuensis), red algae (Chondrococcus hornemannii), Chocolate (Cocoa), green coffee beans, or a combination thereof.

11. The composition of claim 1, wherein the at least one standardized bioflavonoid extract enriched for at least one free-B-ring flavonoid and the at least one standardized bioflavonoid extract enriched for at least one flavan are extracted and enriched from a plant part comprising leaves, bark, trunk, trunk bark, stem, stem bark, twigs, tubers, root, rhizome, root bark, bark surface, young shoots, seed, nut, nut testa, fruit, fruit body, mushroom, androecium, gynoecium, calyx, stamen, petal, sepal, carpel (pistil), flower, stem cells, cell culture tissues, or any combination thereof.

12. The composition of claim 1, wherein the standardized bioflavonoid extracts in the composition are extracted with any suitable solvent, including supercritical fluid of CO₂, water, acidic water, basic water, acetone, methanol, ethanol, propenol, butanol, alcohol mixed with water, mixed organic solvents, or a combination thereof.

13. The composition of claim 1, wherein the standardized bioflavonoid extracts are synthesized, metabolized, biodegraded, bioconverted, biotransformed, biosynthesized from small carbon units, by transgenic microbial, by P450 enzymes, by glycotransferase enzyme or a combination of enzymes, by microbacteria, or by a combination thereof.

14. The composition of claim 1, wherein the standardized bioflavonoid extracts are enriched individually or in combination by solvent precipitation, neutralization, solvent partition, ultrafiltration, enzyme digestion, column chromatograph with silica gel, XAD, HP20, LH20, C-18, alumina oxide, polyamide, ion exchange, CG161 resins, or a combination thereof.

15. The composition of claim 1, wherein the composition further comprises a pharmaceutically or nutraceutically acceptable active, adjuvant, carrier, diluent, or excipient, wherein the pharmaceutical or nutraceutical formulation comprises from about 0.1 weight percent (wt %) to about 99.9 wt % of active compounds in the at least one standardized bioflavonoid extract.

16. The composition of claim 1, wherein the active, adjuvant, excipient or carrier comprises Cannabis sativa oil or CBD/THC, turmeric extract or curcumin, terminalia extract, willow bark extract, Aloe vera leaf gel powder, Poria coca extract, rosemary extract, rosmarinic acid, Devil's claw root extract, Cayenne Pepper extract or capsaicin, Prickly Ash bark extract, philodendra bark extract, hop extract, Boswellia extract, rose hips extract, green tea extract, Sophora extract, Withania somnifera, Bupleurum falcatum, Radix Bupleuri, Radix Glycyrrhiza, Fructus forsythiae, Panax quinquefolium, Panax ginseng C. A. Meyer, Korea red ginseng, Lentinula edodes (shiitake), Inonotus obliquus (Chaga mushroom), Lentinula edodes, Lycium barbarum, Phellinus linteus (fruit body), Trametes versicolor (fruit body), Cyamopsis tetragonolobus Cyamopsis tetragonolobus (guar gum), Trametes versicolor, Cladosiphon okamuranus Tokida, Undaria pinnatifida, Mentha or Peppermint extract, ginger or black ginger extract, green tea or grape seed polyphenols, Omega-3 or Omega-6 Fatty Acids, Krill oil, gamma-linolenic acid, citrus bioflavonoids, Acerola concentrate, astaxanthin, pycnogenol, vitamin C, vitamin D, vitamin E, vitamin K, vitamin B, vitamin A, L-lysine, calcium, manganese, Zinc, mineral amino acid chelate(s), amino acid(s), boron and boron glycinate, silica, probiotics, Camphor, Menthol, calcium-based salts, silica, histidine, copper gluconate, CMC, beta-cyclodextrin, cellulose, dextrose, saline, water, oil, shark and bovine cartilage, or a combination thereof.

17. The composition of claim 1, wherein the composition is formulated as a tablet, hard capsule, soft gel capsule, powder, or granule, compressed tablet, pill, gummy, chewing gum, sashay, wafer, bar, or liquid form, tincture, aerial spread, semi solid, semi liquid, solution, emulsion, cream, lotion, ointment, gel base or like form.

18. The composition of claim 1, wherein the composition is effective for respiratory diseases and conditions.

19. The composition of claim 1, wherein the composition is administered via oral, topical, suppository, intravenous, intradermic, intragastric, intramuscular, intraperitoneal, or intravenous routes.

20. The composition of claim 1, wherein the composition treats, manages, or promotes regulation of homeostasis of host defense mechanism in a mammal by administering an effective amount of a composition from 0.01 mg/kg to 500 mg/kg body weight of the mammal.

21. The composition of claim 1, wherein the composition maintains immune homeostasis by optimizing or balancing the immune response; improves aging and immune organ senescence compromised immunity; prevents chronic inflammation and inflammation compromised immunity; helps to maintain a healthy immune response to influenza vaccination and COVID-19 vaccination; helps to maintain a healthy immune function against virus infection and bacterial infections; or protects immune system from oxidative stress damage induced by air pollution of a mammal.

22. The composition of claim 1, wherein the composition regulates HMGB1 as endogenous or exogenous response assault triggers and shifts host defense response to restore homeostasis, the HMGB1 is released by immune senescence, or by inflammation, or by oxidative stress compromised immune cells; by virus, or microbial, air pollutant infected immune cells, host respiratory cells, or cardiovascular cells.

23. The composition of claim 1, wherein the composition regulates HMGB1 by inhibiting HMGB1 release or counteract its action as targeting HMGB1 active or passive release by blocking cytoplasm translocation, or by blocking vesicle mediated release; or inhibiting intramolecular disulfide bond formation in the nucleus; targeting HMGB1 directly upon release and neutralize its effect; blocking HMGB1 pattern recognizing receptors such as Toll-like Receptor (TLR)-2/4/7/9 and receptor for advanced glycation end products (RAGE) or inhibiting their signal transductions; changing the physiochemical microenvironment and preventing formation of HMGB1 tetramer and interfere the binding affinity of HMGB1 to TLR and RAGE; preventing cluster formation or self-association of HMGB1.

24. The composition of claim 1, wherein the composition supports healthy inflammatory response; maintains healthy level of cytokines and cytokine responses to infections; mitigates healthy level of Complement C3 and C4 proteins, cytokines and cytokine responses to infections; mitigates, regulates, and maintains TNF-α, IL-1β, IL-6, GM-CSF; IFN-α; IFN-γ; IL-1α; IL-1RA; IL-2; IL-4; IL-5; IL-7; IL-9; IL-10; IL-12 p′70; IL-13; IL-15; IL17A; IL-18; IL-21; IL-22; IL-23; IL-27; IL-31; TNF-β/LTA, CRP, and CINC3.

25. The composition of claim 1, wherein the composition controls oxidative response and alleviates oxidative stress of the respiratory system; augments antioxidant capacity by increasing SOD and NRF2; decreases advanced glycation end products, increasing Glutathione Peroxidase; neutralizes reactive oxygen species, and prevents oxidative stress caused damage of the structural integrity and loss of function of respiratory, lung and immune system.

26. The composition of claim 1, wherein the composition minimizes or prevents age associated chronic disease caused by AGEs and AGE-RAGE interactions, including prevention of diabetes complications and diabetic microvascular complications in case of diabetes; prevention of severity of coronary atherosclerosis and coronary artery disease in case of cardiovascular disease; prevention of renal failure and end-stage renal disease in case of kidney disease; prevention of hypothalamic dysfunction in case of obesity; mitigation of cancer initiation, progression, migration, invasion, and metastasis; prevention of systemic endotoxemia, inflammation and multiorgan injury in case of Gut microbiome-associated diseases; prevention of neuronal death and degeneration in case of Neurodegenerative diseases; prevention of neuronal apoptosis and neurodegeneration in case of Alzheimer's disease; prevention of neurodegeneration in case of Parkinson's disease; prevention of initiation and progression of non-alcoholic fatty liver disease, inflammatory liver injury nonalcoholic steatohepatitis, hepatic fibrosis and cirrhosis in case of liver disease.

27. The composition of claim 1, wherein the composition improves innate immunity; improves adaptive immunity; increases the activity and count of the white blood cells, enhancing Natural Killer (NK) cell function; increases the count of T and B lymphocytes; increases CD3+, CD4+ NKp46+ Natural Killer cells, TCRγδ+ Gamma delta T cells, and CD4+TCRγδ+ Gamma delta T cells and CD8+ cell counts; and protects and promotes macrophage phagocytic activity.

28. The composition of claim 1, wherein the composition supports or promotes normal antibody IgG, IgM, IgA, Hemagglutinin inhibition (HI) titers for specific strains of virus production or the like of a mammal.

29. The composition of claim 1, wherein the composition neutralizes, reduces, prevents recovery infections from virus comprising highly pathogenic avian influenza (H5N1 virus strain A), influenza A (H1N1, H3N2, H5N1), influenza B/Washington/02/2019-like virus; influenza B/Phuket/3073/2013-like virus, Hepatitis virus A, B, C, and D; Coronavirus SARS-CoV, SARS-CoV-2 (COVID-19) MERS-CoV (MERS), Respiratory syncytial virus (RSV), Enterovirus A71 (EV71) parainfluenza, and adenovirus.

30. The composition of claim 1, wherein the composition neutralizes, reduces, prevents recovery infections of respiratory system from microbial infection comprising Streptococcus pneumoniae, Staphylococcus aureus, Haemophilus influenzae, Pseudomonas aeruginosa, Legionella pneumophila, Moraxella catarrhalis Aspergillus, Cryptococcus, Pneumocystis, Histoplasma capsulatum, Blastomyces, Cryptococcus neoformans, Pneumocystis jiroveci, Candida species (spp.) and Streptococcus pyogenes.

31. The composition of claim 1, wherein the composition neutralizes, reduces, prevents recovery of the damage of respiratory system from PM2.5 particles in air, PM10 particles in air, air pollutants, oxidative smog, smoke from tobacco, electronic cigarette, smoke of recreational marihuana.

32. The composition of claim 1, wherein the composition maintains healthy pulmonary microbiota or symbiotic system in respiratory organs; maintains lung cleanse and detox capability; protects lung structure integrity and oxygen exchanging capacity; maintains respiratory passages and enhances oxygen absorption capacity of alveoli; protects normal healthy lung function from virus infection, bacterial infections and air pollution; mitigates oxidative stress caused pulmonary damage; and promotes microcirculation of the lung and protecting normal coagulation function or the like of a mammal.

33. The composition of claim 1, wherein the composition relieves or reduces cold/flu-like symptoms including but not limited to body aches, sore throat, cough, minor throat and bronchial irritation, nasal congestion, sinus congestion, sinus pressure, runny nose, sneezing, loss of smell, loss of taste, muscle sore, headache, fever and chills; helps loosen phlegm (mucus) and thin bronchial secretions to make coughs more productive; reduces severity of bronchial irritation; reduces severity of lung damage or edema or inflammatory cell infiltration caused by virus infection, microbial infection and air pollution; supports bronchial system and comfortable breathing through the cold/flu or pollution seasons; prevents or treats lung fibrosis; reduces duration or severity of common cold/flu; reduces severity or duration of virus and bacterial infection of respiratory system; prevents, or treats or cures respiratory infections caused by virus, microbial, and air pollutants; manages or treats or prevents, or reverses the progression of respiratory infections; and promotes and strengthens and rejuvenates the repair and renewal function of lung and the entire respiratory system or the like of a mammal.
